(19) United States
(12) Patent Application Publication
(10) Pub. No.: US 2026/0218132 A1
(43) Pub. Date: Jul. 30, 2026

(54) METHODS AND MATERIALS FOR GENERATING STEM CELL-DERIVED ENDOCRINE CELL TYPES

(71) Applicants: Mayo Foundation for Medical Education and Research, Rochester, MN (US); President and Fellows of Harvard College, Cambridge, MA (US)

(72) Inventors: Quinn P. Peterson, Rochester, MN (US); Sharath Belame Shivakumar, Rochester, MN (US); Douglas A. Melton, Cambridge, MA (US)

(21) Appl. No.: 19/147,445

(22) PCT Filed: Jan. 11, 2024
(86) PCT No.: PCT/US2024/011148

§ 371 Date: Jul. 11, 2025

Related U.S. Application Data
(60) Provisional application No. 63/438,477, filed on Jan. 11, 2023.

(51) Int. Cl. C12N 5/071

(52) U.S. Cl. CPC C12N 5/0678; C12N 2500/38; C12N 2501/117; C12N 2501/16; C12N 2501/41; C12N 2501/42; C12N 2501/727; C12N 2506/02

(57) ABSTRACT

This document provides methods and materials for differentiating stem cells into endocrine cell types. For example, this document provides methods and materials for using certain compounds to produce pancreatic delta cells.

### CROSS-REFERENCE TO RELATED APPLICATION

This application claims the benefit of U.S. Provisional Patent Application No. 63/438,477, filed on Jan. 11, 2023, which is incorporated by reference herein in its entirety.

### STATEMENT OF GOVERNMENT INTEREST

This invention was made with Government support under Contract No. 1UC4DK104165-01 awarded by the National Institutes of Health. The U.S. Government has certain rights in the invention.

### TECHNICAL FIELD

This document relates to methods and materials involved in differentiating stem cells into endocrine cell types. For example, this document provides methods and materials for using certain compounds to produce pancreatic delta cells.

### BACKGROUND

Stem cells are characterized by the ability of self-renewal and differentiation into a diverse range of cell types. Control of such differentiation remains a challenge.

### SUMMARY

This document provides methods and materials for differentiating stem cells into endocrine cell types. For example, this document provides methods and materials for using certain compounds to produce pancreatic delta cells. For any compound described herein, the compound may include a salt thereof.

In general, one aspect of this document features a method for producing a cell population comprising delta progenitor (DP) cells. The method can include: culturing a first population of pancreatic progenitor (PP) cells in the presence of a fibroblast growth factor receptor inhibitor and a retinoic acid signaling pathway activator. In some cases, said cell population comprises delta progenitor cells produced from said PP cells of said first population.

The PP cells can be PDX1⁺/NKX6.1⁻ cells. The first population of PP cells can include more than about 80%, 85%, 90%, or 95% Pdx1 positive cells. The first population of PP cells can include less than about 10%, 7%, 5%, 4%, 3%, 2%, or 1% Nkx6.1 positive cells. The fibroblast growth factor receptor inhibitor can be PD173074, AZD4547, erdafitinib, roblitinib, pemigatinib, or BGJ398. The retinoic acid signaling pathway activator can be retinoic acid (RA), TTNPB, or DEAB. Other compounds and therapeutic agents are described herein.

The cell population of delta progenitor cells can include a second population comprising a plurality of somatostatin-positive cells or precursors thereof. The cell population of delta progenitor cells can include a third population of a plurality of glucagon-positive cells, where said second population of said cell population is greater than said third population of said cell population.

In another aspect, one aspect of this document features a cell population comprising delta progenitor (DP) cells. The delta progenitor cells of said cell population can be derived from a first population comprising pancreatic progenitor (PP) cells cultured in the presence of a fibroblast growth factor receptor inhibitor and a retinoic acid signaling pathway activator. The delta progenitor cells of said cell population can express somatostatin, glucagon, insulin, or a combination thereof.

In another aspect, one aspect of this document features a cell population comprising delta progenitor (DP) cells. The delta progenitor cells of said cell population can be produced using a method herein. The delta progenitor cells of said cell population can express somatostatin, glucagon, insulin, or a combination thereof.

In some cases, at least 3 percent of said cell population are delta progenitor cells. In some cases, at least 5 percent or 15 percent of said cell population are delta progenitor cells that express somatostatin. In some cases, 5 percent or less of said cell population are delta progenitor cells that express glucagon. In some cases, 15 percent or less of said cell population are delta progenitor cells that express insulin.

The first population can include a population of PDX1⁺/NKX6.1⁻ cells. The first population can include more than about 80%, 85%, 90%, or 95% Pdx1 positive cells. The first population can include less than about 10%, 7%, 5%, 4%, 3%, 2%, or 1% of Nkx6.1 positive cells. The fibroblast growth factor receptor inhibitor can be PD173074, AZD4547, erdafitinib, roblitinib, pemigatinib, or BGJ398. The retinoic acid signaling pathway activator can be retinoic acid (RA). Other compounds and therapeutic agents are described herein.

In another aspect, one aspect of this document features a method for producing a cell population comprising pre-delta (PD) cells. The method can include: (a) culturing a first population of pancreatic progenitor (PP) cells in the presence of a fibroblast growth factor receptor inhibitor and a retinoic acid signaling pathway activator to form a second population of delta progenitor (DP) cells, and (b) culturing said second population of delta progenitor cells in the presence of a transforming growth factor-β (TGF-β) signaling inhibitor to form said cell population of pre-delta cells.

The PP cells can be PDX1⁺/NKX6.1⁻ cells. The first population of PP cells comprises can include more than about 80%, 85%, 90%, or 95% Pdx1 positive cells. The first population of PP cells can include less than about 10%, 7%, 5%, 4%, 3%, 2%, or 1% of Nkx6.1 positive cells. The fibroblast growth factor receptor inhibitor can be PD173074, AZD4547, erdafitinib, roblitinib, pemigatinib, or BGJ398. The retinoic acid signaling pathway activator can be retinoic acid (RA). The TGF-β signaling inhibitor can be Alk5 inhibitor II, SB431542, or RepSox. Other compounds and therapeutic agents are described herein.

The second population of delta progenitor cells can include a third population comprising a plurality of somatostatin-positive cells or precursors thereof. The second population of delta progenitor cells can include a fourth population of a plurality of glucagon-positive cells, a fifth population of a plurality of insulin-positive cells, or a combination of said fourth population and said fifth population. In some cases, said third population is greater than said fourth population.

In some cases, 5 percent or less of said cell population of pre-delta cells are somatostatin-positive cells. In some cases, the cell population of pre-delta cells comprises a population SC-delta cells, SC-alpha cells, SC-beta cells, non-hormonal cells, polyhormonal cells, or a combination thereof. In some cases, the cell population of pre-delta cells comprises a population of said SC-alpha cells in an amount that is greater than a population of said SC-beta cells.

In another aspect, one aspect of this document features a cell population comprising pre-delta (PD) cells. The pre-delta cells of said cell population can be produced using a method herein. The pre-delta cells of said cell population can express somatostatin, glucagon, insulin, or a combination thereof.

In some cases, 5 percent or less of said cell population are pre-delta cells. In some cases, 5 percent or less of said cell population are pre-delta cells that express somatostatin. In some cases, at least 10 percent of said cell population are pre-delta cells that express glucagon. In some cases, at least 5 percent of said cell population are pre-delta cells that express insulin. In some cases, said cell population comprises a population of SC-delta cells, SC-alpha cells, SC-beta cells, non-hormonal cells, polyhormonal cells, or a combination thereof. In some cases, said cell population comprises a population of said SC-alpha cells in an amount that is greater than a population of said SC-beta cells.

In another aspect, one aspect of this document features a method for producing a cell population comprising stem cell-derived delta (SC-delta) cells. The method can include: culturing a first population of pre-delta (PD) cells in the presence of an adenylyl cyclase activator. In some cases, said cell population comprises SC-delta cells produced from said pre-delta cells of said first population. The adenylyl cyclase activator can include forskolin, NKH 477, PACAP 1-27, or PACAP 1-38. Other compounds and therapeutic agents are described herein.

The first population of pre-delta cells can include a second population comprising a plurality of somatostatin (SST)-positive cells. In some cases, at least 2 percent of said first population of pre-delta cells are SST-positive cells. The first population of pre-delta cells can include a third population of a plurality of cells that express Pdx1, CgA, Pax6, Hhex, Ptch1, or a combination thereof. Other markers are described herein. The first population of pre-delta cells can include a fourth population of polyhormonal cells. In some cases, 50 percent or less of said first population of pre-delta cells comprises said fourth population of polyhormonal cells.

The cell population of SC-delta cells can include a fifth population of a plurality of somatostatin (SST)-positive cells. The cell population of SC-delta cells can include a sixth population of a plurality of cells that express Pdx1, PC2, CgA, Pax6, Hhex, Ptch1, or a combination thereof. Other markers are described herein.

In another aspect, one aspect of this document features a cell population comprising stem cell-derived delta (SC-delta) cells. The SC-delta cells of said cell population can be derived from a first population comprising pre-delta (PD) cells cultured in the presence of an adenylyl cyclase activator. The SC-delta cells of said cell population can express somatostatin, glucagon, insulin, or a combination thereof. The adenylyl cyclase activator can be forskolin, NKH 477, PACAP 1-27, or PACAP 1-38. Other compounds and therapeutic agents are described herein.

In some cases, at least 10 percent of said cell population are SC-delta cells. In some cases, at least 10 percent of said cell population are SC-delta cells that express somatostatin but do not express glucagon and insulin. In some cases, 15 percent or less of said cell population are SC-delta cells that express glucagon. In some cases, 30 percent or less of said cell population are SC-delta cells that express insulin.

The cell population of SC-delta cells can include a second population of a plurality of cells that express Pdx1, Pax6, Hhex, Ptch1, CgA, CgB, PC2, or a combination thereof. Other markers are described herein. The first population of pre-delta cells can include a third population comprising a plurality of somatostatin (SST)-positive cells. In some cases, at least 2 percent of said first population of pre-delta cells are SST-positive cells.

The first population of pre-delta cells can include a fourth population of a plurality of cells that express Pdx1, Pax6, Hhex, Ptch1, or a combination thereof. Other markers are described herein. The first population of pre-delta cells can include a fifth population of polyhormonal cells. In some cases, 50 percent or less of said first population of pre-delta cells comprise said fourth population of polyhormonal cells.

In another aspect, one aspect of this document features a method for producing a cell population comprising stem cell-derived delta (SC-delta) cells. The method can include: (a) culturing a first population of delta progenitor (DP) cells in the presence of a protein kinase C activator and a Notch signaling inhibitor to form a second population of pre-delta (PD) cells, and (b) culturing said second population of pre-delta cells in the presence of an adenylyl cyclase activator to form said cell population of SC-delta cells.

The first population of delta progenitor cells can include a third population comprising a plurality of somatostatin-positive cells or precursors thereof. The protein kinase C activator can be indolactam V, PdBu, or TPPB. The Notch signaling inhibitor can be γ-secretase inhibitor XX, LY411575, or Compound E. The adenylyl cyclase activator can be forskolin, NKH 477, PACAP 1-27, or PACAP 1-38. Other compounds and therapeutic agents are described herein.

The second population of pre-delta cells can include a third population comprising a plurality of somatostatin (SST)-positive cells. In some cases, at least 2 percent of said second population of pre-delta cells are SST-positive cells that do not express glucagon and insulin. The second population of pre-delta cells can include a fourth population of a plurality of cells that express Pdx1, Pax6, Hhex, Ptch1, CgA, CgB, PC2, or a combination thereof. Other markers are described herein. The second population of pre-delta cells can include a fifth population of polyhormonal cells. In some cases, 50 percent or less said second population of pre-delta cells comprises said fifth population of polyhormonal cells. The cell population of SC-delta cells can include a sixth population of somatostatin (SST)-positive cells. The cell population of SC-delta cells can include a seventh population of a plurality of cells that express Pax6, Hhex, Ptch1, CgA, CgB, PC2, or a combination thereof. Other markers are described herein.

In another aspect, one aspect of this document features a cell population comprising stem cell-derived delta (SC-delta) cells. The SC-delta cells of said cell population can be produced using a method herein. The SC-delta cells of said cell population can express somatostatin, glucagon, insulin, or a combination thereof.

In some cases, at least five percent of said cell population are SC-delta cells. In some cases, at least five percent of said cell population are SC-delta cells that express somatostatin but do not express glucagon and insulin. In some cases, 25 percent or less of said cell population are SC-delta cells that express glucagon. In some cases, 60 percent or less of said cell population are SC-delta cells that express insulin. The cell population can include a population of a plurality of cells that express Pdx1, Pax6, Hhex, Ptch1, CgA, CgB, PC2, or a combination thereof. Other markers are described herein.

In another aspect, one aspect of this document features a method for producing a cell population comprising stem cell-derived delta (SC-delta) cells from stem cells. The method can include: (a) culturing a first population of stem cells in the presence of a growth factor from a transforming growth factor-β (TGF-β) superfamily and a glycogen synthase kinase 3 (GSK3) inhibitor to form a second population of definitive endoderm (DE) cells, (b) culturing said second population of DE cells in the presence of a first growth factor from a fibroblast growth factor (FGF) family to form a third population of gut tube endoderm (GTE) cells, (c) culturing said third population of GTE cells in the presence of a second growth factor from a fibroblast growth factor (FGF) family, a first protein kinase C (PKC) activator, a bone morphogenic protein (BMP) signaling pathway inhibitor, a sonic hedgehog (SHH) pathway antagonist, and a first retinoic acid (RA) signaling pathway activator to form a fourth population of pancreatic progenitor (PP) cells, (d) culturing said fourth population of PP cells in the presence of a fibroblast growth factor receptor (FGFR) inhibitor and a second RA signaling pathway activator to form a fifth population of delta progenitor (DP) cells, (e) culturing said fifth population of delta progenitor cells in the presence of a TGF-β signaling inhibitor, or a combination of a second PKC activator and a Notch signaling inhibitor, or a combination of a TGF-β signaling inhibitor, a second PKC activator, and a Notch signaling inhibitor to form a sixth population of pre-delta (PD) cells, and (f) culturing said sixth population of pre-delta cells in the presence of an adenylyl cyclase activator to form said cell population of SC-delta cells.

The first population of stem cells can include one or more embryonic stem cells or induced pluripotent stem cells. The second population of DE cells can be characterized by expression of Sox17, FoxA2, or a combination thereof. The third population of GTE cells can be characterized by expression of Hnf3β. The PP cells can be PDX1^{+/}NKX6.1⁻ cells. The fifth population of delta progenitor cells can include a seventh population of somatostatin-positive cells or precursors thereof. The sixth population of pre-delta cells can include an eighth population of somatostatin-positive cells. The cell population of SC-delta cells can include a ninth population of somatostatin-positive cells. The cell population of SC-delta cells can include a tenth population of a plurality of cells that express Pax6, Hhex, Ptch1, CgA, CgB, PC2, or a combination thereof. Other markers are described herein.

The growth factor from said TGF-β superfamily can be activin A or nodal. The GSK3 inhibitor can be CHIR99021 or Wnt3a. The first growth factor from said FGF family can be keratinocyte growth factor (KGF). The second growth factor from said FGF family can be keratinocyte growth factor (KGF). The first PKC activator can be phorbol 12,13-dibutyrate (PdBu), TPPB, or indolactam V. The BMP signaling pathway inhibitor can be LDN193189 or noggin. The SHH pathway antagonist can be Sant1 or cyclopamine. The RA signaling pathway activator can be retinoic acid or TTNPB. The FGFR inhibitor can be PD173074, AZD4547, erdafitinib, roblitinib, pemigatinib, or BGJ398 (infigratinib). The TGF-β signaling inhibitor can be Alk5 inhibitor II, SB431542, or RepSox. The second PKC activator can be indolactam V, PdBu, or TPPB. The Notch signaling inhibitor can be γ-secretase inhibitor XX, LY411575, or Compound E. The adenylyl cyclase activator can be forskolin, NKH 477, PACAP 1-27, or PACAP 1-38. Other compounds and therapeutic agents are described herein.

In another aspect, one aspect of this document features a method for producing a cell population of stem cell-derived delta (SC-delta) cells. The method can include: (a) culturing a first population of pancreatic progenitor (PP) cells in the presence of a fibroblast growth factor receptor (FGFR) inhibitor and a retinoic acid (RA) signaling pathway activator to form a second population of delta progenitor (DP) cells, (b) culturing said second population of delta progenitor cells in the presence of a transforming growth factor-β (TGF-β) signaling inhibitor, or a combination of a protein kinase C (PKC) activator and a Notch signaling inhibitor, or a combination of a TGF-β signaling inhibitor, a PKC activator, and a Notch signaling inhibitor to form a third population of pre-delta (PD) cells, and (c) culturing said third population of pre-delta cells in the presence of an adenylyl cyclase activator to form said cell population of SC-delta cells.

The PP cells can be PDX1*/NKX6.1⁻ cells. The second population of delta progenitor cells can include a fourth population of somatostatin-positive cells or precursors thereof. The third population of pre-delta cells can include a fifth population of somatostatin-positive cells. The cell population of SC-delta cells can include a sixth population of somatostatin-positive cells. The cell population of SC-delta cells can include a seventh population of a plurality of cells that express Pax6, Hhex, Ptch1, CgA, CgB, PC2, or a combination thereof.

The FGFR inhibitor can be PD173074, AZD4547, erdafitinib, roblitinib, pemigatinib, or BGJ398 (infigratinib). The RA signaling pathway activator can be retinoic acid or TTNPB. The TGF-β signaling inhibitor can be Alk5 inhibitor II, SB431542, or RepSox. The PKC activator can be indolactam V, PdBu, or TPPB. The Notch signaling inhibitor can be 7-secretase inhibitor XX, LY411575, or Compound E. The adenylyl cyclase activator can be forskolin, NKH 477, PACAP 1-27, or PACAP 1-38. Other compounds and therapeutic agents are described herein.

In another aspect, one aspect of this document features a cell population comprising stem cell-derived delta (SC-delta) cells. The SC-delta cells of said cell population can be produced using a method herein. The SC-delta cells of said cell population can express somatostatin, glucagon, insulin, or a combination thereof.

In some cases, at least five percent of said cell population are SC-delta cells. In some cases, at least five percent of said cell population are SC-delta cells that express somatostatin but do not express glucagon and insulin. In some cases, 25 percent or less of said cell population are SC-delta cells that express glucagon. In some cases, 60 percent or less of said cell population are SC-delta cells that express insulin.

The cell population of SC-delta cells can include a population of a plurality of cells that express Pdx1, Pax6, Hhex, Ptch1, CgA, CgB, PC2, or a combination thereof. Other markers are described herein.

The term “agent” as used herein refers to any compound or substance including, without limitation, a small molecule, nucleic acid, polypeptide, peptide, drug, ion, etc. An “agent” can be any chemical, entity, or moiety including, without limitation, synthetic and naturally-occurring proteinaceous and non-proteinaceous entities. In some cases, an agent can be nucleic acid, nucleic acid analogues, proteins, antibodies, peptides, aptamers, oligomer of nucleic acids, amino acids, carbohydrates, ribozymes, DNAzymes, glycoproteins, siRNAs, or lipoproteins. In some cases, an agent can be a small molecule having a chemical moiety. Examples of chemical moieties include unsubstituted or substituted alkyl, aromatic, or heterocyclyl moieties such as macrolides, leptomycins, and related natural products or analogues thereof. In some cases, an agent can be known to have a desired activity and/or property, or can be selected from a library of diverse compounds.

The term “endoderm cell” as used herein refers to a cell that is from one of the three primary germ cell layers in a very early embryo (the other two germ cell layers are the mesoderm and ectoderm). The endoderm layer is the innermost of the three layers. An endoderm cell normally differentiates to give rise first to the embryonic gut and then to the linings of the respiratory and digestive tracts (e.g., the intestine), the liver, and the pancreas.

The term “definitive endoderm” as used herein refers to a cell differentiated from an endoderm cell. In some cases, a definitive endoderm cell can be differentiated into a SC-delta cell (e.g., a pancreatic delta cell). In some cases, a definitive endoderm cell can express a Sox17 polypeptide. Other examples of polypeptides that definitive endoderm cells can express include, without limitation, one or more of MIXL2, GATA4, HNF3b, GSC, FGF17, VWF, CALCR, FOXQ 1, CXCR4, Cerberus, OTX2, goosecoid, C-Kit, CD99, CMKOR 1, and CRIP1 polypeptides. Definitive endoderm cells can have the capacity to differentiate into cells including those of the liver, lung, pancreas, thymus, intestine, stomach and thyroid. The expression of a Sox 17 polypeptide and other polypeptide markers of definitive endoderm can be assessed using any appropriate detection techniques such as immunochemistry, e.g., by using an anti-Sox17 antibody or quantitative RT-PCR.

The term “pancreatic endoderm” refers to a cell of endoderm origin that is capable of differentiating into multiple pancreatic lineages, including pancreatic delta cells, but no longer has the capacity to differentiate into non-pancreatic lineages.

The term “primitive gut tube cell” or “gut tube cell” as used herein refers to a cell differentiated from an endoderm cell and which can be differentiated into a SC-delta cell (e.g., a pancreatic delta cell). A primitive gut tube cell can express one or more of the following markers: a HNF1-β polypeptide, a HNF3-β polypeptide, or a HNF4-α polypeptide. Primitive gut tube cells can have the capacity to differentiate into cells including those of the lung, liver, pancreas, stomach, and intestine. The expression of a HNF1-3 polypeptide and other polypeptide markers of primitive gut tube can be assessed using any appropriate method such as immunochemistry, e.g., by using an anti-HNF1-β antibody.

The term “pancreatic progenitor,” “pancreatic endocrine progenitor,” “pancreatic precursor,” or “pancreatic endocrine precursor” can be used interchangeably herein and refer to a stem cell that is capable of becoming a pancreatic hormone expressing cell capable of forming pancreatic endocrine cells, pancreatic exocrine cells, or pancreatic duct cells. These cells can be committed to differentiating towards at least one type of pancreatic cell such as (a) beta cells that produce insulin, (b) alpha cells that produce glucagon, (c) delta cells (or D cells) that produce somatostatin, and/or (d) F cells that produce pancreatic polypeptide. Such cells can express at least one of the following polypeptide markers: a NGN3 polypeptide, a NKX2.2 polypeptide, a NeuroD polypeptide, an ISL-1 polypeptide, a Pax4 polypeptide, a Pax6 polypeptide, or an ARX polypeptide.

The term “Pdx1-positive, Nkx6.1-negative pancreatic progenitor” as used herein refers to a cell that is a pancreatic progenitor (PP) or pancreatic endoderm (PE) cell having the capacity to differentiate into somatostatin-producing cells such as pancreatic delta cells. A Pdx1-positive, Nkx6.1-negative pancreatic progenitor can express a Pdx1 polypeptide and minimally express a Nkx6.1 polypeptide. Other polypeptide markers of Pdx1-positive, Nkx6.1-negative pancreatic progenitors include, without limitation, a Ptf1a polypeptide, a HNF6 polypeptide, or a Nkx2.2 polypeptide. The expression (or lack of expression) of Pdx1 and Nkx6.1 polypeptides can be assessed using any appropriate method such as immunochemistry or quantitative RT-PCR.

A “precursor thereof” as the term relates to a somatostatin-positive endocrine cell refers to any cell that is capable of differentiating into a somatostatin-positive endocrine cell, including for example, a pluripotent stem cell, a definitive endoderm cell, a primitive gut tube cell, a pancreatic progenitor cell, or an endocrine progenitor cell, when cultured under conditions suitable for differentiating the precursor cell into the somatostatin-positive endocrine cell.

The terms “delta progenitor,” “DP cell,” and “stage 4 cell” refer to cells (e.g., pancreatic delta progenitor cells) that are capable of differentiating into a SC-delta cell or a pre-delta cell or a cell expressing somatostatin or a cell capable of secreting somatostatin. A delta progenitor expresses one or more polypeptide markers indicative of a pancreatic delta progenitor cell including, without limitation, a Pdx1 polypeptide, a CgA polypeptide, or an Arx polypeptide, or a combination thereof. In some instances, expression of Nkx6.1 polypeptide may be excluded.

The terms “pre-delta cell,” “PD cell,” and “stage 5 cell” refer to cells that express at least one marker indicative of a delta cell or that are capable of further maturation to a SC-delta cell or a functional delta cell or a cell capable of secreting somatostatin. A pre-delta cell expresses one or more polypeptide markers indicative of a pancreatic delta cell including, without limitation, an SST polypeptide, a Pdx1 polypeptide, a CgA polypeptide, or low expression of a Pax6 polypeptide, or a combination thereof. A subpopulation of pre-delta cells may additionally express one or more of the following, without limitation, an Hhex polypeptide, a Ptch1 polypeptide, a Gcg polypeptide, or an Ins polypeptide, or a combination thereof. In some instances, expression of Arx polypeptide may be excluded.

The terms “stem cell-derived δ cell,” “SC-δ cell,” “SC-delta cell,” “delta cell,” and “functional pancreatic delta cell” refer to cells (e.g., pancreatic delta cells) that express at least one polypeptide marker indicative of a pancreatic delta cell (e.g., a PDX-1 polypeptide, an Sst polypeptide, a PC2 polypeptide, an Hhex polypeptide, a CgA polypeptide, a Ptch1 polypeptide, high expression of Pax6 polypeptide, or a combination thereof), that excludes expression of non-delta polypeptide markers (e.g., an Ins polypeptide, an Arx polypeptide, or a Gcg polypeptide, or a combination thereof), that express somatostatin, and that display a glucose stimulated somatostatin secretion (GSSS) response characteristic of an endogenous mature delta cell. In some cases, a “SC-delta cell” can be a mature pancreatic delta cell. It is to be understood that a SC-delta cell need not be derived (e.g., directly derived) from stem cells, as the methods and materials provided herein can be used to derive SC-delta cells from any appropriate somatostatin-positive endocrine cell or precursor thereof using any cell as a starting point. For example, embryonic stem cells, induced-pluripotent stem cells, progenitor cells, partially reprogrammed somatic cells (e.g., a somatic cell that has been partially reprogrammed to an intermediate state between an induced pluripotent stem cell and a somatic cell from which it was derived), multipotent cells, totipotent cells, transdifferentiated versions of any of the foregoing cells can be used. In some cases, a SC-delta cell can exhibit a response to one or more glucose challenges (e.g., at least one, at least two, at least three, or more than three sequential glucose challenges). In some cases, the response can be a response that resembles the response of endogenous islets (e.g., human islets) to multiple glucose challenges. In some cases, the morphology of a SC-delta cell can resemble the morphology of an endogenous delta cell. In some cases, a SC-delta cell can package somatostatin into secretory granules.

The terms “progenitor cell” or “precursor cell” can be used interchangeably herein and can refer to a cell that has a cellular phenotype that is more primitive (e.g., is at an earlier step along a developmental pathway or progression than is a fully differentiated cell) relative to a cell that it can give rise to by differentiation. Often, progenitor cells can have a significant or very high proliferative potential. Progenitor cells can give rise to multiple distinct differentiated cell types or to a single differentiated cell type, depending on the developmental pathway and on the environment in which the cells develop and differentiate.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, UniProt KB entry numbers, OMIM entry numbers, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1A-1I: Differentiation of hESCs to delta progenitor cells. (A) Schematic representation of three step differentiation to delta progenitor cells with factors added at each step of differentiation are noted below. (B) Flow cytometry showing the expression of PDX1 and NKX6.1 in stage 3 PP cells. (C) Combinatorial screening strategy for stage 3 PP cells. PDX1 expressing PP cells are treated with combinations of small molecules, and selected hit compounds were used to derive somatostatin positive delta progenitor cells followed by treatment with an endocrine inducer, Alk5 inhibitor II (Alk5i II). (D) Flow cytometric analysis of delta progenitor cells treated with Alk5i II showing approximately 8% of resulting cells express somatostatin. (E) Immunohistochemical staining of a delta progenitor cells cluster. Scale bar=20 μm. (F-H) Differentiated delta progenitor cells secrete pancreatic hormones somatostatin, insulin, and glucagon with somatostatin being the major hormone secreted compared to other two. Data represent mean somatostatin, or insulin, or glucagon secreted per 1000 total cells SEM. For somatostatin n=4, insulin n=10, glucagon n=3, biologically independent delta progenitor replicates were used; whereas, for somatostatin n=3, biologically independent human cadaveric islets replicates were used, and for insulin n=5, biologically independent SCβ replicates were used, and for glucagon n=5, biologically independent SCα replicates were used. (I) Schematic representation of differentiation of PP cells into delta progenitor cells, where maximum somatostatin positive cells can be obtained using simultaneous treatment with both retinoic acid and PD173074 for a period of five days. ESC: embryonic stem cell, DE: definitive endoderm, GTE: gut tube endoderm, PP: pancreatic progenitor, DP: delta progenitor, PD: Pre-delta cell, H. Islets: human cadaveric islets, SCβ: stem cell-derived beta cells, SCα: stem cell-derived alpha cells, KGF: keratinocyte growth factor, RA: retinoic acid, LDN: LDN193185.

FIG. 2A-2J: High throughput small molecules screen (HTS) for derivation of pre-delta and SCδ cells. (A) Schematic of small molecule screen conducted to convert delta progenitor cells into pre-delta cells. (B) Screening results showing the percentage of pre-delta cells generated after introduction of each compound (n=3 biologically independent replicates). (C) The dose response of selected hit compounds from FIG. 2B (Control: n=6 biologically independent replicates, drug treated: n=5 biologically independent replicates). (D) Schematic of small molecule screen conducted to convert pre-delta cells into SCδ cells. (E) Screening results showing the percentage of SCδ cells generated after introduction of each compound (n=3 biologically independent replicates). (F) The dose response of selected hit compound from d (n=6 biologically independent replicates). (G) Schematic of six stages of differentiation of ESCs to SCδ cells. Factors added during each stage of differentiation are noted below.

(H) Immunohistochemical staining of human cadaveric islet, pre-delta cells, and SCδ cells showing expression of somatostatin, insulin, and glucagon. Scale bar=20 μm. (I) Quantification of the percentage of SCα cells, SCβ cells, and SCδ cells generated at stage 4, stage 5, and stage 6 of differentiation using flow cytometer (n=6 biologically independent replicates). (J) Quantification of the percentage of SCα cells, SCP cells, and SCδ cells generated in each SC-α, SC-β, and SC-δ protocol upon differentiation using flow cytometer (SC-α protocol: n=10 biologically independent replicates, SC-β protocol: n=6 biologically independent replicates, SC-δ protocol: n=6 biologically independent replicates).

FIG. 3A-3J: Characterization of pre-delta and SCδ cells. (A-B) Flow cytometric analysis of hormone expression in pre-delta and SCδ cells. (C-D) Quantification of somatostatin secretion in pre-delta cells, SCδ cells, and human islets. (E-F) Quantification of insulin secretion from pre-delta cells, SCδ cells, and human islets. (G-H) Quantification of glucagon secretion from pre-delta cells, SCδ cells, and human islets. Data represent mean somatostatin, or insulin, or glucagon secreted per 1000 total cells±SEM. For pre-delta cells [FIG. 3C, 3E, 3G], somatostatin n=3; insulin n=10; and glucagon n=3 biologically independent replicates. For SCδ cells [FIGS. 3D, 3F, and 3H], somatostatin n=3; insulin n=6; and glucagon n=6 biologically independent replicates. (I) Paracrine signaling from SCδ cells to SCP cells. SCβ cells generated in SC-β protocol were treated with SCδ cell's conditioned media (CM). Insulin secretion was measured under low and high glucose conditions (Control n=5; Treatment n=26 biologically independent replicates). (J) Paracrine signaling from SCδ cells to SCα cells. SCα cells generated in SCα protocol were treated with SCδ cell's conditioned media (CM). Glucagon secretion was measured under low and high glucose conditions (Control n=3; Treatment n=17 biologically independent replicates).

FIG. 4A-4F: SCδ differentiation in the absence of Nkx6.1 expression in PP cells. (A) Schematic of differentiation of SCδ cells from early (PP) and late (PP2) pancreatic progenitors following SC-δ and SC-β protocols respectively. (B) Percentage of Pdx1 and Nkx6.1 expressing cells prior to step 5 of the delta protocol in each variation as quantified by flow cytometer (PP: n=5 biologically independent replicates, PP2: n=3 biologically independent replicates). (C-D) Percentage of SCδ cells or non-hormonal cells obtained after differentiation using either PP or PP2 cells respectively (PP group n=6; PP2 group n=3). (E-F) Immunohistochemical staining of PP-derived and PP2-derived delta progenitor pre-delta and SCδ cells. Scale bar=20 μm. PP=Pancreatic progenitor; PP2=Pancreatic progenitor 2.

FIG. 5A-5C: (A-B) Immunohistochemical staining of delta progenitor, pre-delta, and SCδ cells. Scale bar=20 μm. (C) Electron microscopic images of hESCs, delta progenitor, pre-delta, and SCδ cells. Scale bar=1 μm. The inset with blown up secretory vesicles are indicated by white arrows in FIG. 5C.

FIG. 6A-6D: (A) Immunohistochemical staining of delta progenitor, pre-delta, and SCδ cells. Scale bar=20 μm. (B) Expression of pancreatic endocrine specific genes was evaluated by qRT-PCR, normalized to the expression of GAPDH. The expression of genes in delta progenitor, pre-delta, and SCδ cells was presented as relative mRNA expression to human islets (n=3 biologically independent replicates). (C) Western blot analysis showing change in somatostatin protein levels during differentiation. Lane 1—Ladder; Lane 2—delta progenitor cells; Lane 3—pre-delta cells; Lane 4—SCδ cells; Lane 5—hESCs; Lane 6—Somatostatin positive control. (D) Western blot analysis showing change in somatostatin protein levels in SCδ cells. Lane 1—Ladder; Lane 2—SCδ cells differentiated using early (PDX1NKX6.1⁻) pancreatic progenitor cells; Lane 3—SCδ cells differentiated using late (PDX1+NKX6.1⁺) pancreatic progenitor cells. Somatostatin was detected at 17 kDa, and alpha actin was used as housekeeping protein which was detected at 42 kDa.

FIG. 7A-7D: Delta progenitor cells are differentiated from PP cells using different FGF receptor inhibitors (FGFRi) along with retinoic acid. (A-B) Immunohistochemical staining of delta progenitor cells clusters showing that all FGFR inhibitors used can induce somatostatin. While in the absence of FGFR inhibitors, RA alone is not sufficient to induce the expression of somatostatin under these conditions. Scale bar=20 μm and 50 μm, respectively. (C) The percentage of somatostatin positive cells quantified using flow cytometer after treatment with different FGF receptor inhibitors in various concentrations (n=3 biologically independent replicates under each concentration). (D) The percentage of somatostatin positive cells quantified using flow cytometer after treatment with 200 μM of different FGF receptor inhibitors. The differentiation ability of all tested FGFR inhibitors was comparable to our hit compound PD173074 (n=4 biologically independent replicates).

FIG. 8A-8B: (A) Immunohistochemical staining of delta progenitor, pre-delta, and SCδ cell clusters showing positive staining for somatostatin, insulin, and glucagon. Scale bar=50 μm. (B) Representative bright field images of differentiating clusters taken at different stages of differentiation. Scale bar=200 μm.

FIG. 9A-9D: (a-b) Immunohistochemical staining of delta progenitor, pre-delta, and SCδ cell clusters derived from PP2 cells was analyzed for the expression of delta cell specific markers. Scale bar=20 μm and 50 μm, respectively. (C) Expression of somatostatin and Nkx6.1 in delta progenitor, pre-delta, and SCδ cell clusters derived either from PP or PP2 cells. Higher expression of Nkx6.1 was observed when PP2 cells were used for differentiation into SCδ cells. Scale bar=50 μm. (D) Expression of somatostatin, insulin, and glucagon in delta progenitor, pre-delta, and SCδ cell clusters derived either from PP or PP2 cells. Higher expression of somatostatin was observed in SCδ cells when PP cells were used for differentiation into SCδ cells. Scale bar=50 μm.

FIG. 10A-10B: (A) Immunohistochemical staining of delta progenitor, pre-delta, and SCδ cell clusters showing expression of pancreatic endocrine and delta cell markers. Scale bar=50 μm. (B) Immunohistochemical staining of delta progenitor, pre-delta, and SCδ cell clusters showing expression of somatostatin, insulin, and glucagon. Scale bar=50 μm.

FIG. 11A-11D: (A-B) Immunohistochemical staining of delta progenitor, pre-delta, and SCδ cell clusters showing expression of delta cell marker somatostatin and alpha cell marker ARX. Scale bar=20 μm and 50 μm, respectively. (C) Electron microscopic images of hESC-derived delta progenitor, pre-delta, and SCδ cells. hESCs: Scale bar=5 μm, pre-delta and SCδ: Scale bar=10 μm. (D) Size of secretory vesicles quantified using ImageJ software, the size of secretory vesicles in pre-delta cells are significantly higher than those in delta progenitor and SCδ cells.

FIG. 12A-12D: (A) Full blot image showing somatostatin (17 kDa) and alpha actin (42 kDa) protein bands in delta progenitor, pre-delta, and SCδ cells after differentiation from early pancreatic progenitor cells. Protein lysates were prepared from 3 differentiation flasks. (C) Similar to FIG. 12A, except that the differentiation was carried out from late pancreatic progenitor cells. Somatostatin protein was not detected in delta progenitor cells both in FIG. 12A and FIG. 12C, and it gradually increased in pre-delta and SCδ cells in FIG. 12A. However, the level of somatostatin protein remained same in both pre-delta and SCδ cells in FIG. 12C (n=3 biologically independent replicates). (B, D) Graphical representative of the intensity of somatostatin bands normalized to the intensity of alpha actin as shown in FIG. 12A and FIG. 12C, respectively (n=3 biologically independent replicates).

FIG. 13A-13B: (A-B) Quadrant plots showing the percentage of cells expressing PDX1 and NKX6.1 as quantified by flow cytometer in early (n=7 biologically independent replicates) and late (n=3 biologically independent replicates) pancreatic progenitor cells respectively. Early pancreatic progenitors do not express sufficient NKX6.1, as compared to late pancreatic progenitors.

FIG. 14A-14B: (A) Quadrant plots showing the percentage of cells expressing somatostatin, insulin, and glucagon as quantified by flow cytometer in delta progenitor (DP) cells (n=6 biologically independent replicates). (B) The bottom graph is a summary of percentage of expression of hormones. Data are presented as mean±SEM.

FIG. 15A-15B: (A) Quadrant plots showing the percentage of cells expressing somatostatin, insulin, and glucagon as quantified by flow cytometer in pre-delta (PD) cells (n=6 biologically independent replicates). (B) The bottom graph is a summary of percentage of expression of hormones. Data are presented as mean±SEM.

FIG. 16A-16B: (A) Quadrant plots showing the percentage of cells expressing somatostatin, insulin, and glucagon as quantified by flow cytometer in SCδ cells (n=6 biologically independent replicates). (B) The bottom graph is a summary of percentage of expression of hormones. Data are presented as mean±SEM.

FIG. 17A-17B: (A) Quadrant plots showing the percentage of cells expressing somatostatin, insulin, and glucagon as quantified by flow cytometer in long-term cultured SCδ cells (n=4 biologically independent replicates). (B) The bottom graph is a summary of percentage of expression of hormones. Data are presented as mean±SEM.

FIG. 18A-18C: (A) Forward and side scatter plot was used to select events (top) and forward height and width plot (bottom) was used to select singlets in all our analysis. (B) Quadrant plots showing the expression of somatostatin, insulin, and glucagon. (C) Different gating strategies were applied on FIG. 18B to characterize eight different subpopulations of cells based on the expression of somatostatin, insulin, and glucagon.

FIG. 19A-19C: (A) Graph showing the percentage of eight different cell subpopulations present in delta progenitor, pre-delta, and SCδ cells. (B) Similar to FIG. 19A but SCδ cells were differentiated from late pancreatic progenitor cells. (C) Similar to FIG. 19A but the culture was extended for two more weeks confirming the stability of differentiated delta cells and further enrichment of monohormonal SC-δ cells without undergoing dedifferentiation.

FIG. 20A-20B: (A-B) Immunohistochemical staining showing somatostatin expressing cells in either delta progenitor, pre-delta, or SCδ cell clusters are not expressing ki67 and are non-proliferative. Scale bar=20 μm and 50 μm, respectively.

FIG. 21A-21B: TUNEL assay showing somatostatin positive cells in either delta progenitor, pre-delta, or SCδ cell clusters are not positive for TMRred and hence are not undergoing apoptosis. Scale bar=20 μm (in FIG. 21A) and 50 μm (in FIG. 21B).

FIG. 22A-22C: High throughput small molecules screen was conducted at (A) 0.2 μM, (B) 2 μM, and (C) 20 μM concentrations during step 5 of differentiation. The graphs represent the percentage of cells expressing only somatostatin (pre-delta cells). The threshold was set to 3 times the standard deviation of all the compounds. Anything above threshold value was considered as hit (n=3 biologically independent replicates).

FIG. 23A-23C: High throughput small molecules screen was conducted at (A) 0.2 μM, (B) 2 μM, and (C) 20 μM concentrations during step 6 of differentiation. The graphs represent the percentage of cells expressing only somatostatin (SCδ cells). The threshold was set to 3 times the standard deviation. Anything above threshold value was considered as hit (n=3 biologically independent replicates). The cytotoxic hits are omitted for further experimentation and are indicated with red letters.

FIG. 24A-24D: (A) The dose response of selected hit compound from step 5 screen (Control: n=6 biologically independent replicates, drug treated: n=5 biologically independent replicates). (B) The dose response of selected hit compound from step 6 screen (n=6 biologically independent replicates). (C) The percentage of cells expressing only somatostatin is quantified using flow cytometer after step 5 of differentiation. The delta progenitor cells are allowed step 5 differentiation in either basal media S3 without any factors and/or in presence of Alk5inh II, and/or Alk5inh II, Indolactam V, and γ-secretase inhibitor XX, and/or Indolactam V and γ-secretase inhibitor XX. The presence of Alk5inh II, Indolactam V, and γ-secretase inhibitor XX together in S3 resulted in better differentiation with higher cells expressing only somatostatin (basal media: n=4 biologically independent replicates, basal media+Alk5inh II: n=3 biologically independent replicates, basal media+Alk5inh II+Indolactam V+γ-secretase inhibitor XX: n=6 biologically independent replicates, basal media+Indolactam V+γ-secretase inhibitor XX: n=4 biologically independent replicates). (D) Step 6 differentiation was carried out in presence of either forskolin or both forskolin and Indolactam V (n=4 biologically independent replicates).

FIG. 25A-25H: (A, B, E, F) Immunohistochemical staining of pre-delta and SCδ cells showing the expression of somatostatin, insulin, and glucagon before and after high throughput small molecule screen conducted during step 5 (in FIG. 25A-35B) and step 6 (in FIG. 25E-F) of differentiation. An increase in monohormonal pre-delta cells was noted in FIG. 25A compared to FIG. 25B, similarly, an increase in monohormonal SCδ cells was noted in FIG. 25E compared to FIG. 25F. (C) The percentage of polyhormonal cells reduced while monohormonal pre-delta cells increased in presence of Indolactam V and 7-secretase inhibitor XX as quantified by flow cytometer (Step 5 with Indolactam V and γ-secretase inhibitor XX: n=6 biologically independent replicates, Step 6 without Indolactam V, and γ-secretase inhibitor XX: n=3 biologically independent replicates). (D) Quantification and comparison of eight different cell subpopulations present during step 5 of differentiation in presence or absence of Indolactam V and γ-secretase inhibitor XX (Step 5 with Indolactam V and γ-secretase inhibitor XX: n=6 biologically independent replicates, Step 6 without Indolactam V, and γ-secretase inhibitor XX: n=3 biologically independent replicates). (G) The percentage of monohormonal SCδ cells increased in presence of forskolin during step 6 of differentiation (Step 6 with forskolin: n=6 biologically independent replicates, Step 6 without forskolin: n=4 biologically independent replicates). (H) Quantification and comparison of eight different cell subpopulations present during step 6 of differentiation in presence or absence of forskolin (Step 6 with forskolin: n=6 biologically independent replicates, Step 6 without forskolin: n=4 biologically independent replicates).

FIG. 26A-26B: (A-B) Quadrant plots showing the expression of somatostatin, insulin, and glucagon as quantified by flow cytometer in pre-delta and SCδ cells, respectively (PD: n=3 biologically independent replicates, SC&: n=4 biologically independent replicates). The population of cells positive for somatostatin, negative for glucagon in quadrant plots drawn for somatostatin versus glucagon are allowed in the quadrant plots drawn for somatostatin versus insulin to find the percentage of cells expressing only somatostatin. The percentage of cells expressing only somatostatin is given under each plot.

### DETAILED DESCRIPTION

This document provides methods and materials for differentiating stem cells into endocrine cell types. For example, this document provides methods and materials for using certain compounds to produce pancreatic delta cells.

Cell-based therapies have shown great potential to alleviate the growing burden of various diseases, including Type 1 diabetes. Such therapies would benefit from human pancreatic beta cells that properly respond to and secrete insulin upon glucose flux in the body. However, within the islets, endocrine cells experience paracrine and autocrine regulations for proper functioning. The lack of knowledge on such regulations in pancreatic endocrine systems has limited the success of stem cell derived beta cell therapeutics. Despite success in generation of stem cell derived beta and alpha cells, the generation of other endocrine cell types is not yet accomplished. As described herein, a high-throughput screening (HTS) platform was used to identify agents (e.g., small molecules) that convert human embryonic stem cells into pancreatic delta cells. The identification of these agents (e.g., small molecules) can lead to the development of new protocols that can advance cell-based therapeutic regimens. Accordingly, the document provides compositions that include stem cell-derived delta cells as well as methods for generating them and methods for using them.

Any appropriate method can be used to determine whether or not cells formed from stem cells are desirable SC-delta cells (e.g., glucose-responsive, somatostatin (SST)-secreting delta cells). For example, an immunohistochemistry assay can be performed to confirm the formation of SST-secreting delta cells.

Once obtained, the SC-delta cells can be administered to a mammal (e.g., a human) to treat, for example, diabetes (e.g., type 1 diabetes). For example, SC-delta cells (e.g., alone or in combination with SC-beta cells) can be transplanted into a human under a renal capsule, within the liver, within a fat pad, or subcutaneously.

Various cell populations can be assessed or determined to have one or more markers. Examples of markers include one or more of the following: UniProtKB No. P52945, PDX1 HUMAN, Pancreas/duodenum homeobox protein 1 (PDX1 or Pdx1), OMIM Entry No. 600733, UniProtKB No. P78426, NKX61_HUMAN, Homeobox protein Nkx-6.1 (NKX6-1, NKX6.1, or Nkx6.1), OMIM Entry No. 602563; UniProtKB No. P10645, CMGA_HUMAN, Chromogranin A (CHGA, ChgA, or CgA), OMIM Entry No. 118910; UniProtKB No. P05060, SCG1_HUMAN, Secretogranin-1 (SCG1) or Chromogranin B (CHGB, ChgB, or CgB), OMIM Entry No. 118920; UniProtKB No. Q96QS3, ARX_HUMAN, Homeobox protein ARX (ARX or Arx), OMIM Entry No. 300382; UniProtKB No. Q03014, HHEX_HUMAN, Hematopoietically-expressed homeobox protein HHEX (HHEX or Hhex), OMIM Entry No. 604420; UniProtKB No. Q13635, PTC 1_HUMAN, Protein patched homolog 1 (PTCH1 or Ptch1), OMIM Entry No. 601309; UniProtKB No. P61278, SMS_HUMAN, Somatostatin (SST or Sst), OMIM Entry No. 182450; UniProtKB No. P26367, PAX6_HUMAN, Paired box protein Pax-6 (Pax6), OMIM Entry No. 607108; UniProtKB No. P16519, NEC2_HUMAN, Neuroendocrine convertase 2 (PCSK2) or Proprotein convertase (PC2), OMIM Entry No. 162151; UniProtKB No. P01308, INS_HUMAN, Insulin (INS or Ins), OMIM Entry No. 176730; and UniProtKB No. P01275, GLUC_HUMAN, Pro-glucagon (GCG or Gcg), OMIM Entry No. 138030, in which each of these is incorporated herein by reference in its entirety.

### Generation of Precursor Cells, Including Stem Cells, Endoderm-Derived Cells, Pancreatic Progenitor Cells, Delta Progenitor Cells, and Pre-Delta Cells

Any appropriate method can be used to obtain stem cells such as embryonic stem (ES) cells or induced pluripotent stem (iPS) cells. In some cases, any appropriate cell type can be used to obtain iPS cells. For example, skin, lung, heart, liver, blood, kidney, or muscle cells can be used to obtain iPS cells. Such cells can be obtained from any type of mammal including, without limitation, humans, mice, rats, dogs, cats, cows, pigs, or monkeys. In addition, any stage of the mammal can be used such as mammals at the embryo, neonate, newborn, or adult stage. For example, fibroblasts obtained from an adult human patient can be used to obtain iPS cells. Such iPS cells can be used to treat that same human patient (or to treat a different human) or can be used to create differentiated cells that can be used to treat that same human patient (or a different human). For example, somatic cells from a human patient can be treated as described herein to obtain iPS cells. The obtained iPS cells can be differentiated into SC-delta cells as described herein that can be implanted into that same human patient.

Any appropriate method can be used to generate definitive endoderm (DE) cells. In some cases, DE cells can be generated by differentiating stem cells into DE cells. For example, a growth factor from a transforming growth factor-β (TGF-β) superfamily and a glycogen synthase kinase 3 (GSK3) inhibitor can be used to differentiate stem cells into DE cells. Examples of growth factors from the TGF-β superfamily that can be used to differentiate stem cells into DE cells include, without limitation, activin A (e.g., UniProtKB No. P08476, INHBA_HUMAN (inhibin beta A chain)) and nodal (e.g., UniProtKB No. Q96S42, NODAL_HUMAN (nodal homolog)). Examples of GSK3 inhibitors that can be used to differentiate stem cells into DE cells include, without limitation, CHIR99021 (CAS No. 252917-06-9, 6-[2-[[4-(2,4-dichlorophenyl)-5-(5-methyl-1H-imidazol-2-yl)pyrimidin-2-yl]amino]ethylamino]pyridine-3-carbonitrile) and Wnt3a (e.g., UniProtKB No. P56704, WNT3A_HUMAN (protein Wnt-3a)).

In some cases, DE cells can be characterized by expression of Sox17 (e.g., UniProtKB No. Q9H6I2, SOX17 HUMAN (transcription factor SOX-17)), FoxA2 (e.g., UniProtKB No. Q9Y261, FOXA2_HUMAN (hepatocyte nuclear factor 3-beta)), or a combination thereof.

Any appropriate method can be used generate gut tube endoderm (GTE) cells. In some cases, GTE cells can be generated by differentiating DE cells into GTE cells. For example, a growth factor from a fibroblast growth factor (FGF) family can be used to differentiate DE cells into GTE cells. Examples of growth factors from the FGF family that can be used to differentiate DE cells into GTE cells include, without limitation, keratinocyte growth factor (KGF) (e.g., UniProtKB No. P21781, FGF7_HUMAN (fibroblast growth factor 7)).

In some cases, GTE cells can be characterized by expression of Hnf3P (e.g., UniProtKB No. Q9Y261, FOXA2_HUMAN (hepatocyte nuclear factor 3-beta)).

Any appropriate method can be used to generate pancreatic progenitor (PP) cells. In some cases, PP cells can be generated by differentiating GTE cells into PP cells. For example, a growth factor from a fibroblast growth factor (FGF) family, a protein kinase C (PKC) activator, a bone morphogenic protein (BMP) signaling pathway inhibitor, a sonic hedgehog (SHH) pathway antagonist, and/or a retinoic acid (RA) signaling pathway activator can be used to differentiate GTE cells into PP cells. Examples of growth factors from the FGF family that can be used to differentiate GTE cells into PP cells include, without limitation, keratinocyte growth factor (KGF).

Examples of PKC activators include phorbol 12,13-dibutyrate (PdBu) (CAS No. 37558-16-0 or 61557-88-8, Catalog No. 4153 from Tocris Bioscience (Bristol, United Kingdom), (1 aR,1bS,4aR,7aS,7bS,8R,9R,9aS)-1a,1b,4,4a,5,7a,7b,8,9,9a-decahydro-4a,7b-dihydroxy-3-(hydroxymethyl)-1,1,6,8-tetramethyl-5-oxo-1H-cyclopropa[3,4]benz[1,2-e]azulen-9,9a-diyl butanoic acid ester), TPPB (CAS No. 497259-23-1, Catalog No. 5343 from Tocris Bioscience, (2E,4E)-N-[(2S,5S)-1,2,3,4,5,6-hexahydro-5-(hydroxymethyl)-1-methyl-2-(1-methylethyl)-3-oxo-1,4-benzodiazocin-8-yl]-5-[4-(trifluoromethyl)phenyl]-2,4-pentadienamide), and indolactam V (CAS No. 90365-57-4, Catalog No. 14647 from Cayman Chemical (Ann Arbor, MI), (10S,13S)-13-(hydroxymethyl)-9-methyl-10-propan-2-yl-3,9,12-triazatricyclo[6.6.1.04,15]pentadeca-1,4(15),5,7-tetraen-11-one).

Examples of BMP signaling pathway inhibitors that can be used to differentiate GTE cells into PP cells include, without limitation, LDN193189 (CAS No. 1062368-24-4, 4-[6-(4-piperazin-1-ylphenyl)pyrazolo[1,5-a]pyrimidin-3-yl]quinolin) and noggin (e.g., UniProtKB No. Q13253, NOGG_HUMAN (noggin)). Examples of SHH pathway antagonists that can be used to differentiate GTE cells into PP cells include, without limitation, Sant1 (CAS No. 304909-07-7, (Z)—N-(4-benzylpiperazin-1-yl)-1-(3,5-dimethyl-1-phenylpyrazol-4-yl)methanimine) and cyclopamine (CAS No. 4449-51-8, (3S,3′R,3′aS,6'S,6aS,6bS,7′aR,9R,11aS,11bR)-3′,6′,10,11b-tetramethylspiro[2,3,4,6,6a,6b,7,8,11,11a-decahydro-1H-benzo[a]fluorene-9,2′-3a,4,5,6,7,7a-hexahydro-3H-furo[3,2-b]pyridine]-3-ol). Examples of RA signaling pathway activators that can be used to differentiate GTE cells into PP cells include, without limitation, retinoic acid, TTNPB (CAS No. 71441-28-6, 4-[(E)-2-(5,6,7,8-tetrahydro-5,5,8,8-tetramethyl-2-naphthalenyl)-1-propenyl]benzoic acid), and DEAB (CAS No. 120-21-8, N,N-diethylaminobenzaldehyde).

As described herein, precursor cells (e.g., stem cells, DE cells, GTE cells, and PP cells) can be contacted with one or more agents (e.g., any described herein) for a period of time (e.g., about 1 to 14 days, such as from about 1 to 13 days, about 1 to 12 days, about 1 to 11 days, about 1 to 10 days, about 1 to 9 days, about 1 to 8 days, about 1 to 7 days, about 1 to 6 days, about 1 to 5 days, about 2 to 7 days, or about 2 to 5 days; or about 14, 13, 12, 11, 10, 9, 8, 7, 6, 5, 4, 3, 2, or 1 day(s)) sufficient to result in a population of desired cells.

Any appropriate amount of an agent (or a combination of agents) can be used to obtain a population of precursor cells. For example, between about 1 μM and about 3 μM (e.g., about 2 μM), between about 1 μM to about 50 μM (e.g., about 10 μM), between about 50 μM to about 300 μM (e.g., about 200 μM), between about 5 nM to about 50 μM (e.g., about 250 nM or about 500 nM; or from 5 nM to 25 μM, 5 nM to 10 μM, 5 nM to 5 μM, 5 nM to 4 μM, 5 nM to 3 μM, 5 nM to 2 μM, 5 nM to 1 μM, 5 nM to 500 nM, 5 nM to 250 nM, 5 nM to 100 nM, 10 nM to 50 μM, 10 nM to 25 μM, 10 nM to 10 μM, 10 nM to 5 μM, 10 nM to 4 μM, 10 nM to 3 μM, 10 nM to 2 μM, 10 nM to 1 μM, 10 nM to 500 nM, 10 nM to 250 nM, 10 nM to 100 nM, 15 nM to 50 μM, 15 nM to 25 μM, 15 nM to 10 μM, 15 nM to 5 μM, 15 nM to 4 μM, 15 nM to 3 μM, 15 nM to 2 μM, 15 nM to 1 μM, 15 nM to 500 nM, 15 nM to 250 nM, 15 nM to 100 nM, 20 nM to 50 μM, 20 nM to 25 μM, 20 nM to 10 μM, 20 nM to 5 μM, 20 nM to 4 μM, 20 nM to 3 μM, 20 nM to 2 μM, 20 nM to 1 μM, 20 nM to 500 nM, 20 nM to 250 nM, 20 nM to 100 nM, 25 nM to 50 μM, 25 nM to 25 μM, 25 nM to 10 μM, 25 nM to 5 μM, 25 nM to 4 μM, 25 nM to 3 μM, 25 nm to 2 μM, 25 nM to 1 μM, 25 nM to 500 nM, 25 nM to 250 nM, 25 nM to 100 nM, 30 nM to 50 μM, 30 nM to 25 μM, 30 nM to 10 μM, 30 nM to 5 μM, 30 nM to 4 μM, 30 nM to 3 μM, 30 nM to 2 μM, 30 nM to 1 μM, 30 nM to 500 nM, 30 nM to 250 nM, or 30 nM to 100 nM), between about 0.01 μM to about 250 μM (e.g., from 0.01 μM to 200 μM, 0.01 μM to 150 μM, 0.01 μM to 100 μM, 0.01 μM to 75 μM, 0.01 μM to 50 μM, 0.01 μM to 25 μM, 0.01 μM to 10 μM, 0.01 μM to 5 μM, 0.02 μM to 250 μM, 0.02 μM to 200 μM, 0.02 μM to 150 μM, 0.02 μM to 100 μM, 0.02 μM to 75 μM, 0.02 μM to 50 μM, 0.02 μM to 25 μM, 0.02 μM to 10 μM, 0.02 μM to 5 μM, 0.05 μM to 250 μM, 0.05 μM to 200 μM, 0.05 μM to 150 μM, 0.05 μM to 100 μM, 0.05 μM to 75 μM, 0.05 μM to 50 μM, 0.05 μM to 25 μM, 0.05 μM to 10 μM, 0.05 μM to 5 μM, 0.1 μM to 250 μM, 0.1 μM to 200 μM, 0.1 μM to 150 μM, 0.1 μM to 100 μM, 0.1 μM to 75 μM, 0.1 μM to 50 μM, 0.1 μM to 25 μM, 0.1 μM to 10 μM, 0.1 μM to 5 μM, 0.1 μM to 1 μM, 0.2 μM to 250 μM, 0.2 μM to 200 μM, 0.2 μM to 150 μM, 0.2 μM to 100 μM, 0.2 μM to 75 μM, 0.2 μM to 50 μM, 0.2 μM to 25 μM, 0.2 μM to 10 μM, 0.2 μM to 5 μM, 0.2 μM to 1 I, 0.5 μM to 250 μM, 0.5 μM to 200 μM, 0.5 μM to 150 μM, 0.5 μM to 100 μM, 0.5 μM to 75 μM, 0.5 μM to 50 μM, 0.5 μM to 25 μM, 0.5 μM to 10 μM, 0.5 μM to 5 μM, 1 μM to 250 μM, 1 μM to 200 μM, 1 μM to 150 μM, 1 μM to 100 μM, 1 μM to 75 μM, 1 μM to 50 μM, 1 μM to 25 μM, 1 μM to 10 μM, or 1 μM to 5 μM), or between about 25 ng/mL and about 200 ng/mL (e.g., about 50 ng/mL or about 100 ng/mL) of activin A, CHIR99021, KGF, PdBu, LDN193189, Sant1, and/or retinoic acid (RA) can be used together or in various combinations with culture medium to obtain a population of precursor cells described herein (e.g., a population of stem cells, DE cells, GTE cells, or PP cells). Such concentrations can be used with other compounds described herein (e.g., a growth factor from a TGF-β superfamily, a GSK3 inhibitor, a growth factor from an FGF family, a PKC activator, a BMP signaling pathway inhibitor, a SHH pathway antagonist, an RA signaling pathway activator, or a combination thereof).

In some cases, a population of precursor cells (e.g., a population of stem cells, DE cells, GTE cells, or PP cells) can be obtained and/or generated using methods and materials as described elsewhere (see, e.g., International PCT Patent Application Publication Nos. WO 2015/002724 and WO 2014/201167, Pagliuca et al., Cell, 159:428-439 (2014), and Peterson et al., Nat. Commun., 11:Article No. 2241 (14 pages) (2020)).

### Generation of Delta Progenitor (DP) Cells

Any appropriate method can be used to generate delta progenitor (DP) cells. In some cases, delta progenitor cells can be generated by differentiating PP cells into delta progenitor cells. For example, a fibroblast growth factor receptor (FGFR) inhibitor and a retinoic acid (RA) signaling pathway activator can be used to differentiate PP cells into delta progenitor cells. Examples of FGFR inhibitors that can be used to differentiate PP cells into delta progenitor cells include, without limitation, PD173074 (CAS No. 219580-11-7, 1-tert-butyl-3-[2-[4-(diethylamino)butylamino]-6-(3,5-dimethoxyphenyl)pyrido[2,3-d]pyrimidin-7-yl]urea), AZD4547 (CAS No. 1035270-39-3, N-[5-[2-(3,5-dimethoxyphenyl)ethyl]-1H-pyrazol-3-yl]-4-[(3S,5R)-3,5-dimethylpiperazin-1-yl]benzamide), erdafitinib (CAS No. 1346242-81-6, Catalog No. S8401 from Selleck Chemicals (Houston, TX), N′-(3,5-dimethoxyphenyl)-N′-[3-(1-methylpyrazol-4-yl)quinoxalin-6-yl]-N-propan-2-ylethane-1,2-diamine), roblitinib (CAS No. 1708971-55-4, Catalog No. S8548 from Selleck Chemicals, N-[5-cyano-4-(2-methoxyethylamino)pyridin-2-yl]-7-formyl-6-[(4-methyl-2-oxopiperazin-1-yl)methyl]-3,4-dihydro-2H-1,8-naphthyridine-1-carboxamide), pemigatinib (CAS No. 1513857-77-6, Catalog No. 33260 from Cayman Chemical, 11-(2,6-difluoro-3,5-dimethoxyphenyl)-13-ethyl-4-(morpholin-4-ylmethyl)-5,7,11,13-tetrazatricyclo[7.4.0.02,6]trideca-1,3,6,8-tetraen-12-one), and BGJ398 (Infigratinib) (CAS No. 872511-34-7, Catalog No. S2183 from Selleck Chemicals, 3-(2,6-dichloro-3,5-dimethoxyphenyl)-1-[6-[4-(4-ethylpiperazin-1-yl)anilino]pyrimidin-4-yl]-1-methylurea). Examples of RA signaling pathway activators that can be used to differentiate PP cells into delta progenitor cells include, without limitation, retinoic acid (including isomers thereof and salts thereof, such as CAS No. 302-79-4, 97950-17-9, or 13497-05-7), TTNPB (CAS No. 71441-28-6, 4-[(E)-2-(5,6,7,8-tetrahydro-5,5,8,8-tetramethyl-2-naphthalenyl)-1-propenyl]benzoic acid), and DEAB (CAS No. 120-21-8, N,N-diethylaminobenzaldehyde).

In some cases, PDX1⁺/NKX6.1⁻ PP cells can be used to generate delta progenitor cells. For example, PP cells such as PDX1⁺/NKX6.1⁻ PP cells can be contacted with one or more agents (e.g., one or more agents described herein) for a period of time (e.g., about 1 to 14 days, such as from about 1 to 13 days, about 1 to 12 days, about 1 to 11 days, about 1 to 10 days, about 1 to 9 days, about 1 to 8 days, about 1 to 7 days, about 1 to 6 days, about 1 to 5 days, about 2 to 7 days, or about 2 to 5 days; or about 14, 13, 12, 11, 10, 9, 8, 7, 6, 5, 4, 3, 2, or 1 day(s)) sufficient to result in a population of desired delta progenitor cells.

In some cases, a population of PP cells includes more than about 50%, 55%, 60%, 65%, 70%, 75%, 80%, 85%, 90%, 95%, 97%, or 99% Pdx1 positive cells. In some cases, the population of PP cells includes from about 50% to about 99% Pdx1 positive cells (e.g., from 50% to 97%, 50% to 95%, 50% to 90%, 50% to 85%, 50% to 80%, 50% to 75%, 50% to 70%, 50% to 65%, 50% to 60%, 55% to 99%, 55% to 97%, 55% to 95%, 55% to 90%, 55% to 85%, 55% to 80%, 55% to 75%, 55% to 70%, 55% to 65%, 55% to 60%, 60% to 99%, 60% to 97%, 60% to 95%, 60% to 90%, 60% to 85%, 60% to 80%, 60% to 75%, 60% to 70%, 60% to 65%, 65% to 99%, 65% to 97%, 65% to 95%, 65% to 90%, 65% to 85%, 65% to 80%, 65% to 75%, 65% to 70%, 70% to 99%, 70% to 97%, 70% to 95%, 70% to 90%, 70% to 85%, 70% to 80%, 70% to 75%, 75% to 99%, 75% to 97%, 75% to 95%, 75% to 90%, 75% to 85%, 75% to 80%, 80% to 99%, 80% to 97%, 80% to 95%, 80% to 90%, 80% to 85%, 85% to 99%, 85% to 97%, 85% to 95%, 85% to 90%, 90% to 99%, 90% to 97%, 90% to 95%, 95% to 99%, or 95% to 97%).

In some cases, a population of PP cells includes less than about 25%, 20%, 15%, 10%, 7%, 5%, 4%, 3%, 2%, 1%, 0.7%, 0.5%, 0.3%, or 0.1% Nkx6.1 positive cells. In some cases, the population of PP cells includes from 0% to about 25% Nkx6.1 positive cells (e.g., from 0% to 20%, 0% to 15%, 0% to 10%, 0% to 7%, 0% to 5%, 0% to 4%, 0% to 3%, 0% to 2%, 0% to 1%, 0% to 0.7%, 0% to 0.5%, 0% to 0.3%, 0% to 0.1%, and ranges therebetween).

Any appropriate amount of an agent (or combination of agents) can be used to differentiate PP cells (e.g., PDX1⁺/NKX6.1⁻ PP cells) into delta progenitor cells to obtain delta progenitor cells. For example, between about 1 μM and about 3 μM (e.g., about 2 μM), between about 1 μM to about 50 μM (e.g., about 10 μM), between about 50 μM to about 300 μM (e.g., about 200 μM), between about 5 nM to about 50 μM (e.g., about 250 nM or about 500 nM; or from 5 nM to 25 μM, 5 nM to 10 μM, 5 nM to 5 μM, 5 nM to 4 μM, 5 nM to 3 μM, 5 nM to 2 μM, 5 nM to 1 μM, 5 nM to 500 nM, 5 nM to 250 nM, 5 nM to 100 nM, 10 nM to 50 μM, 10 nM to 25 μM, 10 nM to 10 μM, 10 nM to 5 μM, 10 nM to 4 μM, 10 nM to 3 μM, 10 nM to 2 μM, 10 nM to 1 μM, 10 nM to 500 nM, 10 nM to 250 nM, 10 nM to 100 nM, 15 nM to 50 μM, 15 nM to 25 μM, 15 nM to 10 μM, 15 nM to 5 μM, 15 nM to 4 μM, 15 nM to 3 μM, 15 nM to 2 μM, 15 nM to 1 μM, 15 nM to 500 nM, 15 nM to 250 nM, 15 nM to 100 nM, 20 nM to 50 μM, 20 nM to 25 μM, 20 nM to 10 μM, 20 nM to 5 μM, 20 nM to 4 μM, 20 nM to 3 μM, 20 nM to 2 μM, 20 nM to 1 μM, 20 nM to 500 nM, 20 nM to 250 nM, 20 nM to 100 nM, 25 nM to 50 μM, 25 nM to 25 μM, 25 nM to 10 μM, 25 nM to 5 μM, 25 nM to 4 μM, 25 nM to 3 μM, 25 nM to 2 μM, 25 nM to 1 μM, 25 nM to 500 nM, 25 nM to 250 nM, 25 nM to 100 nM, 30 nM to 50 μM, 30 nM to 25 μM, 30 nM to 10 μM, 30 nM to 5 μM, 30 nM to 4 μM, 30 nM to 3 μM, 30 nM to 2 μM, 30 nM to 1 μM, 30 nM to 500 nM, 30 nM to 250 nM, or 30 nM to 100 nM), between about 0.01 μM to about 250 μM (e.g., from 0.01 μM to 200 μM, 0.01 μM to 150 μM, 0.01 μM to 100 μM, 0.01 μM to 75 μM, 0.01 μM to 50 μM, 0.01 μM to 25 μM, 0.01 μM to 10 μM, 0.01 μM to 5 μM, 0.02 μM to 250 μM, 0.02 μM to 200 μM, 0.02 μM to 150 μM, 0.02 μM to 100 μM, 0.02 μM to 75 μM, 0.02 μM to 50 μM, 0.02 μM to 25 μM, 0.02 μM to 10 μM, 0.02 μM to 5 μM, 0.05 μM to 250 μM, 0.05 μM to 200 μM, 0.05 μM to 150 μM, 0.05 μM to 100 μM, 0.05 μM to 75 μM, 0.05 μM to 50 μM, 0.05 μM to 25 μM, 0.05 μM to 10 μM, 0.05 μM to 5 μM, 0.1 μM to 250 μM, 0.1 μM to 200 μM, 0.1 μM to 150 μM, 0.1 μM to 100 μM, 0.1 μM to 75 μM, 0.1 μM to 50 μM, 0.1 μM to 25 μM, 0.1 μM to 10 μM, 0.1 μM to 5 μM, 0.1 μM to 1 μM, 0.2 μM to 250 μM, 0.2 μM to 200 μM, 0.2 μM to 150 μM, 0.2 μM to 100 μM, 0.2 μM to 75 μM, 0.2 μM to 50 μM, 0.2 μM to 25 μM, 0.2 μM to 10 μM, 0.2 μM to 5 μM, 0.2 μM to 1 μM, 0.5 μM to 250 μM, 0.5 μM to 200 μM, 0.5 μM to 150 μM, 0.5 μM to 100 μM, 0.5 μM to 75 μM, 0.5 μM to 50 μM, 0.5 μM to 25 μM, 0.5 μM to 10 μM, 0.5 μM to 5 μM, 1 μM to 250 μM, 1 μM to 200 μM, 1 μM to 150 μM, 1 μM to 100 μM, 1 μM to 75 μM, 1 μM to 50 μM, 1 μM to 25 μM, 1 μM to 10 μM, or 1 μM to 5 μM), or between about 25 ng/mL and about 200 ng/mL (e.g., about 50 ng/mL or about 100 ng/mL) of PD173074, erdafitinib, roblitinib, pemigatinib, BGJ398 (infigratinib), retinoic acid, TTNPB, and/or DEAB can be used together or in various combinations with culture medium to obtain delta progenitor cells. Such concentrations can be used with other compounds described herein (e.g., an FGFR inhibitor, a RA signaling pathway activator, or a combination thereof).

In some cases, one or more fibroblast growth factor receptor inhibitors are used at a concentration from about 5 nM to about 5 μM (e.g., about 250 nM or about 500 nM; or from 5 nM to 4 μM, 5 nM to 3 μM, 5 nM to 2 μM, 5 nM to 1 μM, 5 nM to 500 nM, 5 nM to 250 nM, 5 nM to 100 nM, 10 nM to 5 μM, 10 nM to 4 μM, 10 nM to 3 μM, 10 nM to 2 μM, 10 nM to 1 μM, 10 nM to 500 nM, 10 nM to 250 nM, 10 nM to 100 nM, 15 nM to 5 μM, 15 nM to 4 μM, 15 nM to 3 μM, 15 nM to 2 μM, 15 nM to 1 μM, 15 nM to 500 nM, 15 nM to 250 nM, 15 nM to 100 nM, 20 nM to 5 μM, 20 nM to 4 μM, 20 nM to 3 μM, 20 nM to 2 μM, 20 nM to 1 μM, 20 nM to 500 nM, 20 nM to 250 nM, 20 nM to 100 nM, 25 nM to 5 μM, 25 nM to 4 μM, 25 nM to 3 μM, 25 nM to 2 μM, 25 nM to 1 μM, 25 nM to 500 nM, 25 nM to 250 nM, 25 nM to 100 nM, 30 nM to 5 μM, 30 nM to 4 μM, 30 nM to 3 μM, 30 nM to 2 μM, 30 nM to 1 μM, 30 nM to 500 nM, 30 nM to 250 nM, or 30 nM to 100 nM) of PD173074, erdafitinib, roblitinib, pemigatinib, and/or BGJ398 (infigratinib). In some cases, the fibroblast growth factor receptor inhibitor is PD173074 at a concentration from about 5 nM to about 5 μM, in which ranges can be any described herein.

In some cases, one or more RA signaling pathway activators are used at a concentration from about 0.01 μM to about 250 μM (e.g., from 0.01 μM to 200 μM, 0.01 μM to 150 μM, 0.01 μM to 100 μM, 0.01 μM to 75 μM, 0.01 μM to 50 μM, 0.01 μM to 25 μM, 0.01 μM to 10 μM, 0.01 μM to 5 μM, 0.02 μM to 250 μM, 0.02 μM to 200 μM, 0.02 μM to 150 μM, 0.02 μM to 100 μM, 0.02 μM to 75 μM, 0.02 μM to 50 μM, 0.02 μM to 25 μM, 0.02 μM to 10 μM, 0.02 μM to 5 μM, 0.05 μM to 250 μM, 0.05 μM to 200 μM, 0.05 μM to 150 μM, 0.05 μM to 100 μM, 0.05 μM to 75 μM, 0.05 μM to 50 μM, 0.05 μM to 25 μM, 0.05 μM to 10 μM, 0.05 μM to 5 μM, 0.1 μM to 250 μM, 0.1 μM to 200 μM, 0.1 μM to 150 μM, 0.1 μM to 100 μM, 0.1 μM to 75 μM, 0.1 μM to 50 μM, 0.1 μM to 25 μM, 0.1 μM to 10 μM, 0.1 μM to 5 μM, 0.5 μM to 250 μM, 0.5 μM to 200 μM, 0.5 μM to 150 μM, 0.5 μM to 100 μM, 0.5 μM to 75 μM, 0.5 μM to 50 μM, 0.5 μM to 25 μM, 0.5 μM to 10 μM 0.5 μM to 5 μM, 1 μM to 250 μM, 1 μM to 200 μM, 1 μM to 150 μM, 1 μM to 100 μM, 1 μM to 75 μM, 1 μM to 50 μM, 1 μM to 25 μM, 1 μM to 10 μM, or 1 μM to 5 μM). In some cases, the RA signaling pathway activator is RA at a concentration from about 0.01 μM to about 250 μM, in which ranges can be any described herein.

In some cases, the population of delta progenitor cells can include a population including a plurality of somatostatin-positive cells or precursors thereof. In some cases, the population of delta progenitor cells can include a population including a plurality of glucagon-positive cells. In some cases, a population including the plurality of somatostatin-positive cells or precursors thereof is greater than a population including the plurality of glucagon-positive cells. In some cases, the population of delta progenitor cells can include a population including a plurality of insulin-positive cells.

A cell population can express one or more markers. In some cases, the one or more markers is selected from somatostatin, glucagon, insulin, or a combination thereof. In some cases, at least about 1%, 2%, 3%, 4%, 5%, 6%, 7%, 8%, 9%, 10%, 12%, 15%, 18%, or 20% of a cell population includes delta progenitor cells that express somatostatin. In some cases, about 1% to about 20% of a cell population includes delta progenitor cells that express somatostatin (e.g., from 1% to 15%, 1% to 10%, 1% to 5%, 2% to 20%, 2% to 15%, 2% to 10%, 2% to 5%, 3% to 20%, 3% to 15%, 3% to 10%, 3% to 5%, 4% to 20%, 4% to 15%, 4% to 10%, 4% to 5%, 5% to 20%, 5% to 15%, 5% to 10%, 7% to 20%, 7% to 15%, 7% to 10%, 10% to 20%, 10% to 15%, or 15% to 20%).

In some cases, about 10%, 7%, 5%, 4%, 3%, 2%, 1%, or less of a cell population includes delta progenitor cells that express glucagon. In some cases, 0% to about 15% of a cell population includes delta progenitor cells that express glucagon (e.g., from 0% to 10% or 0% to 5%).

In some cases, about 25%, 20%, 15%, 10%, 5%, or less of a cell population include delta progenitor cells that express insulin. In some cases, 0% to about 30% of a cell population includes delta progenitor cells that express insulin (e.g., from 0% to 25%, 0% to 20%, 0% to 15%, 0% to 10%, or 0% to 5%).

In some cases, the one or more markers is selected from Pdx1, CgA, or a combination thereof. In some cases, a cell population includes from about 1% to about 80% Pdx1 positive cells, CgA positive cells, or Pdx1 and CgA positive cells (e.g., from 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 2% to 80%, 2% to 75%, 2% to 70%, 2% to 65%, 2% to 60%, 2% to 55%, 2% to 50%, 2% to 45%, 2% to 40%, 2% to 35%, 2% to 30%, 2% to 25%, 2% to 20%, 3% to 80%, 3% to 75%, 3% to 70%, 3% to 65%, 3% to 60%, 3% to 55%, 3% to 50%, 3% to 45%, 3% to 40%, 3% to 35%, 3% to 30%, 3% to 25%, 3% to 20%, 4% to 80%, 4% to 75%, 4% to 70%, 4% to 65%, 4% to 60%, 4% to 55%, 4% to 50%, 4% to 45%, 4% to 40%, 4% to 35%, 4% to 30%, 4% to 25%, 4% to 20%, 5% to 80%, 5% to 75%, 5% to 70%, 5% to 65%, 5% to 60%, 5% to 55%, 5% to 50%, 5% to 45%, 5% to 40%, 5% to 35%, 5% to 30%, 5% to 25%, 5% to 20%, 7% to 80%, 7% to 75%, 7% to 70%, 7% to 65%, 7% to 60%, 7% to 55%, 7% to 50%, 7% to 45%, 7% to 40%, 7% to 35%, 7% to 30%, 7% to 25%, 7% to 20%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 15% to 80%, 15% to 75%, 15% to 70%, 15% to 65%, 15% to 60%, 15% to 55%, 15% to 50%, 15% to 45%, 15% to 40%, 15% to 35%, 15% to 30%, 15% to 25%, 15% to 20%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 25% to 80%, 25% to 75%, 25% to 70%, 25% to 65%, 25% to 60%, 25% to 55%, 25% to 50%, 25% to 45%, 25% to 40%, 25% to 35%, 25% to 30%, 30% to 80%, 30% to 75%, 30% to 70%, 30% to 65%, 30% to 60%, 30% to 55%, 30% to 50%, 30% to 45%, 30% to 40%, or 30% to 35%).

In some cases, a population of delta progenitor cells includes less than about 25%, 20%, 15%, 10%, 7%, 5%, 4%, 3%, 2%, 1%, 0.7%, 0.5%, 0.3%, or 0.1% Nkx6.1 positive cells. In some cases, the population of delta progenitor cells includes from 0% to about 25% Nkx6.1 positive cells (e.g., from 0% to 20%, 0% to 15%, 0% to 10%, 0% to 7%, 0% to 5%, 0% to 4%, 0% to 3%, 0% to 2%, 0% to 1%, 0% to 0.7%, 0% to 0.5%, 0% to 0.3%, 0% to 0.1%, and ranges therebetween).

A cell population can include delta progenitor cells in any amount. In some cases, a cell population can include at least about 1%, 2%, 3%, 4%, 5%, 6%, 7%, 8%, 9%, 10%, 15%, 20%, 25%, 30%, 35%, or 40% delta progenitor cells. In some cases, a cell population can include from about 1% to about 80% delta progenitor cells (e.g., from 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 2% to 80%, 2% to 75%, 2% to 70%, 2% to 65%, 2% to 60%, 2% to 55%, 2% to 50%, 2% to 45%, 2% to 40%, 2% to 35%, 2% to 30%, 2% to 25%, 2% to 20%, 3% to 80%, 3% to 75%, 3% to 70%, 3% to 65%, 3% to 60%, 3% to 55%, 3% to 50%, 3% to 45%, 3% to 40%, 3% to 35%, 3% to 30%, 3% to 25%, 3% to 20%, 4% to 80%, 4% to 75%, 4% to 70%, 4% to 65%, 4% to 60%, 4% to 55%, 4% to 50%, 4% to 45%, 4% to 40%, 4% to 35%, 4% to 30%, 4% to 25%, 4% to 20%, 5% to 80%, 5% to 75%, 5% to 70%, 5% to 65%, 5% to 60%, 5% to 55%, 5% to 50%, 5% to 45%, 5% to 40%, 5% to 35%, 5% to 30%, 5% to 25%, 5% to 20%, 7% to 80%, 7% to 75%, 7% to 70%, 7% to 65%, 7% to 60%, 7% to 55%, 7% to 50%, 7% to 45%, 7% to 40%, 7% to 35%, 7% to 30%, 7% to 25%, 7% to 20%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 15% to 80%, 15% to 75%, 15% to 70%, 15% to 65%, 15% to 60%, 15% to 55%, 15% to 50%, 15% to 45%, 15% to 40%, 15% to 35%, 15% to 30%, 15% to 25%, 15% to 20%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 25% to 80%, 25% to 75%, 25% to 70%, 25% to 65%, 25% to 60%, 25% to 55%, 25% to 50%, 25% to 45%, 25% to 40%, 25% to 35%, 25% to 30%, 30% to 80%, 30% to 75%, 30% to 70%, 30% to 65%, 30% to 60%, 30% to 55%, 30% to 50%, 30% to 45%, 30% to 40%, or 30% to 35%).

### Generation of Pre-Delta (PD) Cells

Any appropriate method can be used to generate pre-delta (PD) cells. In some cases, pre-delta cells can be generated by differentiating delta progenitor cells into pre-delta cells. For example, a TGF-β signaling inhibitor, or a combination of a PKC activator and a Notch signaling inhibitor, or a combination of a TGF-β signaling inhibitor, a PKC activator, and a Notch signaling inhibitor can be used to differentiate delta progenitor cells into pre-delta cells. Examples of TGF-β signaling inhibitors that can be used to differentiate delta progenitor cells into pre-delta cells include, without limitation, Alk5 inhibitor II (Alk5i, CAS No. 446859-33-2, 2-[5-(6-methylpyridin-2-yl)-1H-pyrazol-4-yl]-1,5-naphthyridine), SB431542 (CAS No. 301836-41-9, Catalog No. S1067 from Selleck Chemicals, 4-[4-(1,3-benzodioxol-5-yl)-5-pyridin-2-yl-1H-imidazol-2-yl]benzamide), and RepSox (CAS No. 446859-33-2, Catalog No. 72394 from STEMCELL Technologies Inc. (Vancouver, Canada), 2-[5-(6-methylpyridin-2-yl)-1H-pyrazol-4-yl]-1,5-naphthyridine). Examples of PKC activators that can be used to differentiate delta progenitor cells into pre-delta cells include, without limitation, indolactam V (CAS No. 90365-57-4, Catalog No. 14647 from Cayman Chemical, (10S,13S)-13-(hydroxymethyl)-9-methyl-10-propan-2-yl-3,9,12-triazatricyclo[6.6.1.0^{4,15}]pentadeca-1,4(15),5,7-tetraen-11-one), phorbol 12,13-dibutyrate (PdBu) (CAS No. 37558-16-0 or 61557-88-8, Catalog No. 4153 from Tocris Bioscience, (1aR,1bS,4aR,7aS,7bS,8R,9R,9aS)-1a,1b,4,4a,5,7a,7b,8,9,9a-decahydro-4a,7b-dihydroxy-3-(hydroxymethyl)-1,1,6,8-tetramethyl-5-oxo-1H-cyclopropa[3,4]benz[1,2-e]azulen-9,9a-diyl butanoic acid ester), and TPPB (CAS No. 497259-23-1, Catalog No. 5343 from Tocris Bioscience, (2E,4E)-N-[(2S,5S)-1,2,3,4,5,6-hexahydro-5-(hydroxymethyl)-1-methyl-2-(1-methylethyl)-3-oxo-1,4-benzodiazocin-8-yl]-5-[4-(trifluoromethyl)phenyl]-2,4-pentadienamide). Examples of Notch signaling inhibitors that can be used to differentiate delta progenitor cells into pre-delta cells include, without limitation, γ-secretase inhibitor XX (CAS No. 209984-56-5, N-[(1S)-2-[[(7S)-6,7-dihydro-5-methyl-6-oxo-5H-dibenz[b,d]azepin-7-yl]amino]-1-methyl-2-oxoethyl]-3,5-difluoro-benzeneacetamide), LY411575 (CAS No. 209984-57-6, Catalog No. S2714 from Selleck Chemical, (2S)-2-[[(2S)-2-(3,5-difluorophenyl)-2-hydroxyacetyl]amino]-N-[(7S)-5-methyl-6-oxo-7H-benzo[d][1]benzazepin-7-yl]propanamide), and Compound E (CAS No. 209986-17-4, Catalog No. 6476 from Tocris Bioscience, N-[(1S)-2-[[(3S)-2,3-dihydro-1-methyl-2-oxo-5-phenyl-1H-1,4-benzodiazepin-3-yl]amino]-1-methyl-2-oxoethyl]-3,5-difluorobenzeneacetamide).

In some cases, a BMP type 1 receptor inhibitor can be used to differentiate delta progenitor cells into pre-delta cells. Examples of BMP type 1 receptor inhibitors that can be used to differentiate delta progenitor cells into pre-delta cells include, without limitation, LDN193189.

In some cases, delta progenitor cells can be used to generate pre-delta cells. For example, delta progenitor cells can include any delta progenitor cells or cell population including delta progenitor cells that are described herein. In some cases, delta progenitor cells can include a population of a plurality of somatostatin-positive cells, a population of a plurality of glucagon-positive cells, a population of a plurality of insulin-positive cells, or a combination thereof.

Delta progenitor cells can be contacted with one or more agents (e.g., any described herein) for a period of time (e.g., about 1 to 14 days, such as from about 1 to 13 days, about 1 to 12 days, about 1 to 11 days, about 1 to 10 days, about 1 to 9 days, about 1 to 8 days, about 1 to 7 days, about 1 to 6 days, about 1 to 5 days, about 2 to 14 days, about 2 to 13 days, about 2 to 12 days, about 2 to 11 days, about 2 to 10 days, about 2 to 9 days, about 2 to 8 days, about 2 to 7 days, about 2 to 6 days, or about 2 to 5 days; or about 14, 13, 12, 11, 10, 9, 8, 7, 6, 5, 4, 3, 2, or 1 day(s)) sufficient to result in a population of desired pre-delta cells.

Any appropriate amount of an agent (or combination of agents) can be used to obtain pre-delta cells. For example, between about 1 μM and about 3 μM (e.g., about 2 μM), between about 1 μM to about 50 μM (e.g., about 10 μM), between about 50 μM to about 300 μM (e.g., about 200 μM), between about 5 nM to about 50 μM (e.g., about 250 nM or about 500 nM; or from 5 nM to 25 μM, 5 nM to 10 μM, 5 nM to 5 μM, 5 nM to 4 μM, 5 nM to 3 μM, 5 nM to 2 μM, 5 nM to 1 μM, 5 nM to 500 nM, 5 nM to 250 nM, 5 nM to 100 nM, 10 nM to 50 μM, 10 nM to 25 μM, 10 nM to 10 μM, 10 nM to 5 μM, 10 nM to 4 μM, 10 nM to 3 μM, 10 nM to 2 μM, 10 nM to 1 μM, 10 nM to 500 nM, 10 nM to 250 nM, 10 nM to 100 nM, 15 nM to 50 μM, 15 nM to 25 μM, 15 nM to 10 μM, 15 nM to 5 μM, 15 nM to 4 μM, 15 nM to 3 μM, 15 nM to 2 μM, 15 nM to 1 μM, 15 nM to 500 nM, 15 nM to 250 nM, 15 nM to 100 nM, 20 nM to 50 μM, 20 nM to 25 μM, 20 nM to 10 μM, 20 nM to 5 μM, 20 nM to 4 μM, 20 nM to 3 μM, 20 nM to 2 μM, 20 nM to 1 μM, 20 nM to 500 nM, 20 nM to 250 nM, 20 nM to 100 nM, 25 nM to 50 μM, 25 nM to 25 μM, 25 nM to 10 μM, 25 nM to 5 μM, 25 nM to 4 μM, 25 nM to 3 μM, 25 nM to 2 μM, 25 nM to 1 μM, 25 nM to 500 nM, 25 nM to 250 nM, 25 nM to 100 nM, 30 nM to 50 μM, 30 nM to 25 μM, 30 nM to 10 μM, 30 nM to 5 μM, 30 nM to 4 μM, 30 nM to 3 μM, 30 nM to 2 μM, 30 nM to 1 μM, 30 nM to 500 nM, 30 nM to 250 nM, or 30 nM to 100 nM), between about 0.01 μM to about 250 μM (e.g., from 0.01 μM to 200 μM, 0.01 μM to 150 μM, 0.01 μM to 100 μM, 0.01 μM to 75 μM, 0.01 μM to 50 μM, 0.01 μM to 25 μM, 0.01 μM to 10 μM, 0.01 μM to 5 μM, 0.02 μM to 250 μM, 0.02 μM to 200 μM, 0.02 μM to 150 μM, 0.02 μM to 100 μM, 0.02 μM to 75 μM, 0.02 μM to 50 μM, 0.02 μM to 25 μM, 0.02 μM to 10 μM, 0.02 μM to 5 μM, 0.05 μM to 250 μM, 0.05 μM to 200 μM, 0.05 μM to 150 μM, 0.05 μM to 100 μM, 0.05 μM to 75 μM, 0.05 μM to 50 μM, 0.05 μM to 25 μM, 0.05 μM to 10 μM, 0.05 μM to 5 μM, 0.1 μM to 250 μM, 0.1 μM to 200 μM, 0.1 μM to 150 μM, 0.1 μM to 100 μM, 0.1 μM to 75 μM, 0.1 μM to 50 μM, 0.1 μM to 25 μM, 0.1 μM to 10 μM 0.1 μM to 5 μM, 0.1 μM to 1 μM, 0.2 μM to 250 μM, 0.2 μM to 200 μM, 0.2 μM to 150 μM, 0.2 μM to 100 μM, 0.2 μM to 75 μM, 0.2 μM to 50 μM, 0.2 μM to 25 μM, 0.02 μM to 20 μM, 0.2 μM to 10 μM, 0.2 μM to 5 μM, 0.2 μM to 1 μM, 0.5 μM to 250 μM, 0.5 μM to 200 μM, 0.5 μM to 150 μM, 0.5 μM to 100 μM, 0.5 μM to 75 μM, 0.5 μM to 50 μM, 0.5 μM to 25 μM, 0.5 μM to 10 μM, 0.5 μM to 5 μM, 1 μM to 250 μM, 1 μM to 200 μM, 1 μM to 150 μM, 1 μM to 100 μM, 1 μM to 75 μM, 1 μM to 50 μM, 1 μM to 25 μM, 1 μM to 10 μM, or 1 μM to 5 μM), or between about 25 ng/mL and about 200 ng/mL (e.g., about 50 ng/mL or about 100 ng/mL) of Alk5 inhibitor II, SB431542, RepSox, indolactam V, γ-secretase inhibitor XX, and/or Compound E can be used together or in various combinations with culture medium to obtain pre-delta cells. Such concentrations can be used with other compounds described herein (e.g., a TGF-β signaling inhibitor, a PKC activator, a Notch signaling inhibitor, a BMP type 1 receptor inhibitor, or a combination thereof).

In some cases, one or more TGF-β signaling inhibitors are used at a concentration from about 0.01 μM to about 250 μM (e.g., from 0.01 μM to 200 μM, 0.01 μM to 150 μM, 0.01 μM to 100 μM, 0.01 μM to 75 μM, 0.01 μM to 50 μM, 0.01 μM to 25 μM, 0.01 μM to 10 μM, 0.01 μM to 5 μM, 0.02 μM to 250 μM, 0.02 μM to 200 μM, 0.02 μM to 150 μM, 0.02 μM to 100 μM, 0.02 μM to 75 μM, 0.02 μM to 50 μM, 0.02 μM to 25 μM, 0.02 μM to 20 μM, 0.02 μM to 10 μM, 0.02 μM to 5 μM, 0.05 μM to 250 μM, 0.05 μM to 200 μM, 0.05 μM to 150 μM, 0.05 μM to 100 μM, 0.05 μM to 75 μM, 0.05 μM to 50 μM, 0.05 μM to 25 μM, 0.05 μM to 10 μM, 0.05 μM to 5 μM, 0.1 μM to 250 μM, 0.1 μM to 200 μM, 0.1 μM to 150 μM, 0.1 μM to 100 μM, 0.1 μM to 75 μM, 0.1 μM to 50 V, 0.1 μM to 25 μM, 0.1 μM to 10 μM, 0.1 μM to 5 μM, 0.2 μM to 250 μM, 0.2 μM to 200 μM, 0.2 μM to 150 μM, 0.2 μM to 100 μM, 0.2 μM to 75 μM, 0.2 μM to 50 μM, 0.2 μM to 25 μM, 0.2 μM to 10 μM, 0.2 μM to 5 μM, 0.5 μM to 250 μM, 0.5 μM to 200 μM, 0.5 μM to 150 μM, 0.5 μM to 100 μM, 0.5 μM to 75 μM, 0.5 μM to 50 μM, 0.5 μM to 25 μM, 0.5 μM to 10 μM, 0.5 μM to 5 μM, 1 μM to 250 μM, 1 μM to 200 μM, 1 μM to 150 μM, 1 μM to 100 μM, 1 μM to 75 μM, 1 μM to 50 μM, 1 μM to 25 μM, 1 μM to 10 μM, or 1 μM to 5 μM). In some cases, the TGF-β signaling inhibitor is Alk5 inhibitor II at a concentration from about 0.01 μM to about 250 μM, in which ranges can be any described herein.

In some cases, one or more protein kinase C activators are used at a concentration from about 0.01 μM to about 250 μM (e.g., from 0.01 μM to 200 μM, 0.01 μM to 150 μM, 0.01 μM to 100 μM, 0.01 μM to 75 μM, 0.01 μM to 50 μM, 0.01 μM to 25 μM, 0.01 μM to 10 μM, 0.01 μM to 5 μM, 0.02 μM to 250 μM, 0.02 μM to 200 μM, 0.02 μM to 150 μM, 0.02 μM to 100 μM, 0.02 μM to 75 μM, 0.02 μM to 50 μM, 0.02 μM to 25 μM, 0.02 μM to 20 μM, 0.02 μM to 10 μM, 0.02 μM to 5 μM, 0.05 μM to 250 μM, 0.05 μM to 200 μM, 0.05 μM to 150 μM, 0.05 μM to 100 μM, 0.05 μM to 75 μM, 0.05 μM to 50 μM, 0.05 μM to 25 μM, 0.05 μM to 10 μM, 0.05 μM to 5 μM, 0.1 μM to 250 μM, 0.1 μM to 200 μM, 0.1 μM to 150 μM, 0.1 μM to 100 μM, 0.1 μM to 75 μM, 0.1 μM to 50 μM, 0.1 μM to 25 μM, 0.1 μM to 10 μM, 0.1 μM to 5 μM, 0.2 μM to 250 μM, 0.2 μM to 200 μM, 0.2 μM to 150 μM, 0.2 μM to 100 μM, 0.2 μM to 75 μM, 0.2 μM to 50 μM, 0.2 μM to 25 μM, 0.2 μM to 10 μM, 0.2 μM to 5 μM, 0.5 μM to 250 μM, 0.5 μM to 200 μM, 0.5 μM to 150 μM, 0.5 μM to 100 μM, 0.5 μM to 75 μM, 0.5 μM to 50 μM, 0.5 μM to 25 μM, 0.5 μM to 10 μM, 0.5 μM to 5 μM, 1 μM to 250 μM, 1 μM to 200 μM, 1 μM to 150 μM, 1 μM to 100 μM, 1 μM to 75 μM, 1 μM to 50 μM, 1 μM to 25 μM, 1 μM to 10 μM, or 1 μM to 5 μM). In some cases, the protein kinase C activator is indolactam V at a concentration from about 0.01 μM to about 250 μM, in which ranges can be any described herein.

In some cases, one or more Notch signaling inhibitors are used at a concentration from about 0.01 μM to about 250 μM (e.g., from 0.01 μM to 200 μM, 0.01 μM to 150 μM, 0.01 μM to 100 μM, 0.01 μM to 75 μM, 0.01 μM to 50 μM, 0.01 μM to 25 μM, 0.01 μM to 10 μM, 0.01 μM to 5 μM, 0.02 μM to 250 μM, 0.02 μM to 200 μM, 0.02 μM to 150 μM, 0.02 μM to 100 μM, 0.02 μM to 75 μM, 0.02 μM to 50 μM, 0.02 μM to 25 μM, 0.02 μM to 20 μM, 0.02 μM to 10 μM, 0.02 μM to 5 μM, 0.05 μM to 250 μM, 0.05 μM to 200 μM, 0.05 μM to 150 μM, 0.05 μM to 100 μM, 0.05 μM to 75 μM, 0.05 μM to 50 μM, 0.05 μM to 25 μM, 0.05 μM to 10 μM, 0.05 μM to 5 μM, 0.1 μM to 250 μM, 0.1 μM to 200 μM, 0.1 μM to 150 μM, 0.1 μM to 100 μM, 0.1 μM to 75 μM, 0.1 μM to 50 μM 0.1 μM to 25 μM, 0.1 μM to 10 μM, 0.1 μM to 5 μM, 0.2 μM to 250 μM, 0.2 μM to 200 μM, 0.2 μM to 150 μM, 0.2 μM to 100 μM, 0.2 μM to 75 μM, 0.2 μM to 50 μM, 0.2 μM to 25 μM 0.2 μM to 10 μM, 0.2 μM to 5 μM, 0.5 μM to 250 μM, 0.5 μM to 200 μM, 0.5 μM to 150 μM, 0.5 μM to 100 μM, 0.5 μM to 75 μM, 0.5 μM to 50 μM, 0.5 μM to 25 μM, 0.5 μM to 10 μM, 0.5 μM to 5 μM, 1 μM to 250 μM, 1 μM to 200 μM, 1 μM to 150 μM, 1 μM to 100 μM, 1 μM to 75 μM, 1 μM to 50 μM, 1 μM to 25 μM, 1 μM to 10 μM, or 1 μM to 5 μM). In some cases, the Notch signaling inhibitor is γ-secretase inhibitor XX at a concentration from about 0.01 μM to about 250 μM, in which ranges can be any described herein.

In some cases, the population of pre-delta cells can include a population including a plurality of somatostatin-positive cells or precursors thereof. In some cases, the population of pre-delta cells can include a population including a plurality of glucagon-positive cells. In some cases, a population including the plurality of somatostatin-positive cells or precursors thereof is greater than a population including the plurality of glucagon-positive cells. In some cases, the population of pre-delta cells can include a population including a plurality of insulin-positive cells.

A cell population can express one or more markers. In some cases, the one or more markers is selected from somatostatin, glucagon, insulin, or a combination thereof. In some cases, at least about 0.5%, 1%, 2%, 3%, 4%, or 5% of a cell population includes pre-delta cells that express somatostatin. In some cases, about 10%, 7%, 5%, 4%, 3%, 2%, 1%, or less of a cell population includes pre-delta cells that express somatostatin. In some cases, 0% to about 50% of a cell population includes pre-delta cells that express somatostatin (e.g., from 0% to 45%, 0% to 40%, 0% to 35%, 0% to 30%, 0% to 25%, 0% to 20%, 0% to 15%, 0% to 10%, 0% to 5%, 0% to 3%, 0.5% to 50%, 0.5% to 45%, 0.5% to 40%, 0.5% to 35%, 0.5% to 30%, 0.5% to 25%, 0.5% to 20%, 0.5% to 15%, 0.5% to 10%, 0.5% to 5%, 0.5% to 3%, 1% to 50%, 1% to 45%, 1% to 40%, 1% to 35%, 1% to 30%, 1% to 25%, 1% to 20%, 1% to 15%, 1% to 10%, 1% to 5%, 1% to 3%, 2% to 50%, 2% to 45%, 2% to 40%, 2% to 35%, 2% to 30%, 2% to 25%, 2% to 20%, 2% to 15%, 2% to 10%, 2% to 5%, 2% to 3%, 5% to 50%, 5% to 45%, 5% to 40%, 5% to 35%, 5% to 30%, 5% to 25%, 5% to 20%, 5% to 15%, 5% to 10%, 10% to 50%, 10% to 45%, 10% to 40%, 10% to 35%, 10% to 30%, 10% to 25%, 10% to 20%, 10% to 15%, 15% to 50%, 15% to 45%, 15% to 40%, 15% to 35%, 15% to 30%, 15% to 25%, 15% to 20%, 20% to 50%, 20% to 45%, 20% to 40%, 20% to 35%, 20% to 30%, or 20% to 25%).

In some cases, at least about 0.5%, 1%, 2%, 3%, 4%, 5%, 10%, 15%, 20%, 25%, 30%, or 35% of a cell population includes pre-delta cells that express somatostatin but do not express glucagon and insulin. In some cases, about 1% to about 50% of a cell population includes pre-delta cells that express somatostatin but do not express glucagon and insulin (e.g., from 1% to 45%, 1% to 40%, 1% to 35%, 1% to 30%, 1% to 25%, 1% to 20%, 1% to 15%, 1% to 10%, 1% to 5%, 1% to 3%, 2% to 50%, 2% to 45%, 2% to 40%, 2% to 35%, 2% to 30%, 2% to 25%, 2% to 20%, 2% to 15%, 2% to 10%, 2% to 5%, 2% to 3%, 5% to 50%, 5% to 45%, 5% to 40%, 5% to 35%, 5% to 30%, 5% to 25%, 5% to 20%, 5% to 15%, 5% to 10%, 10% to 50%, 10% to 45%, 10% to 40%, 10% to 35%, 10% to 30%, 10% to 25%, 10% to 20%, 10% to 15%, 15% to 50%, 15% to 45%, 15% to 40%, 15% to 35%, 15% to 30%, 15% to 25%, 15% to 20%, 20% to 50%, 20% to 45%, 20% to 40%, 20% to 35%, 20% to 30%, or 20% to 25%).

In some cases, at least 25%, 20%, 15%, 10%, 7%, 5%, 4%, 3%, 2%, 1%, or less of a cell population includes pre-delta cells that express glucagon. In some cases, 0% to about 30% of a cell population includes pre-delta cells that express glucagon (e.g., from 0% to 25%, 0% to 20%, 0% to 15%, 0% to 10%, or 0% to 5%).

In some cases, at least 20%, 15%, 10%, 5%, 4%, 3%, 2%, 1%, or less of a cell population include pre-delta cells that express insulin. In some cases, 0% to about 25% of a cell population includes pre-delta cells that express insulin (e.g., from 0% to 20%, 0% to 15%, 0% to 10%, or 0% to 5%).

In some cases, the one or more markers is selected from Sst, Pdx1, PC2, CgA, CgB, Pax6, Hhex, Ptch1, or a combination thereof. In some cases, a cell population includes from about 1% to about 40% Sst positive cells, Pdx1 positive cells, PC2 positive cells, CgA positive cells, CgB positive cells, Pax6 positive cells, Hhex positive cells, Ptch1 positive cells, or a combination of Sst, Pdx1, PC2, CgA, CgB, Pax6, Hhex, or Ptch1 positive cells (e.g., from 1% to 35%, 1% to 30%, 1% to 25%, 1% to 20%, 1% to 10%, 2% to 40%, 2% to 35%, 2% to 30%, 2% to 25%, 2% to 20%, 2% to 10%, 5% to 40%, 5% to 35%, 5% to 30%, 5% to 25%, 5% to 20%, 5% to 10%, 10% to 40%, 10% to 35%, 10% to 30%, 10% to 25%, 10% to 20%, 15% to 40%, 15% to 35%, 15% to 30%, 15% to 25%, 15% to 20%, 20% to 40%, 20% to 35%, 20% to 30%, or 20% to 25%). In some cases, the one or more markers is selected from Sst, Pdx1, CgA, Pax6, or a combination thereof. In some cases, the one or more markers is selected from Pdx1, PC2, CgA, CgB, Pax6, Hhex, Ptch1, or a combination thereof. In some cases, the one or more markers is selected from Pdx1, CgA, Pax6, Hhex, Ptch1, or a combination thereof. In some cases, the one or more markers is selected from Pdx1, Pax6, Hhex, Ptch1, or a combination thereof.

In some cases, a population of pre-delta cells includes less than about 25%, 20%, 15%, 10%, 7%, 5%, 4%, 3%, 2%, 1%, 0.7%, 0.5%, 0.3%, or 0.1% Nkx6.1 positive cells. In some cases, the population of pre-delta cells includes from 0% to about 25% Nkx6.1 positive cells (e.g., from 0% to 20%, 0% to 15%, 0% to 10%, 0% to 7%, 0% to 5%, 0% to 4%, 0% to 3%, 0% to 2%, 0% to 1%, 0% to 0.7%, 0% to 0.5%, 0% to 0.3%, 0% to 0.1%, and ranges therebetween).

In some cases, a population of pre-delta cells includes less than about 25%, 20%, 15%, 10%, 7%, 5%, 4%, 3%, 2%, 1%, 0.7%, 0.5%, 0.3%, or 0.1% Arx positive cells. In some cases, the population of pre-delta cells includes from 0% to about 25% Arx positive cells (e.g., from 0% to 20%, 0% to 15%, 0% to 10%, 0% to 7%, 0% to 5%, 0% to 4%, 0% to 3%, 0% to 2%, 0% to 1%, 0% to 0.7%, 0% to 0.5%, 0% to 0.3%, 0% to 0.1%, and ranges therebetween).

In some cases, a cell population includes a population of SC-delta cells, SC-alpha cells, SC-beta cells, non-hormonal cells, polyhormonal cells, or a combination thereof. In some cases, a population of SC-alpha cells is present in an amount that is greater than a population of SC-beta cells. In some cases, about 60%, 50%, 40%, 30%, 20%, or less of a cell population of pre-delta cells includes a population of polyhormonal cells. In some cases, a cell population includes about 10% to 60% of a population of polyhormonal cells (e.g., from 10% to 55%, 10% to 50%, 10% to 45%, 10% to 40%, 10% to 35%, 10% to 30%, 10% to 25%, 10% to 20%, 20% to 60%, 20% to 55%, 20% to 50%, 20% to 45%, 20% to 40%, 20% to 35%, 20% to 30%, or 20% to 25%).

A cell population can include pre-delta cells in any amount. In some cases, about 10%, 7%, 5%, 4%, 3%, 2%, 1%, or less of a cell population includes pre-delta cells. In some cases, a cell population can include at least about 1%, 2%, 3%, 4%, 5%, 6%, 7%, 8%, 9%, 10%, 15%, 20%, 25%, 30%, 35%, or 40% pre-delta cells. In some cases, a cell population can include from about 1% to about 40% pre-delta cells (e.g., from 1% to 35%, 1% to 30%, 1% to 25%, 1% to 20%, 1% to 10%, 2% to 40%, 2% to 35%, 2% to 30%, 2% to 25%, 2% to 20%, 2% to 10%, 5% to 40%, 5% to 35%, 5% to 30%, 5% to 25%, 5% to 20%, 5% to 10%, 10% to 40%, 10% to 35%, 10% to 30%, 10% to 25%, 10% to 20%, 15% to 40%, 15% to 35%, 15% to 30%, 15% to 25%, 15% to 20%, 20% to 40%, 20% to 35%, 20% to 30%, or 20% to 25%).

### Generation of Stem Cell-Derived Delta (SC-Delta) Cells

Any appropriate method can be used to generate stem cell-derived delta (SC-delta) cells. In some cases, SC-delta cells can be generated by differentiating pre-delta cells into SC-delta cells. For example, an adenylyl cyclase activator can be used to differentiate pre-delta cells into SC-delta cells. Examples of adenylyl cyclase activators that can be used to differentiate pre-delta cells into SC-delta cells include, without limitation, forskolin (CAS No. 66575-29-9 or 66428-89-5, Catalog No. 11018 from Cayman Chemical, [(3R,4aR,5S,6S,6aS,10S,10aR,10bS)-3-ethenyl-6,10,10b-trihydroxy-3,4a,7,7,10a-pentamethyl-1-oxo-5,6,6a,8,9,10-hexahydro-2H-benzo[f]chromen-5-yl]acetate), NKH 477 (CAS No. 138605-00-2, Catalog No. 1603 from Tocris Bioscience, colforsin daropate hydrochloride, [(3R,4aR,5S,6S,6aS,10S,10aR,10bS)-5-acetyloxy-3-ethenyl-10,10b-dihydroxy-3,4a,7,7,10a-pentamethyl-1-oxo-5,6,6a,8,9,10-hexahydro-2H-benzo[f]chromen-6-yl]3-(dimethylamino)propanoate; hydrochloride), PACAP 1-27 (Pituitary Adenylate Cyclase-Activating Polypeptide 1-27, Catalog No. 1183 from Tocris Bioscience, HSDGIFTDSYSRYRKQMAVKKYLAAVL (with a modification at Leu-27 having a C-terminal amide), SEQ ID NO:25), or PACAP 1-38 (Pituitary Adenylate Cyclase-Activating Polypeptide 1-38, Catalog No. 1186 from Tocris Bioscience, HSDGIFTDSYSRYRKQMAVKKYLAAVLGKRYKQRVKNK (with a modifications at Lys-38 having a C-terminal amide), SEQ ID NO:26).

In some cases, pre-delta cells can be exposed to one or more of the following to generate SC-delta cells: an adenylyl cyclase activator (e.g., forskolin, CAS No. 66575-29-9 or 66428-89-5, Catalog No. 11018 from Cayman Chemical, [(3R,4aR,5S,6S,6aS,10S,10aR,10bS)-3-ethenyl-6,10,10b-trihydroxy-3,4a,7,7,10a-pentamethyl-1-oxo-5,6,6a,8,9,10-hexahydro-2H-benzo[f]chromen-5-yl]acetate)); an apoptosis inhibitor (e.g., genipin, CAS No. 6902-77-8, Catalog No. 078-03021 from FUJIFILM Wako Chemicals U.S.A. Corporation (Richmond, VA), methyl (1R,4aS,7aS)-1-hydroxy-7-(hydroxymethyl)-1,4a,5,7a-tetrahydrocyclopenta[c]pyran-4-carboxylate); a selective class IIA histone deacetylase inhibitor (e.g., TMP269, CAS No. 1314890-29-3, N-[[4-(4-phenyl-1,3-thiazol-2-yl)oxan-4-yl]methyl]-3-[5-(trifluoromethyl)-1,2,4-oxadiazol-3-yl]benzamide); a cytokinin or an analog thereof (e.g., 6-benzylaminopurine riboside, CAS No. 4294-16-0, Catalog No. AC226422500 from Acros Organics (Thermo Scientific Chemicals, Waltham, MA), (2R,3R,4S,5R)-2-[6-(benzylamino)purin-9-yl]-5-(hydroxymethyl)oxolane-3,4-diol; kinetin (CAS No. 525-79-1); or kinetin riboside (CAS No. 4338-47-0)); a selective inhibitor of platelet-derived growth factor receptor (PDGFR) such as PDGFR α/β (e.g., CP-673451, CAS No. 343787-29-1, Catalog No. S1536 from Selleck Chemicals, (1-[2-[5-(2-methoxyethoxy) benzimidazole-1-yl]quinolin-8-yl]piperidin-4-amine)); an acetyltransferase p300 (KAT3B) inhibitor (e.g., L002, CAS No. 321695-57-2, Catalog No. 5045 from Tocris Bioscience, ([(3,5-dimethyl-4-oxocyclohexa-2,5-dien-1-ylidene)amino]4-methoxybenzenesulfonate)); and/or an extracellular signal-regulated kinase 1 (ERK1) inhibitor (e.g., SC1 (pluripotin, CAS No. 839707-37-8, Catalog No. SC-255607 from Santa Cruz Biotechnology, Inc. (Dallas, TX), N-(3-(7-(1,3-dimethyl-1H-pyrazol-5-ylamino)-1-methyl-2-oxo-1,2-dihydropyrimido[4,5-d]pyrimidin-3(4H)-yl)-4-methylphenyl)-3-(trifluoromethyl)benzamide)).

In some cases, pre-delta cells can be used to generate stem cell-derived delta cells. For example, pre-delta cells can include any pre-delta cells or cell population including pre-delta cells that are described herein. In some cases, pre-delta cells can include a population of a plurality of somatostatin-positive cells, a population of a plurality of glucagon-positive cells, a population of a plurality of insulin-positive cells, or a combination thereof.

Pre-delta cells can be contacted with one or more agents (e.g., any described herein) for a period of time (e.g., about 1 to 45 days, 1 to 30 days, 2 to 45 days, 2 to 30 days, 1 to 14 days, such as from about 1 to 13 days, about 1 to 12 days, about 1 to 11 days, about 1 to 10 days, about 1 to 9 days, about 1 to 8 days, about 1 to 7 days, about 1 to 6 days, about 1 to 5 days, about 2 to 7 days, or about 2 to 5 days; or about 35, 30, 28, 24, 21, 20, 14, 13, 12, 11, 10, 9, 8, 7, 6, 5, 4, 3, 2, or 1 day(s)) sufficient to result in a population of desired SC-delta cells.

Any appropriate amount of an agent (or combination of agents) can be used to obtain SC-delta cells. For example, between about 1 μM and about 3 μM (e.g., about 2 μM), between about 1 μM to about 50 μM (e.g., about 10 μM), between about 50 μM to about 300 μM (e.g., about 200 μM), between about 5 nM to about 50 μM (e.g., about 250 nM or about 500 nM; or from 5 nM to 25 μM, 5 nM to 10 μM, 5 nM to 5 μM, 5 nM to 4 μM, 5 nM to 3 μM, 5 nM to 2 μM, 5 nM to 1 μM, 5 nM to 500 nM, 5 nM to 250 nM, 5 nM to 100 nM, 10 nM to 50 μM, 10 nM to 25 μM, 10 nM to 10 μM, 10 nM to 5 μM, 10 nM to 4 μM, 10 nM to 3 μM, 10 nM to 2 μM, 10 nM to 1 μM, 10 nM to 500 nM, 10 nM to 250 nM, 10 nM to 100 nM, 15 nM to 50 μM, 15 nM to 25 μM, 15 nM to 10 μM, 15 nM to 5 μM, 15 nM to 4 μM, 15 nM to 3 μM, 15 nM to 2 μM, 15 nM to 1 μM, 15 nM to 500 nM, 15 nM to 250 nM, 15 nM to 100 nM, 20 nM to 50 μM, 20 nM to 25 μM, 20 nM to 10 μM, 20 nM to 5 μM, 20 nM to 4 μM, 20 nM to 3 μM, 20 nM to 2 μM, 20 nM to 1 μM, 20 nM to 500 nM, 20 nM to 250 nM, 20 nM to 100 nM, 25 nM to 50 μM, 25 nM to 25 μM, 25 nM to 10 μM, 25 nM to 5 μM, 25 nM to 4 μM, 25 nM to 3 μM, 25 nM to 2 μM, 25 nM to 1 μM, 25 nM to 500 nM, 25 nM to 250 nM, 25 nM to 100 nM, 30 nM to 50 μM, 30 nM to 25 μM, 30 nM to 10 μM, 30 nM to 5 μM, 30 nM to 4 μM, 30 nM to 3 μM, 30 nM to 2 μM, 30 nM to 1 μM, 30 nM to 500 nM, 30 nM to 250 nM, or 30 nM to 100 nM), between about 0.01 μM to about 250 μM (e.g., from 0.01 μM to 200 μM, 0.01 μM to 150 μM, 0.01 μM to 100 μM, 0.01 μM to 75 μM, 0.01 μM to 50 μM, 0.01 μM to 25 μM, 0.01 μM to 10 μM, 0.01 μM to 5 μM, 0.02 μM to 250 μM, 0.02 μM to 200 μM, 0.02 μM to 150 μM, 0.02 μM to 100 μM, 0.02 μM to 75 μM, 0.02 μM to 50 μM, 0.02 μM to 25 μM, 0.02 μM to 10 μM, 0.02 μM to 5 μM, 0.05 μM to 250 μM, 0.05 μM to 200 μM, 0.05 μM to 150 μM, 0.05 μM to 100 μM, 0.05 μM to 75 μM, 0.05 μM to 50 μM, 0.05 μM to 25 μM, 0.05 μM to 10 μM, 0.05 μM to 5 μM, 0.1 μM to 250 μM, 0.1 μM to 200 μM, 0.1 μM to 150 μM, 0.1 μM to 100 μM, 0.1 μM to 75 μM, 0.1 μM to 50 μM, 0.1 μM to 25 μM, 0.1 μM to 10 μM, 0.1 μM to 5 μM, 0.1 μM to 1 μM, 0.2 μM to 250 μM, 0.2 μM to 200 μM, 0.2 μM to 150 μM, 0.2 μM to 100 μM, 0.2 μM to 75 μM, 0.2 μM to 50 μM, 0.2 μM to 25 μM, 0.2 μM to 10 μM, 0.2 μM to 5 μM, 0.2 μM to 1 μM, 0.5 μM to 250 μM, 0.5 μM to 200 μM, 0.5 μM to 150 μM, 0.5 μM to 100 μM, 0.5 μM to 75 μM, 0.5 μM to 50 μM, 0.5 μM to 25 μM, 0.5 μM to 10 μM, 0.5 μM to 5 μM, 1 μM to 250 μM, 1 μM to 200 μM, 1 μM to 150 μM, 1 μM to 100 μM, 1 μM to 75 μM, 1 μM to 50 μM, 1 μM to 25 μM, 1 μM to 10 μM, or 1 μM to 5 μM), between about 0.5 μM to about 750 μM (e.g., from 0.5 μM to 700 μM, 0.5 μM to 600 μM, 0.5 μM to 500 μM, 0.5 μM to 400 μM, 0.5 μM to 300 μM, 0.5 μM to 200 μM, 0.5 μM to 100 μM, 0.5 μM to 50 μM, 0.5 μM to 10 μM, 1 μM to 750 μM, 1 μM to 700 μM, 1 μM to 600 μM, 1 μM to 500 μM, 1 μM to 400 μM, 1 μM to 300 μM, 1 μM to 200 μM, 1 μM to 100 μM, 1 μM to 50 μM, 1 μM to 10 μM, 2 μM to 750 μM, 2 μM to 700 μM, 2 μM to 600 μM, 2 μM to 500 μM, 2 μM to 400 μM, 2 μM to 300 μM, 2 μM to 200 μM, 2 μM to 100 μM, 2 μM to 50 μM, 2 μM to 10 μM, 3 μM to 750 μM, 3 μM to 700 μM, 3 μM to 600 μM, 3 μM to 500 μM, 3 μM to 400 μM, 3 μM to 300 μM, 3 μM to 200 μM, 3 μM to 100 μM, 3 μM to 50 μM, 3 μM to 10 μM, 5 μM to 750 μM, 5 μM to 700 μM, 5 μM to 600 μM, 5 μM to 500 μM, 5 μM to 400 μM, 5 μM to 300 μM, 5 μM to 200 μM, 5 μM to 100 μM, 5 μM to 50 μM, or 5 μM to 10 μM), or between about 25 ng/mL and about 200 ng/mL (e.g., about 50 ng/mL or about 100 ng/mL) of forskolin or a derivative thereof (e.g., a salt thereof or a water-soluble derivative thereof), NKH 477, PACAP 1-27, and/or PACAP 1-38 can be used together or in various combinations with culture medium to obtain SC-delta cells. Such concentrations can be used with other compounds described herein (e.g., an adenylyl cyclase activator, an apoptosis inhibitor, a selective class IIA histone deacetylase inhibitor, a cytokinin or an analog thereof, a selective inhibitor of PDGFR, a KAT3B inhibitor, an ERK1 inhibitor, or a combination thereof).

In some cases, one or more adenylyl cyclase activators are used at a concentration from about 0.5 μM to about 750 μM (e.g., from 0.5 μM to 700 μM, 0.5 μM to 600 μM, 0.5 μM to 500 μM, 0.5 μM to 400 μM, 0.5 μM to 300 μM, 0.5 μM to 200 μM, 0.5 μM to 100 μM, 0.5 μM to 50 μM, 0.5 μM to 10 μM, 1 μM to 750 μM, 1 μM to 700 μM, 1 μM to 600 μM, 1 μM to 500 μM, 1 μM to 400 μM, 1 μM to 300 μM, 1 μM to 200 μM, 1 μM to 100 μM, 1 μM to 50 μM, 1 μM to 10 μM, 2 μM to 750 μM, 2 μM to 700 μM, 2 μM to 600 μM, 2 μM to 500 μM, 2 μM to 400 μM, 2 μM to 300 μM, 2 μM to 200 μM, 2 μM to 100 μM, 2 μM to 50 μM, 2 μM to 10 μM, 3 μM to 750 μM, 3 μM to 700 μM, 3 μM to 600 μM, 3 μM to 500 μM, 3 μM to 400 μM, 3 μM to 300 μM, 3 μM to 200 μM, 3 μM to 100 μM, 3 μM to 50 μM, 3 μM to 10 μM, 5 μM to 750 μM, 5 μM to 700 μM, 5 μM to 600 μM, 5 μM to 500 μM, 5 μM to 400 μM, 5 μM to 300 μM, 5 μM to 200 μM, 5 μM to 100 μM, 5 μM to 50 μM, or 5 μM to 10 μM). In some cases, the adenylyl cyclase activator is forskolin at a concentration from about 0.5 μM to about 750 μM, in which ranges can be any described herein.

In some cases, the population of SC-delta cells can include a population including a plurality of somatostatin-positive cells or precursors thereof. In some cases, the population of SC-delta cells can include a population including a plurality of glucagon-positive cells. In some cases, a population including the plurality of somatostatin-positive cells or precursors thereof is greater than a population including the plurality of glucagon-positive cells. In some cases, the population of SC-delta cells can include a population including a plurality of insulin-positive cells.

A cell population can express one or more markers. In some cases, the one or more markers is selected from somatostatin, glucagon, insulin, or a combination thereof. In some cases, at least about 0.5%, 1%, 2%, 3%, 4%, 5%, 1%, %15%, 20%, 25%, 30%, or 35% of a cell population includes SC-delta cells that express somatostatin. In some cases, 0% to about 90% of a cell population includes cells (e.g., SC-delta cells in optional combination with other cells) that express somatostatin (e.g., from 0% to 85%, 0% to 80%, 0% to 75%, 0% to 70%, 0% to 65%, 0% to 60%, 0% to 55%, 0% to 50%, 0% to 45%, 0% to 40%, 0% to 35%, 0% to 30%, 0% to 25%, 0% to 20%, 0% to 15%, 0% to 10%, 0% to 5%, 0% to 3%, 1% to 90%, 1% to 85%, 1% to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 1% to 3%, 5% to 90%, 5% to 85%, 5% to 80%, 5% to 75%, 5% to 70%, 5% to 65%, 5% to 60%, 5% to 55%, 5% to 50%, 5% to 45%, 5% to 40%, 5% to 35%, 5% to 30%, 5% to 25%, 5% to 20%, 5% to 15%, 5% to 10%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 40% to 90%, 40% to 85%, 40% to 80%, 40% to 75%, 40% to 70%, 40% to 65%, 40% to 60%, 40% to 55%, 40% to 50%, 40% to 45%, 50% to 90%, 50% to 85%, 50% to 80%, 50% to 75%, 50% to 70%, 50% to 65%, 50% to 60%, or 50% to 55%).

In some cases, at least about 0.5%, 1%, 2%, 3%, 4%, 5% 10%, 15%, 20%, 25%, 30%, or 35% of a cell population includes SC-delta cells that express somatostatin but do not express glucagon and insulin. In some cases, about 1% to about 50% of a cell population includes SC-delta cells that express somatostatin but do not express glucagon and insulin (e.g., from 1% to 45%, 1% to 40%, 1% to 35%, 1% to 30%, 1% to 25%, 1% to 20%, 1% to 15%, 1% to 10%, 1% to 5%, 1% to 3%, 2% to 50%, 2% to 45%, 2% to 40%, 2% to 35%, 2% to 30%, 2% to 25%, 2% to 20%, 2% to 15%, 2% to 10%, 2% to 5%, 2% to 3%, 5% to 50%, 5% to 45%, 5% to 40%, 5% to 35%, 5% to 30%, 5% to 25%, 5% to 20%, 5% to 15%, 5% to 10%, 10% to 50%, 10% to 45%, 10% to 40%, 10% to 35%, 10% to 30%, 10% to 25%, 10% to 20%, 10% to 15%, 15% to 50%, 15% to 45%, 15% to 40%, 15% to 35%, 15% to 30%, 15% to 25%, 15% to 20%, 20% to 50%, 20% to 45%, 20% to 40%, 20% to 35%, 20% to 30%, or 20% to 25%).

In some cases, about 30% 25%, 20%, 15%, 10%, 7% 5%, 4%, 3%, 2%, 1%, or less of a cell population includes SC-delta cells that express glucagon. In some cases, 0% to about 40% of a cell population includes SC-delta cells that express glucagon (e.g., from 0% to 35%, 0% to 30%, 0% to 25%, 0% to 20%, 0% to 15%, 0% to 10%, 0% to 5%, 1% to 40%, 1% to 35%, 1% to 30%, 1% to 25%, 1% to 20%, 1% to 15%, 1% to 10%, 1% to 5%, 3% to 40%, 3% to 35%, 3% to 30%, 3% to 25%, 3% to 20%, 3% to 15%, 3% to 10%, 3% to 5%, 5% to 40%, 5% to 35%, 5% to 30%, 5% to 25%, 5% to 20%, 5% to 15%, or 5% to 10%).

In some cases, about 60%, 55%, 50%, 45%, 40%, 35%, 30%, 25%, 20%, 15%, 10%, 5%, 4%, 3%, 2%, 1%, or less of a cell population includes SC-delta cells that express insulin. In some cases, 0% to about 50% of a cell population includes SC-delta cells that express insulin (e.g., from 0% to 45%, 0% to 40%, 0% to 35%, 0% to 30%, 0% to 25%, 0% to 20%, 0% to 15%, 0% to 10%, 0% to 5%, 1% to 50%, 1% to 45%, 1% to 40%, 1% to 35%, 1% to 30%, 1% to 25%, 1% to 20%, 1% to 15%, 1% to 10%, 1% to 5%, 3% to 50%, 3% to 45%, 3% to 40%, 3% to 35%, 3% to 30%, 3% to 25%, 3% to 20%, 3% to 15%, 3% to 10%, 3% to 5%, 5% to 50%, 5% to 45%, 5% to 40%, 5% to 35%, 5% to 30%, 5% to 25%, 5% to 20%, 5% to 15%, 5% to 10%, 10% to 50%, 10% to 45%, 10% to 40%, 10% to 35%, 10% to 30%, 10% to 25%, 10% to 20%, or 10% to 15%).

In some cases, the one or more markers is selected from Sst, Pdx1, PC2, CgA, CgB, Pax6, Hhex, Ptch1, or a combination thereof. In some cases, a cell population includes from about 1% to about 40% Sst positive cells, Pdx1 positive cells, PC2 positive cells, CgA positive cells, CgB positive cells, Pax6 positive cells, Hhex positive cells, Ptch1 positive cells, or a combination of Sst, Pdx1, CgA, Pax6, Hhex, or Ptch1 positive cells (e.g., from 1% to 35%, 1% to 30%, 1% to 25%, 1% to 20%, 1% to 10%, 2% to 40%, 2% to 35%, 2% to 30%, 2% to 25%, 2% to 20%, 2% to 10%, 5% to 40%, 5% to 35%, 5% to 30%, 5% to 25%, 5% to 20%, 5% to 10%, 10% to 40%, 10% to 35%, 10% to 30%, 10% to 25%, 10% to 20%, 15% to 40%, 15% to 35%, 15% to 30%, 15% to 25%, 15% to 20%, 20% to 40%, 20% to 35%, 20% to 30%, or 20% to 25%). In some cases, the one or more markers is selected from Sst, Pdx1, PC2, CgA, Pax6, or a combination thereof. In some cases, the one or more markers is selected from Sst, Pdx1, CgA, Pax6, or a combination thereof. In some cases, the one or more markers is selected from Pdx1, CgA, Pax6, Hhex, Ptch1, or a combination thereof. In some cases, the one or more markers is selected from PC2, CgA, CgB, Pax6, Hhex, Ptch1, or a combination thereof. In some cases, the one or more markers is selected from Pax6, Hhex, Ptch1, or a combination thereof.

In some cases, a population of SC-delta cells includes less than about 25%, 20%, 15%, 10%, 7%, 5%, 4%, 3%, 2%, 1%, 0.7%, 0.5%, 0.3%, or 0.1% Nkx6.1 positive cells. In some cases, the population of SC-delta cells includes from 0% to about 25% Nkx6.1 positive cells (e.g., from 0% to 20%, 0% to 15%, 0% to 10%, 0% to 7%, 0% to 5%, 0% to 4%, 0% to 3%, 0% to 2%, 0% to 1%, 0% to 0.7%, 0% to 0.5%, 0% to 0.3%, 0% to 0.1%, and ranges therebetween).

In some cases, a population of SC-delta cells includes less than about 25%, 20%, 15%, 10%, 7%, 5%, 4%, 3%, 2%, 1%, 0.7%, 0.5%, 0.3%, or 0.1% Arx positive cells. In some cases, the population of SC-delta cells includes from 0% to about 25% Arx positive cells (e.g., from 0% to 20%, 0% to 15%, 0% to 10%, 0% to 7%, 0% to 5%, 0% to 4%, 0% to 3%, 0% to 2%, 0% to 1%, 0% to 0.7%, 0% to 0.5%, 0% to 0.3%, 0% to 0.1%, and ranges therebetween).

In some cases, a population of SC-delta cells includes less than about 25%, 20%, 15%, 10%, 7%, 5%, 4%, 3%, 2%, 1%, 0.7%, 0.5%, 0.3%, or 0.1% Gcg positive cells. In some cases, the population of SC-delta cells includes from 0% to about 25% Gcg positive cells (e.g., from 0% to 20%, 0% to 15%, 0% to 10%, 0% to 7%, 0% to 5%, 0% to 4%, 0% to 3%, 0% to 2%, 0% to 1%, 0% to 0.7%, 0% to 0.5%, 0% to 0.3%, 0% to 0.1%, and ranges therebetween).

In some cases, a population of SC-delta cells includes less than about 25%, 20%, 15%, 10%, 7%, 5%, 4%, 3%, 2%, 1%, 0.7%, 0.5%, 0.3%, or 0.1% Ins positive cells. In some cases, the population of SC-delta cells includes from 0% to about 25% Ins positive cells (e.g., from 0% to 20%, 0% to 15%, 0% to 10%, 0% to 7%, 0% to 5%, 0% to 4%, 0% to 3%, 0% to 2%, 0% to 1%, 0% to 0.7%, 0% to 0.5%, 0% to 0.3%, 0% to 0.1%, and ranges therebetween).

In some cases, a cell population includes a population of SC-delta cells, SC-alpha cells, SC-beta cells, non-hormonal cells, polyhormonal cells, or a combination thereof. In some cases, a population of SC-alpha cells is present in an amount that is greater than a population of SC-beta cells. In some cases, about 60%, 50%, 40%, 30%, 20%, or less of a cell population of SC-delta cells includes a population of polyhormonal cells. In some cases, a cell population includes about 10% to 60% of a population of polyhormonal cells (e.g., from 10% to 55%, 10% to 50%, 10% to 45%, 10% to 40%, 10% to 35%, 10% to 30%, 10% to 25%, 10% to 20%, 20% to 60%, 20% to 55%, 20% to 50%, 20% to 45%, 20% to 40%, 20% to 35%, 20% to 30%, or 20% to 25%).

A cell population can include SC-delta cells in any amount. In some cases, a cell population can include at least about 1%, 2%, 3%, 4%, 5%, 6%, 7%, 8%, 9%, 10%, 15%, 20%, 25%, 30%, 35%, 40%, 50%, or 60% SC-delta cells. In some cases, a cell population can include from about 1% to about 60% SC-delta cells (e.g., from 1% to 55%, 1% to 50%, 1% to 45%, 1% to 40%, 1% to 35%, 1% to 30%, 1% to 25%, 1% to 20%, 1% to 10%, 2% to 60%, 2% to 55%, 2% to 50%, 2% to 45%, 2% to 40%, 2% to 35%, 2% to 30%, 2% to 25%, 2% to 20%, 2% to 10%, 5% to 60%, 5% to 55%, 5% to 50%, 5% to 45%, 5% to 40%, 5% to 35%, 5% to 30%, 5% to 25%, 5% to 20%, 5% to 10%, 10% to 60%, 10% to 55%, 10% to 50%, 10% to 45%, 10% to 40%, 10% to 35%, 10% to 30%, 10% to 25%, 10% to 20%, 15% to 60%, 15% to 55%, 15% to 50%, 15% to 45%, 15% to 40%, 15% to 35%, 15% to 30%, 15% to 25%, 15% to 20%, 20% to 60%, 20% to 55%, 20% to 50%, 20% to 45%, 20% to 40%, 20% to 35%, 20% to 30%, or 20% to 25%).

The invention will be further described in the following examples, which do not limit the scope of the invention described in the claims.

### EXAMPLES

### Example 1: In Vitro Generation of Pancreatic Delta Cells from Human Pluripotent Stem Cells

The human pancreas contains an exocrine system that helps in digestion and an endocrine system that controls blood glucose. Within the endocrine system, there are islets of Langerhans composed of different endocrine and non-endocrine cell types. Although insulin secreting β (beta) cells and glucagon secreting a (alpha) cells are the two major endocrine cell types that control glucose flux through counter regulation, somatostatin secreting δ (delta) cells, pancreatic polypeptide secreting γ (gamma) cells, and ghrelin secreting ε (epsilon) cells also coordinate in final islets hormone output. The factors secreted by beta cells such as insulin, Zn²⁺, ATP, serotonin, and γ-aminobutyric acid (GABA) can suppress the activity of alpha cells, whereas the factor acetylcholine secreted by alpha cells can suppress the activity of beta cells. Delta cells respond to factors such as urocortin 3, acetylcholine, glutamate, and ghrelin released by other endocrine cells and secrete somatostatin. Hormone somatostatin fine-tunes the activity of beta and alpha cells and inhibits secretion of both insulin and glucagon, thus also acts as a regulator of glucose homeostasis. Glucagon stimulates somatostatin secretion via activation of glucagon and GLP1 receptors both at hypo- and hyperglycemic conditions, however, the precise role of insulin and its receptors is inconclusive due to varied results observed in different species. Therefore, it is unlikely that glucagon stimulation of somatostatin is indirectly mediated through glucagon induced insulin secretion from beta cells. Additionally, delta cells can reach and control a large number of beta cells through filopodia and alpha cells through gap junctions. The regulatory function of delta cells on alpha and beta cells to coordinate precise pancreatic islet hormone output requires them to be active in a wide physiological range of glucose concentration (e.g., from 3-20 mM). Ablation of delta cells from islets can result in increased insulin secretion in response to glucose. Somatostatin secretion is also triggered by epsilon cells through ghrelin receptors (GHSRs) on delta cells to exert negative feedback on beta and alpha cells. Thus, intra-islet communication can play a major role in final islets hormone output, and any insult can cause disturbed glucose homeostasis leading to pathophysiological condition such as diabetes.

Although beta cell dysfunction is the primary focus in diabetes, the functional role of other cells can be equally involved. For instance, in Type 1 diabetes, the loss of counter-regulatory stimulation of glucagon secretion in response to exogenous insulin-induced hypoglycemia may be due to either alpha cell dysfunction or an increased somatostatin secretion by dysfunctional delta cell under hypoglycemic condition. Nevertheless, it is also interesting to understand the function of individual pancreatic endocrine cells in the absence of beta cells as in Type 1 diabetes, since these cells not only depend on glucose but also on beta cell secretory factors for their function. Type 1 diabetes results from autoimmune destruction of beta cells, whereas Type 2 diabetes may result from either dysfunctional endocrine cells or due to the loss of sensitivity of tissues like liver, adipose and skeletal muscle to endocrine hormones. Although Type 1 diabetes could potentially be treated through transplantation of stem cell derived beta (SC-β) cells, the treatment efficacy could be improved to that of cadaveric islets containing multiple endocrine cell types by combining SC-β cells with other stem cell derived pancreatic endocrine cells. But this approach can be limited due to the lack of effective methods for generation of other endocrine cell types. To this end, the generation of functional pancreatic endocrine cell types has been proposed as a means of reconstituting the endocrine pancreas.

Previous reports include the development of protocols capable of generating functional pancreatic beta cells and alpha cells in vitro from several pluripotent stem cell sources (e.g., hES and iPS) (see, e.g., Pagliuca et al., Cell, 159:428-439 (2014) and Peterson et al., Nat. Commun., 11:Article No. 2241 (14 pages) (2020)). These cells are capable of secreting insulin and/or glucagon in response to glucose both in vitro and in vivo. Similar findings were also reported elsewhere (see, e.g., Rezania et al., Nat. Biotechnol., 32:1121-1133 (2014); and Russ et al., EMBO J., 34:1759-1772 (2015)); and a feature in these protocols is the use of chemical and growth factor signals that are thought to mimic those present in the developing pancreas. Although this approach is promising, lack of complete knowledge on complex signals that govern formation of different endocrine cell subtypes in vivo has hindered the production of fully functional endocrine cells in vitro. Described herein is the use of a combinatorial screening strategy to identify small molecules capable of differentiating human embryonic stem cells (hESCs) into delta cells (SC-δ) that express markers of pancreatic delta cells and secrete somatostatin upon glucose challenge.

### Example 2: Non-Limiting Methods and Materials

### Cell Culture

The human embryonic stem cell line HUES8 was used for all experiments described herein. The cells were cultured in mTeSR (STEMCELL Technologies, Vancouver, Canada) media in a suspension-based 3D culture system using 500 mL spinner flask (Corning, VWR). The flasks were maintained at a rotation rate of 70 rpm in humidified incubator set at 37° C., and 5% CO₂. The cells were adapted to 3D culture by seeding 150 million cells in mTeSR media along with 10 μM Y27632. After 48 hours of culture, the flasks were fed with fresh mTeSR without Y27632, and cells were passaged every 72 hours by dispersing the clusters into single cells by Accutase and seeded into fresh mTeSR with Y27632.

### Directed Differentiation

After 72 hours of culture in mTeSR, the clusters of HUES8 cells were allowed to settle at the bottom of the spinner flask, and spent media was replaced with stage specific differentiation media containing specific growth factors and small molecules. The compositions of stage specific media (S1, S2, and S3) are described elsewhere (see, e.g., Pagliuca et al., Cell, 159:428-439 (2014)), and the additional details of growth factors can be found in Table 1, which shows factor concentrations used during stage 1 through stage 6 of differentiation.

TABLE 1
Factor concentrations used during stage 1 through stage 6 of differentiation

| | Final | | | |
|---|---|---|---|---|
| Chemical name | concentration | | Target/Action | Vendor and Catalog number |
| Activin A | 100 | ng/mL | Smad activation and regulation of | R&D Systems, 338-AC |
| | | | activin-responsive gene | |
| | | | transcription | |
| Chirr-99021 (Chir) | 3 | μM | GSK-3α/β inhibitor | TOCRIS, 4423 |
| Keratinocyte Growth Factor (KGF) | 50 | ng/mL | FGFR2b agonist | PEPROTECH, 100-19 |
| Sant-1 | 250 | nM | Hedgehog (Hh) signaling inhibitor | R&D Systems, 1974 |
| Phorbol-12,13-dibutyrate (PDBu) | 500 | nM | PKC Activator | Millipore-Sigma, 524390 |
| Retinoic Acid (RA) | 2 | μM | RAR a, b, g agonist | Sigma Aldrich, R2625 |
| LDN193189 Hydrochloride (LDN) | 200 | nM | BMP Type 1 receptor inhibitor | Sigma Aldrich, SML0559 |
| Alk5 inhibitor II (Alk5i) | 10 | μM | TGF-β/Activin signaling inhibitor | DNSK, DNSK-ALK5-02 |
| γ-secretase inhibitor XX (DBZ) | 2 | μM | Notch signaling inhibitor | Cayman Chemical Company, |
| | | | | 14627 |
| (—)-Indolactam V | 2 | μM | PKC Activator | Cayman Chemical Company, |
| | | | | 14647 |
| Forskolin | 200 | μM | Adenylyl Cyclase activation | Cayman Chemical Company, |

11018

In brief, the following media were used:
S1 media: MCDB 131 (Cellgro; 15-100-CV)+8 mM D-(+)-Glucose (Sigma; G7528)+2.46 g/L NaHCO₃ (Sigma; S3817)+2% (w/v) FAF-BSA (fatty acid-free bovine serum albumin; Proliant; 68700)+ITS-X (Insulin-Transferrin-Selenium-Ethanolamine; Invitrogen; 51500056) 1:50,000+2 mM Glutamax (L-alanyl-L-glutamine dipeptide; Invitrogen; 35050079)+0.25 mM Vitamin C (Sigma Aldrich; A4544)+1% (v/v) Pen/Strep (Penicillin/Streptomycin; Cellgro; 30-002-CT).
S2 media: MCDB 131+8 mM D-(+)-Glucose+1.23 g/L NaHCO₃+2% (w/v) FAF-BSA+ITS-X 1:50,000+2 mM Glutamax+0.25 mM Vitamin C+1% (v/v) Pen/Strep.
S3 media: MCDB 131+8 mM D-(+)-Glucose+1.23 g/L NaHCO₃+2% (w/v) FAF-BSA+ITS-X 1:200+2 mM Glutamax+0.25 mM Vitamin C+1% (v/v) Pen/Strep.

The differentiation was carried out in six stages. The generation of PP1 cells from HUES8 cells was carried out using prior protocols with modifications (see, e.g., Pagliuca et al., Cell, 159:428-439 (2014); and Peterson et al., Nat. Commun., 11:Article No. 2241 (14 pages) (2020)); and the delta cell differentiation was further carried out using PP1 cells. The media changes for differentiation were as follows:

$$Day 1 ( Stage 1 ) \text{:} S 1 + 100 ng / mL Activin A + 3 μ M CHIR 99021.$$

$$Day 2 ( Stage 1 ) \text{:} S 1 + 100 ng / mL Activin A .$$

$$Day 4 ( Stage 2 ) \text{:} S 2 + 50 ng / mL KGF .$$

$$Day 6 ( Stage 3 ) \text{:} S 3 + 50 ng / mL KGF + 250 nM Sant 1 + 500 nM PdBu + 2 μ M RA + 200 nM LDN 193189.$$

$$Day 7 \text{:} S 3 + 50 ng / mL KGF + 250 nM Sant 1 + 500 nM PdBU + 2 μ M RA .$$

$$Day 8 , 10 , 12 ( Stage 4 ) \text{:} S 3 + 200 nM PD 173074 + 2 μ M RA .$$

$$Day 13 , 15 ( Stage 5 ) \text{:} S 3 + 10 μ M Alk 5 i + 2 μ M Indolactam V + 2 μ M γ - secretase inhibitor XX .$$

$$Day 17 , 19 , 21 ( Stage 6 ) \text{:} S 3 + 200 μ M Forskolin .$$

The differentiation was terminated on Day 23 of the protocol. The progression of differentiation was assessed at different stages during the course of differentiation by collecting clusters of cells.

### Flow Cytometry

The cell clusters collected during the course of differentiation at different stages were dissociated into single cell suspension by treating with TrypLE Express at 37° C. for 10 minutes after washing twice with phosphate buffered saline (PBS). The enzyme activity was quenched by adding equal volume of S3 media to prevent further degradation, and dispersed single cells were washed with PBS, fixed in 4% paraformaldehyde (PFA) for 20 minutes and subsequently stored at 4° C. until further use. On the day of staining, the cells were passed through 40 μm filter and washed twice with PBS before permeabilizing in block solution (PBS+0.1% Triton X-100+5% donkey serum; PBST). After 40 minutes of blocking, the cells were incubated with primary antibodies in block solution for 1 hour at room temperature (RT), washed twice with PBST, and then incubated with appropriate secondary antibodies in block solution for 1 hour at RT. Finally, cells were washed twice with PBST and resuspended in PBS at a concentration of 1×10⁶ cells/mL. The stained cells were acquired in Attune flow cytometer and were analyzed using FlowJo v10 software. The Pdx1 (R&D Systems, AF2419) antibody was used at 1:250, Nkx6.1 (Iowa Hybridoma Bank, F55A12) at 1:100, insulin (Iowa Hybridoma Bank, GN-ID4) at 1:250, glucagon (LSBio, LS-B10219) at 1:25,000, and somatostatin (Santa Cruz Biotechnologies, SC-25262) at a dilution of 1:8000. The secondary antibodies are either donkey-anti-goat-Alexa Fluor 488 (Invitrogen, A-11055, 1:1000), donkey-anti-rabbit-Alexa Fluor 488 (Invitrogen, A-21206, 1:1000), donkey-anti-rat-Alexa Fluor 594 (Invitrogen, A-21209, 1:1000), or donkey-anti-mouse-Alexa Fluor 647 (Invitrogen, A-31571 1:1000). The list of primary antibodies is presented in Table 3.

TABLE 3
List of primary antibodies used in flow cytometry and immunohistochemistry

| | | Catalog | | Dilution | Dilution |
|---|---|---|---|---|---|
| Antibody | Company | number | Species | (Flow cytometry) | (Immunohistochemistry) |
| PDX1 | R & D Systems | AF419 | Goat | 1:250 | 1:100 |
| NKX6.1 | Iowa Hybridoma | F55A12 | Mouse | 1:100 | 1:100 |
| | Bank | | | | |
| PAX6 | Biolegend | 901301 | Rabbit | — | 1:100 |
| HHEX | R & D Systems | MAB83771 | Rabbit | — | 1:500 |
| PTCH1 | LSbio | LS-B3939 | Goat | — | 1:4000 |
| ARX | R & D Systems | AF7068 | Sheep | — | 1:1000 |
| CRHR2 | LSbio | LS-B9473 | Rabbit | — | 1:500 |
| RBP4 | Invitrogen | PA5-29173 | Rabbit | — | 1:250 |
| Somatostatin | Santa Cruz | SC-25262 | Mouse | 1:5000 | 1:100 |
| | Biotechnology | | | | |
| Somatostatin | Novus | NBP1-87022 | Rabbit | — | 1:200 |
| | Biologicals | | | | |
| Somatostatin | American | 13-2366 | Sheep | — | 1:1000 |
| | Research | | | | |
| | Products | | | | |
| Insulin | Iowa Hybridoma | GN-ID4 | Mouse | 1:250 | 1:250 |
| | Bank | | | | |
| Glucagon | LSbio | LS-B10219 | Rabbit | 1:25000 | 1:25000 |
| Glucagon | Sigma-Aldrich | G2654 | Mouse | — | 1:1000 |
| Chromogranin A | Abcam | ab15160 | Rabbit | — | 1:5000 |
| Chromogranin B | Proteintech | 14968-I-AP | Rabbit | — | 1:2000 |
| PC1/3 | Abcam | ab3532 | Rabbit | — | 1:200 |
| PC2 | R & D Systems | MAB6018 | Mouse | — | 1:100 |

The gating strategy used for flow plots is shown in FIG. 18A.

### Immunohistochemistry

The differentiated cell clusters or human islets were fixed in 4% PFA and stored at 4° C. until further use. The clusters were washed three times with PBS, set in Histogel (Thermo), embedded in paraffin, and sectioned at 4 μm for histological analysis. Antigen retrieval was performed in citrate buffer (pH 6.5) for 1 hour in boiling water bath on deparaffinized and rehydrated sections, and then allowed to cool for 40 minutes. Sections were then permeabilized and blocked in block solution (PBS+0.1% Triton X-100+5% donkey serum; PBST) for 40 minutes. Primary antibodies were added at appropriate dilutions and incubated overnight at 4° C. On the following day, secondary antibodies were added for 1 hour at room temperature. After thorough rinse with PBST, sections were mounted in Vectashield (Vector Laboratories), covered with coverslips, and sealed with nail polish. The sections were visualized using Zeiss microscope. The detailed information of primary antibodies is listed in Table 3. The secondary antibodies are either donkey-anti-goat-Alexa Fluor 488 (Invitrogen, A-11055, 1:1000), donkey-anti-rabbit-Alexa Fluor 488 (Invitrogen, A-21206, 1:1000), donkey-anti-rat-Alexa Fluor 594 (Invitrogen, A-21209, 1:1000), or donkey-anti-mouse-Alexa Fluor 647 (Invitrogen, A-31571 1:1000).

### High-Content Screen

Clusters were collected from either Stage 3, or Stage 4, or Stage 5 completed flasks, washed three times with PBS, dispersed into single cells using TrypLE Express for 10 minutes, and quenched using S3 media. The single cells were then seeded into matrigel (Corning, 356231) coated 384-well plates at a seeding density of 50,000 cells per well. Chemical compounds were then introduced to each well and incubated at 37° C. in a humidified atmosphere. The fresh media and compounds were added every other day by replacing the spent media. After 5 days for Stage 3 completed cells, 4 days for Stage 4 completed cells, and 7 days for Stage 5 completed cells of incubation, the cells were fixed with 4% PFA and stained for insulin, glucagon, and somatostatin as described herein. Stained cells were imaged with the Cellomics Scan Version 6.6.0 (Thermo Scientific HCS Studio). Twenty-five view fields were captured for each well and analyzed. The percentage of cells expressing only somatostatin was calculated for each well.

### TMR Red Assay

Terminal deoxynucleotidyl transferase dUTP nick end labeling (TUNEL) assay was performed using an In Situ Cell Death TMR red Detection Kit from Roche (12156792910). Deparaffinized, rehydrated, and antigen retrieved paraffin sections were permeabilized and blocked as described herein. A total of 50 μL freshly prepared TUNEL reaction mixture is added for 60 minutes at 37° C. in a humidified atmosphere in the dark. Sections were rinsed thoroughly with PBS, treated with suitable primary and secondary antibodies at appropriate dilutions, and visualized under microscope as described herein. For evaluation of TMR red, an excitation wavelength in the range of 520-560 nm (maximum 540 nm; green) and detection in the range of 570-620 nm (maximum 580 nm; red) were used.

### Western Blotting

Protein lysates were prepared from HUES8 and differentiated clusters collected at different stages using cell lysis buffer (10 mM Tris-HCl [pH 8.0], 10 mM NaCl, and 0.5% NP-40) containing protease inhibitor (Roche) by incubating on ice for 30 minutes. Lysates were centrifuged at 12,000×g for 20 minutes at 4° C., and concentration of protein was determined in the supernatant using Microplate BCA protein Assay kit (Pierce Biotechnology). A total of 8 μg of each protein sample was separated by 20% sodium dodecyl sulfate-polyacrylamide gel electrophoresis (SDS-PAGE), and Western blotting was carried out using antibodies against somatostatin (1:1000) and α-actin (1:1000). Different band sizes were determined by running a protein ladder (BIORAD, 1610374).

### Transmission Electron Microscopy

HUES8 and differentiated clusters stored in TRUMPS buffer at 4° C. were processed by the Microscopy and Cell Analysis Core, Electron Microscopy Laboratory at Mayo Clinic, Rochester, MN. Grids were analyzed for the ultrastructure of granules under electron microscope; representative images were captured with camera.

### In Vitro Functional Studies

Sequential glucose stimulated insulin secretion (GSIS), glucose stimulated somatostatin secretion (GSSS), and glucose stimulated glucagon secretion (GSGS) were performed on human islets (Prodo Laboratories) or differentiated SC-δ cells to measure secreted insulin, somatostatin, and glucagon hormones. For GSIS and GSSS, the cells were fasted in low-glucose (3.3 mM) Krebs Ringer Buffer (KRB) (128 mM NaCl, 5 mM KCl, 2.7 mM CaCl₂, 1.2 mM MgSO₄, 1 mM Na₂HPO₄, 1.2 mM KH₂PO₄, 5 mM NaHCO₃, 10 mM HEPES (4-(2-hydroxyethyl)-1-piperazineethanesulfonic acid), and 0.1% BSA (bovine serum albumin) in Milli-Q water). Whereas for GSGS, the cells were fasted in high-glucose KRB. Clusters collected from differentiation flask and were washed once with PBS and fasting buffer before transferring into 24-well transwell inserts. The clusters were fasted in humidified incubator for 1 hour at 37° C. in either low- or high-glucose KRB. Clusters were washed once in either low- or high-glucose KRB, and then transferred to low-glucose KRB for 1 hour at 37° C. Clusters were then transferred to high-glucose KRB (16.5 mM) for 1 hour at 37° C. Of note, for GSGS, clusters were washed additionally during transfer from low-glucose KRB to high-glucose KRB. Clusters were finally transferred to low-glucose KRB containing a depolarizing agent KCl (30 mM) for 1 hour at 37° C. Clusters were then dispersed into single cells by treating with TrypLE express for 20 minutes, and cells were counted using Countess II FL (Thermo). During each transfer, the supernatant was collected and stored at −20° C. until further use. The concentrations of secreted hormones insulin (ALPCO, 80-INSHUU-E10) and glucagon (R&D Systems, DY1249 & DY008) and somatostatin (Phoenix Pharmaceuticals, EK-060-03) were analyzed by ELISA, somatostatin was additionally analyzed by RIA (DIAsource, RB306RUO) and normalized to cell number.

RNA Extraction, Quantitative Real-Time PCR (qRT-PCR)

The differentiating clusters and human cadaveric islets procured from Prodo Labs were washed with PBS to remove medium, aspirated, and immediately stored at −80° C. until further use. Total RNA was extracted using RNeasy® Micro Kit (Qiagen, Valencia, CA), quantified by NanoDrop (ThermoFisher, Waltham, MA), and cDNA was synthesized from 400 ng of total RNA by using iScript™ cDNA Synthesis Kit (Bio-Rad, Hercules, CA). qRT-PCR was performed on the LightCycler®96 (Roche). Data were analyzed using the 2^{-ΔΔCT} method with mRNA levels normalized to GAPDH. The list of primers used is provided in Table 2.

TABLE 2
List of primers used in qRT-PCR

| | | SEQ | | SEQ |
|---|---|---|---|---|
| Gene | Forward primer | ID NO: | Reverse primer | ID NO: |
| PDX1 | CGTCCAGCTGCCTTTCCCAT | 1 | CCGTGAGATGTACTTGTTGA | 2 |
| | | | AAGGA | |
| NKX6.1 | CCGAGTCCTGCTTCTTCTTG | 3 | ATTCGTTGGGGATGACAGAG | 4 |
| PAX6 | AACCAATTCCACAACCCACC | 5 | TTATTTGCCATGGTGAAGCT | 6 |
| | ACAC | | GGGC | |
| HHEX | CATGTTCAGAAAACTGGATT | 7 | CCTAAGAGCAGTACATAAAC | 8 |
| | TAGGAATAATGT | | TATTTGTTAAGTC | |
| PTCH1 | GTTGTGGGCCTCCTCATATT | 9 | GACTTACTCGTCCTCCAATTC | 10 |
| ARX | CTGCTGAAACGCAAACAGAG | 11 | CTCGGTCAAGTCCAGCCTCATG | 12 |
| | GC | | | |
| CRHR2 | GGCATCACCTACATGCTCTT | 13 | AAGAAACCCTGGAACGACTG | 14 |
| RBP4 | GCGATGGCAGATCAGAAAG | 15 | AGAGCTGAAGACTGAGAGCTA | 16 |
| | A | | | |
| Somatostatin | CCAACCAGACGGAGAATGAT | 17 | CCATAGCCGGGTTTGAGTTA | 18 |
| Insulin | CAGCCTTTGTGAACCAACAC | 19 | GGTCTTGGGTGTGTAGAAGAAG | 20 |
| Glucagon | TGCCAAACGTCACGATGAA | 21 | TCACCAGCCAAGCAATGAA | 22 |
| GAPDH | ATCACCATCTTCCAGGAGCG | 23 | TTCTCCATGGTGGTGAAGACG | 24 |
| | A | | | |

### Statistics

Error bars represent standard error of mean (SEM) as indicated in the figure legends. Prism v9.2.0 software was used to perform statistical analyses using ANOVA or a paired or unpaired student's t test, where appropriate. The qRT-PCR was performed using 3 independent biological samples as indicated in figure legends; each biological sample was run in triplicate except the single human cadaveric islet sample where it is run in three technical replicates (FIG. 6B).

### Example 3: Development of a Delta Cell Progenitor (DP) Population

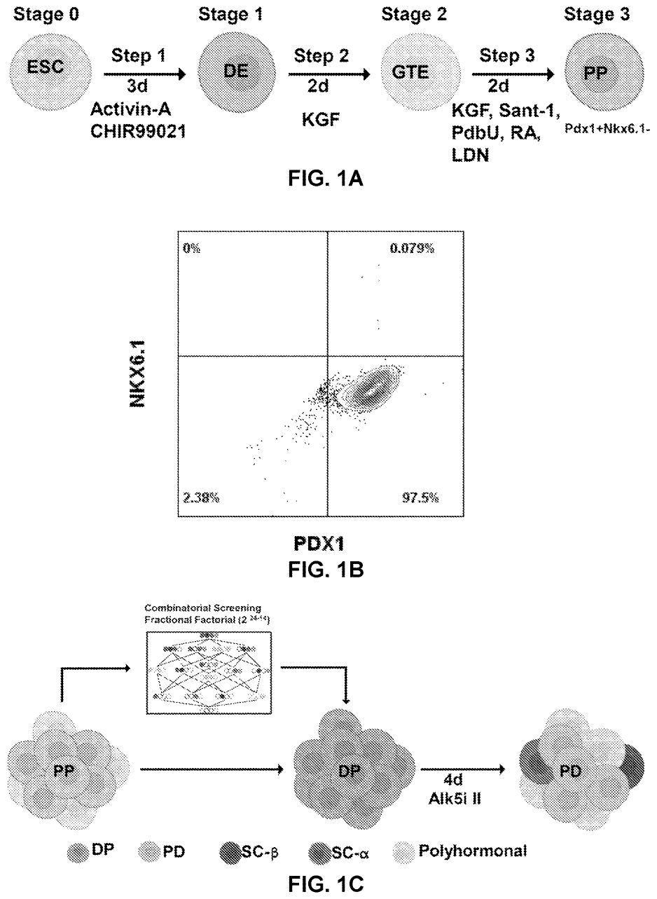

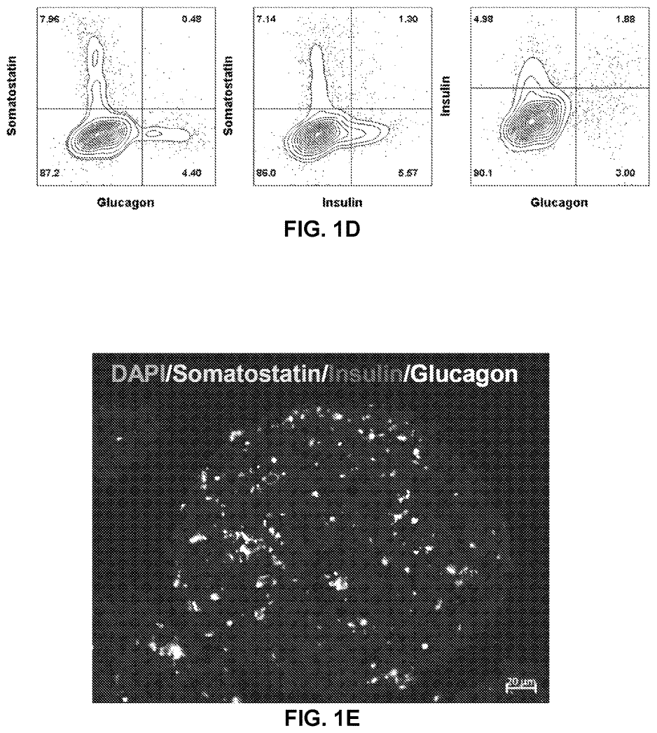

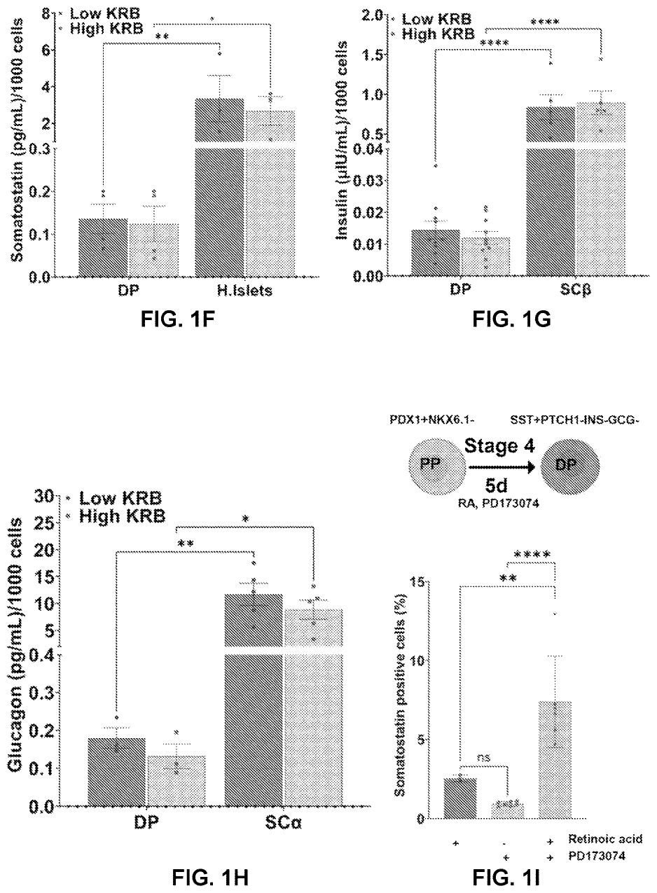

Protocols for the generation of SC-α and SC-β cells may result in a small number of somatostatin positive cells, suggesting that a minor cell population in differentiation protocols may be capable of differentiating toward a delta lineage. While protocols for the differentiation of human PSC toward alpha and beta cells have been developed, to date protocols toward pancreatic delta cells have been elusive. Toward this end, conditions were identified that could promote commitment to a delta cell lineage (FIG. 1A). Induction of the transcription factor NKX6.1 in PDX1 positive pancreatic progenitors is one step involved in early and late pancreatic beta cell specification. Furthermore, suppression of NKX6.1 in adult mouse beta cells activated NGN3 expression in beta cells and converted them to somatostatin expressing delta cells. As such, a PDX1⁺/NKX6.1⁻ (PDX1 positive/NKX6.1 negative) pancreatic progenitor could be a useful starting population for efforts to differentiate toward delta cells.

Modulation of the differentiation conditions in early stages of differentiation of beta cells could be used to promote a PDX1⁺/NKX6.1⁻ progenitor population (see, e.g., Kelly et al., Nat. Biotechnol., 29:750-756 (2011)). Adapting this modification to the differentiation schema generated a population of cells that was 97.58±10.71 percent Pdx1 positive. Of note, less than 1 percent of these cells express Nkx6.1 (PDX1⁺/NKX6.1⁻) (FIG. 1B and FIG. 13A).

While there are defined markers of mature delta cells, there is considerably less known about the signals that promote delta cell commitment and the molecular markers of delta cell progenitors. To identify signals that promote the development of a delta cell progenitor, a combinatorial small molecule screening approach was used, which allowed for reduced bias in pathway identification involved in delta specification. PDX1⁺/NKX6.1⁻ PP cells were dissociated and plated in 384-well multiwell plates, where they were then introduced to a defined set of chemical combinations for 4 days followed by endocrine-inducing compound Alk5inh II (ALK5 inhibitor II, Alk5i, CAS No. 446859-33-2, 2-[5-(6-methylpyridin-2-yl)-1H-pyrazol-4-yl]-1,5-naphthyridine). This screening format allowed for identification of conditions that could promote a population committed to a delta cell lineage upon endocrine induction.

The combinatorial screening approach used herein was designed to evaluate the interactions of several small molecules in concert to identify signaling pathways involved in a differentiation process. This approach was termed “PIECCS” (Pathway and Interaction Elucidation through Combinatorial Chemical Screening). In brief, this approach uses Design of Experiment (DOE) and fractional factorial statistical method to dramatically reduce the set of all possible combinations to a smaller test set that can provide inferences on the most involved pathways interactions.

In this instance, the resulting cell populations were stained with somatostatin after endocrine induction to determine the combinations that promoted a progenitor population that was capable of differentiating to somatostatin secreting cells. Counter staining with glucagon and insulin allowed for the elimination of combinations that produced polyhormonal cell populations. The wells in which the percentage of somatostatin positive but insulin and glucagon negative cells was higher than that in control DMSO were considered as primary hits.

This screening method identified a combination of two small molecules: PD173074 (CAS No. 219580-11-7, 1-tert-butyl-3-[2-[4-(diethylamino)butylamino]-6-(3,5-dimethoxyphenyl)pyrido[2,3-d]pyrimidin-7-yl]urea) and Retinoic Acid (RA). Treatment with these molecules generated 4.15±0.41 percent of delta progenitor capable of differentiating into SCδ cells, 7.38±1.19 percent of total somatostatin positive cells, 13.01±1.16 percent of total insulin positive cells, and 2.83±0.44 percent of total glucagon positive cells (FIG. 1D-E, FIG. 5B, FIG. 8A, FIG. 10B, and FIG. 14). However, somatostatin protein was not detected in western blot at this stage (FIG. 6C and FIG. 12A-B).

### Example 4: Inhibition of FGF Receptors in Presence of RA is Required for Differentiation to Delta Progenitors (DP)

While retinoic acid plays a role in pancreatic development, much less is known regarding the role of FGF receptor biology in the establishment of delta cells. To determine the relative contributions of retinoic acid and PD173074 in the induction of a delta cell progenitor population, the ability of these compounds to induce somatostatin expression independently was investigated. Stage 3 cells were treated with either retinoic acid, PD173074, or a combination of these compounds for 5 days (FIG. 11). Although both RA and PD173074 induced a small population of somatostatin expression in PP cells when treated independently, an increased expression of somatostatin was observed when treated in combination for a period of 5 days (FIG. 1I). Therefore, the compound PD173074 works synergistically with RA to efficiently induce somatostatin expression and generate delta progenitor cells (FIG. 1C).

To further explore the specificity of inhibition of FGF receptors, PP cells were treated with five additional FGF receptor inhibitors (AZD4547, erdafitinib, roblitinib, pemigatinib, or BGJ398) along with RA (FIG. 7). Although all chosen compounds inhibit FGF signaling, these inhibitors have different receptor specificities. To find an effective concentration of these compounds, differentiated PP cells were transferred to six-well plates and treated with either control having only RA or each FGF receptor inhibitors along with RA for 5 days and quantified the percentage of somatostatin expressing cells. RA alone in the control group resulted in only 2.53±0.15 percent of somatostatin expressing cells, and all the FGF receptor inhibitors tested showed dose dependent increase in somatostatin expressing cells with an effective concentration of 200 nM and that was significant when compared to that in the control group (FIG. 7C). Further, a similar experiment was repeated in small spinner flasks with an effective dose of 200 nM, and all FGF receptor inhibitors significantly increased the percentage of somatostatin positive cells compare to control group (FIG. 7D). These results demonstrate that inhibition of FGF signaling, while activation of RA signal efficiently, could induce a somatostatin permissible environment in PP cells (FIG. 1C).

### Example 5: Generation of Pre-Delta (PD) Cells

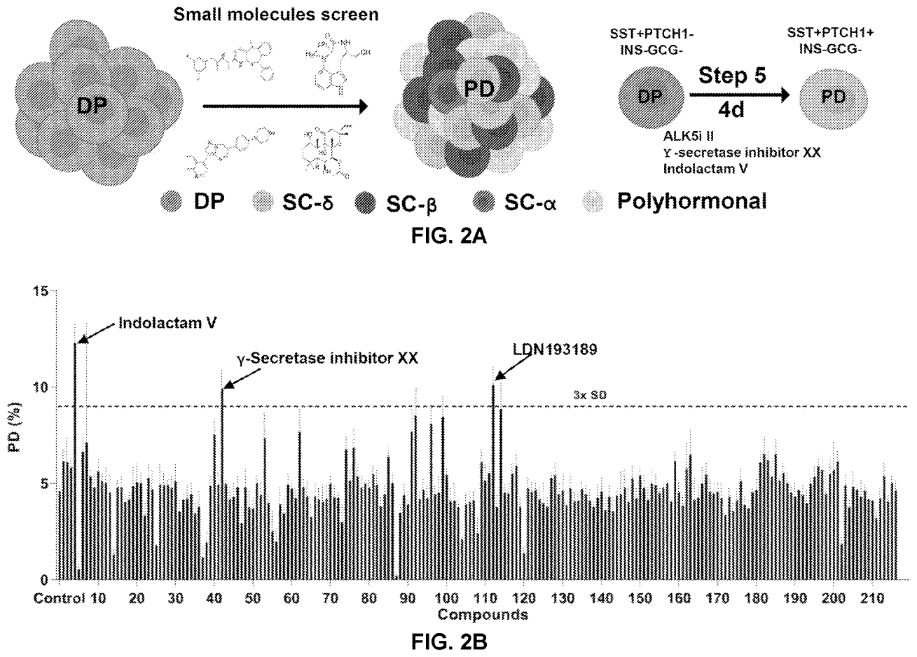

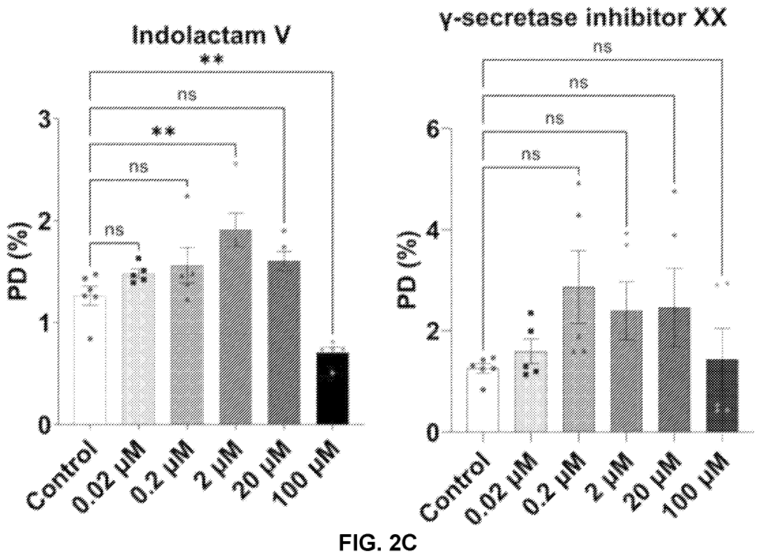

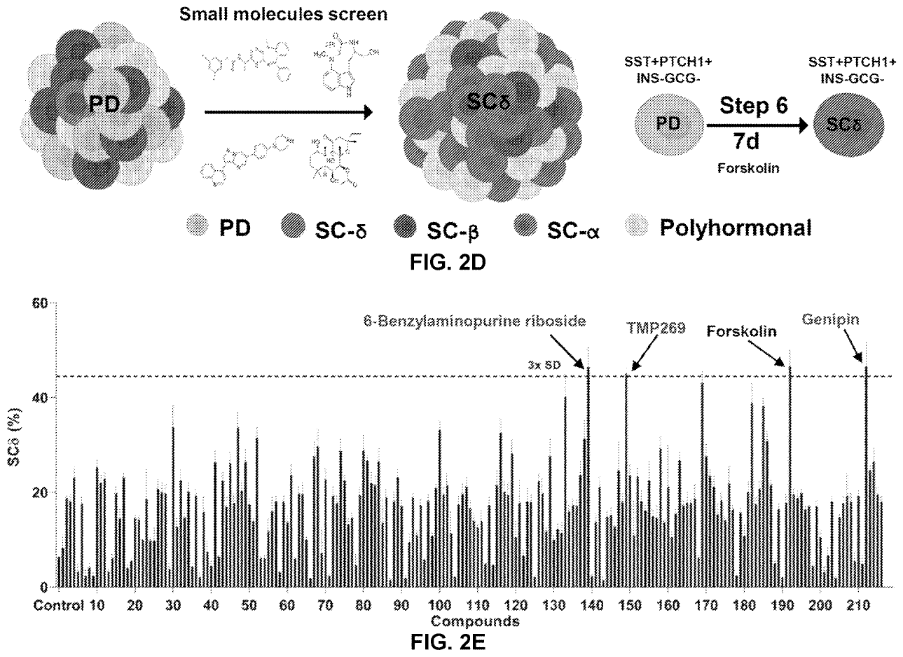

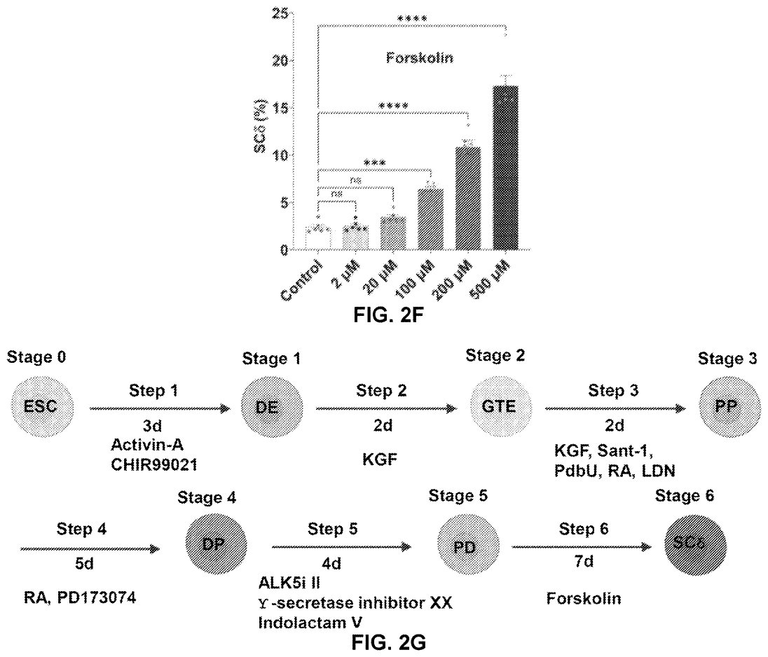

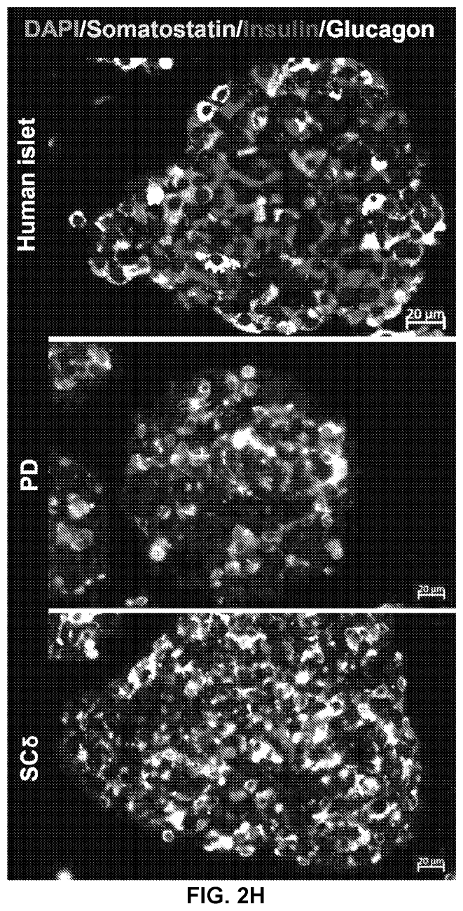

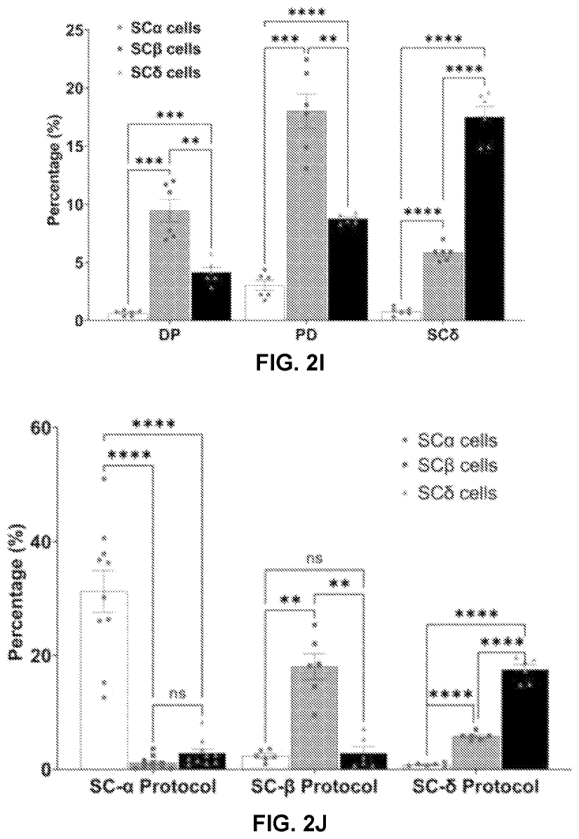

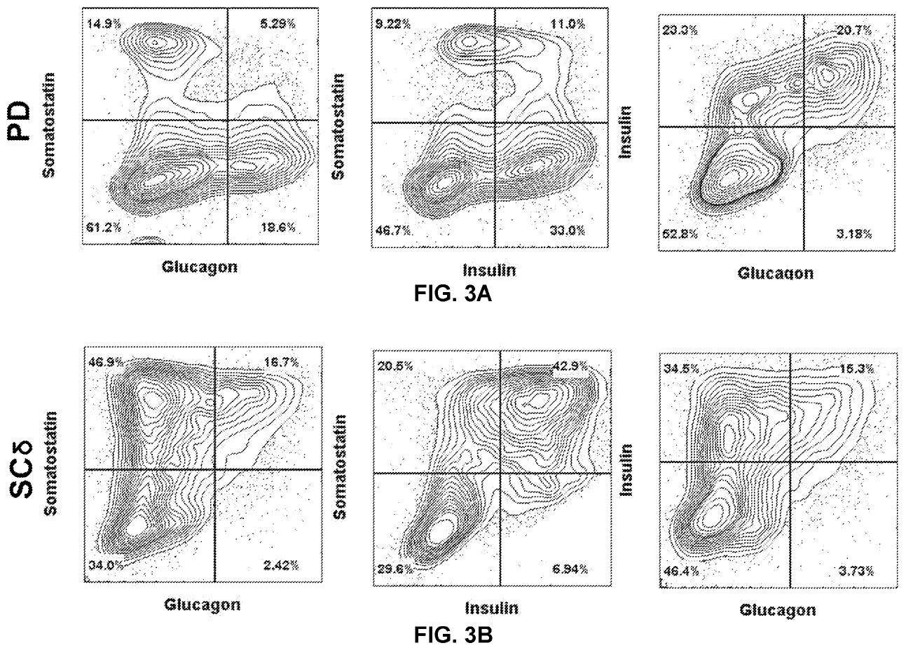

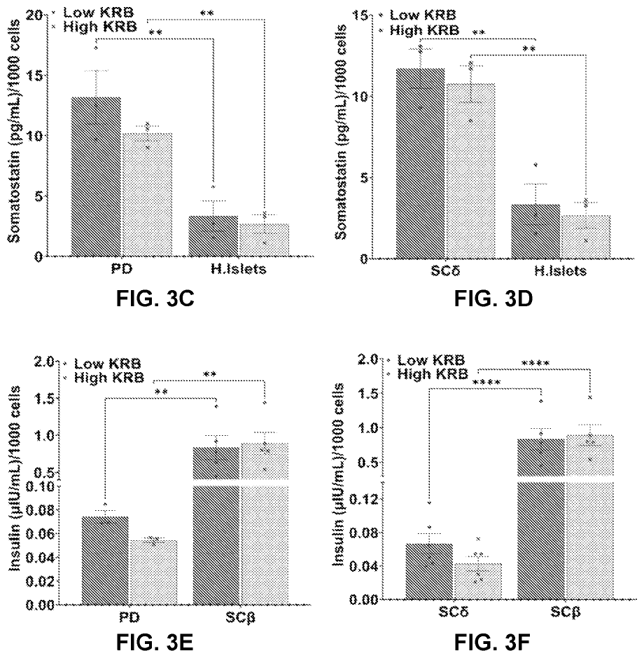

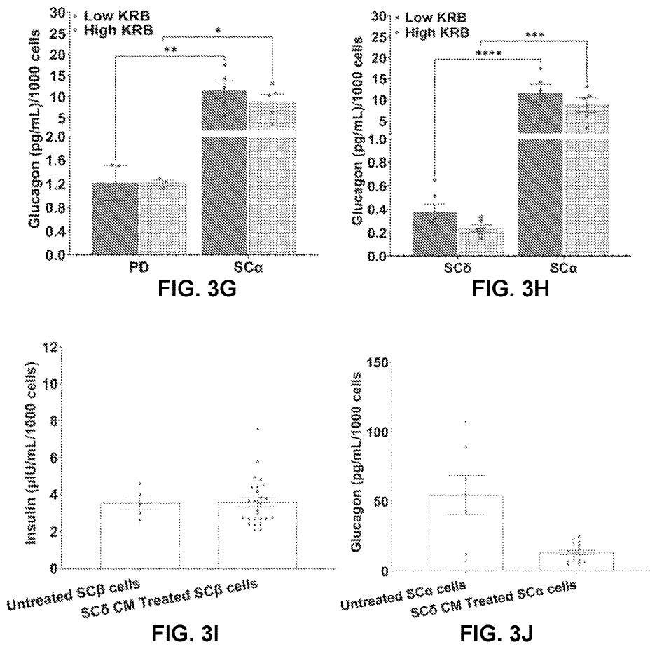

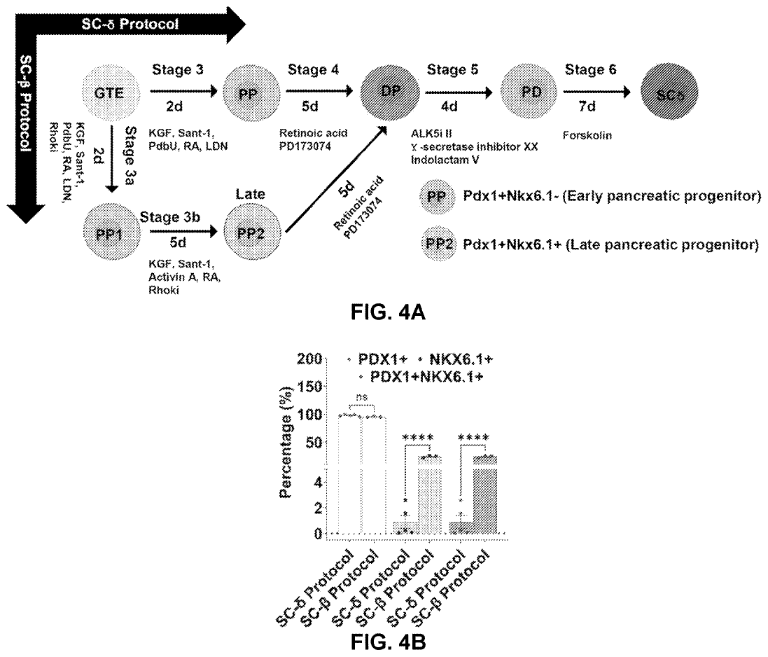

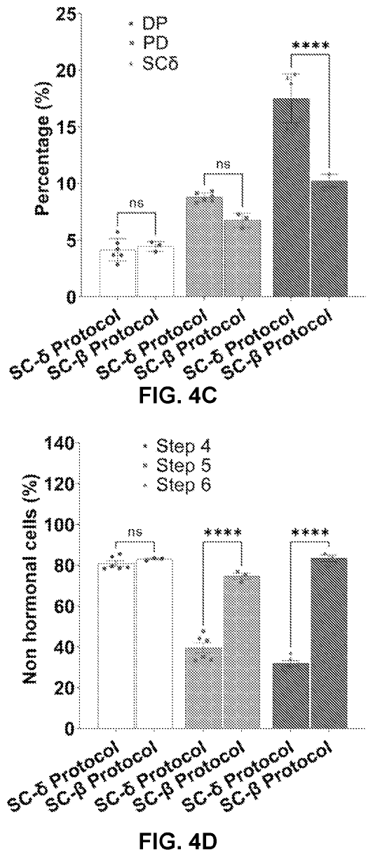

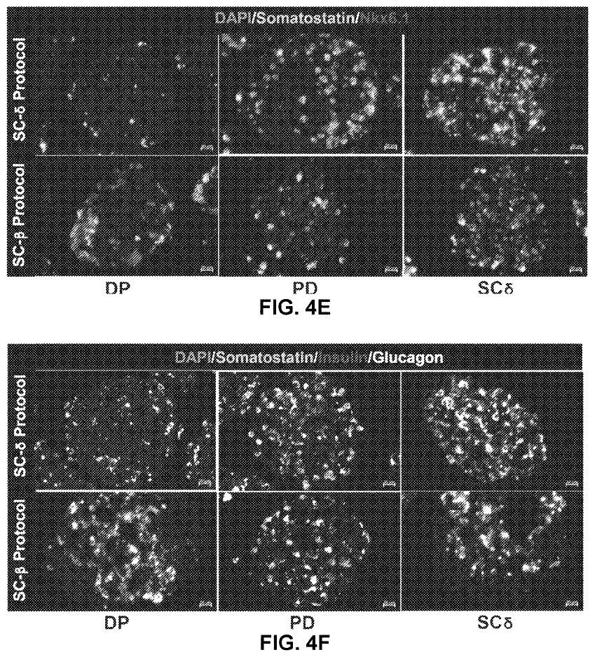

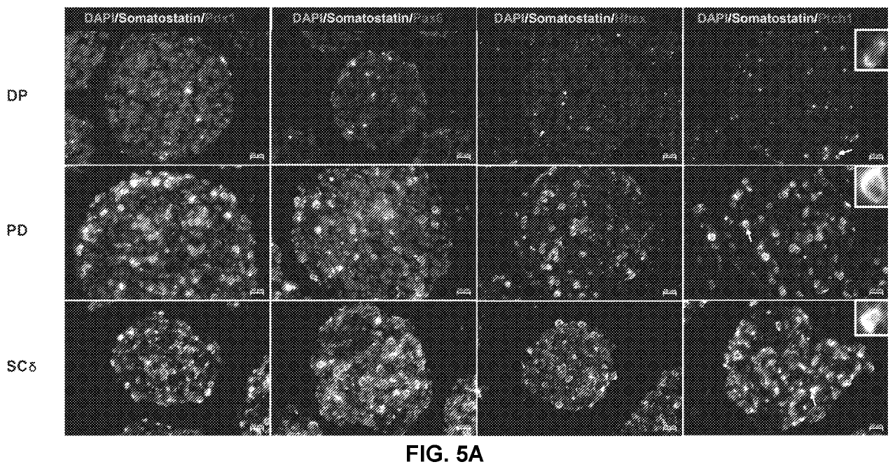

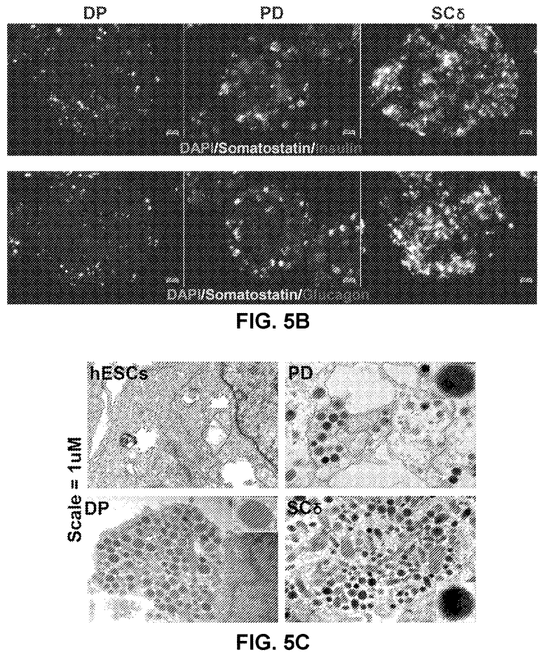

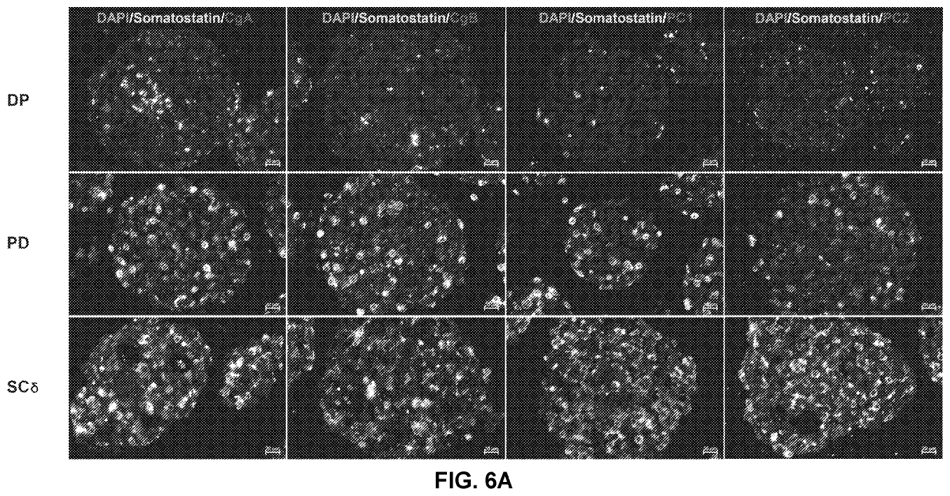

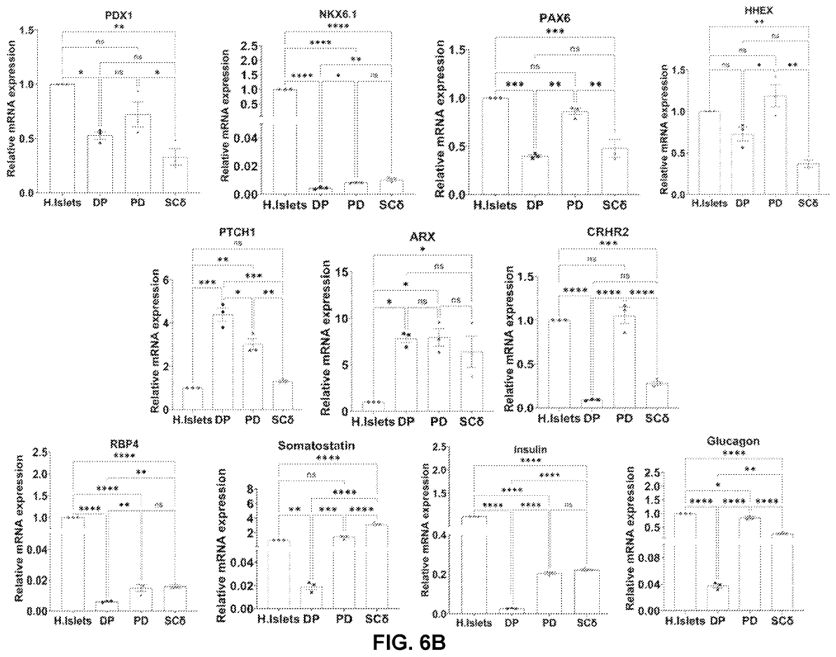

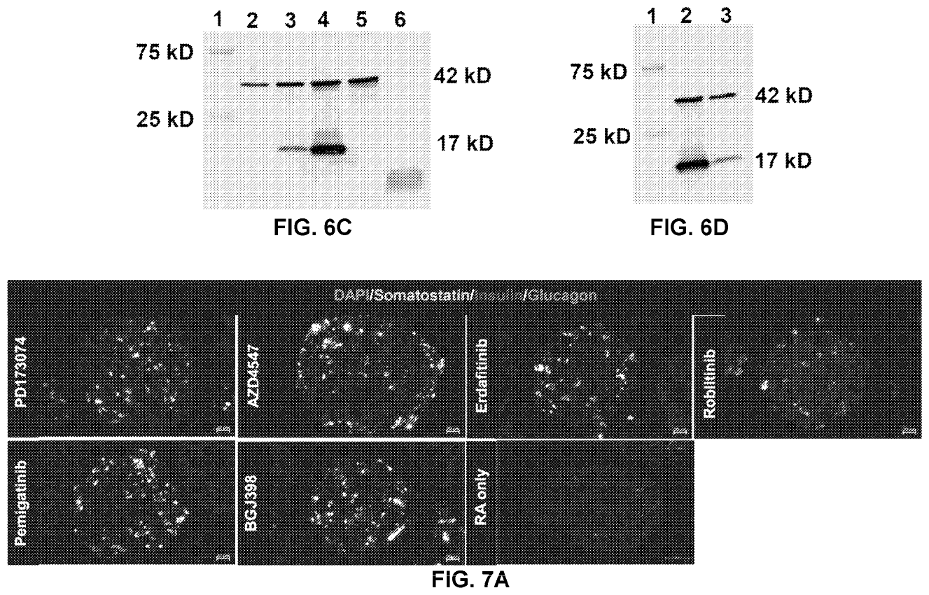

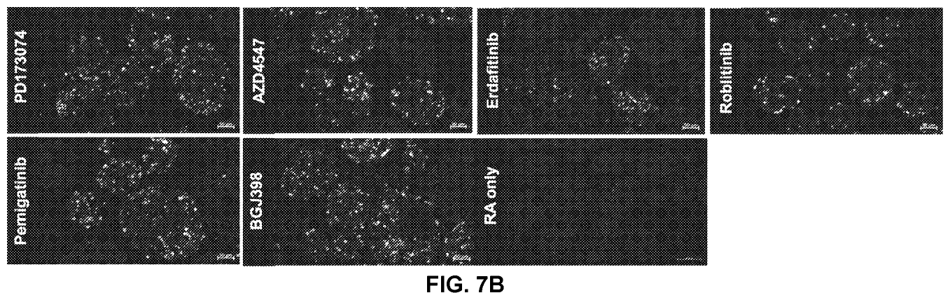

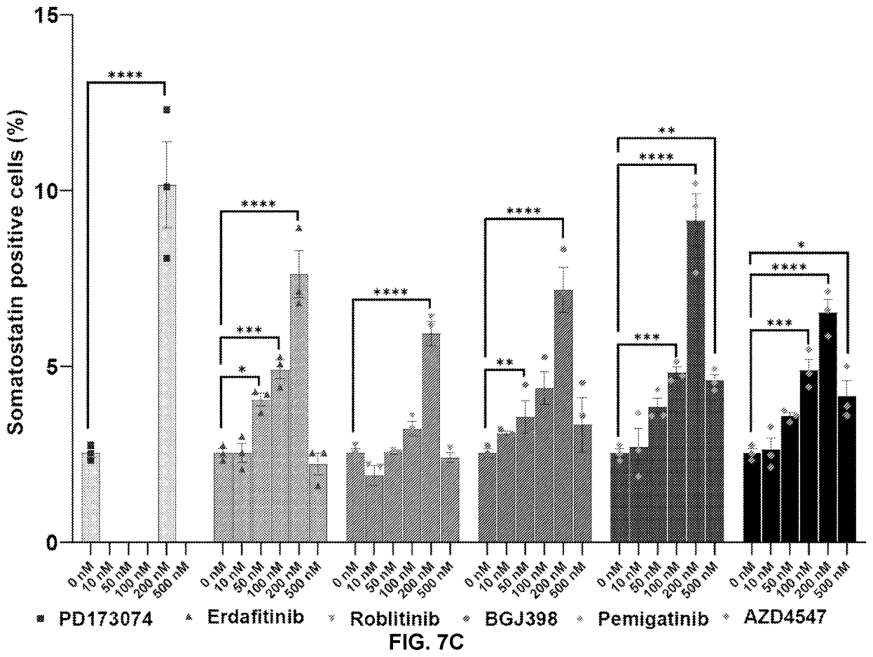

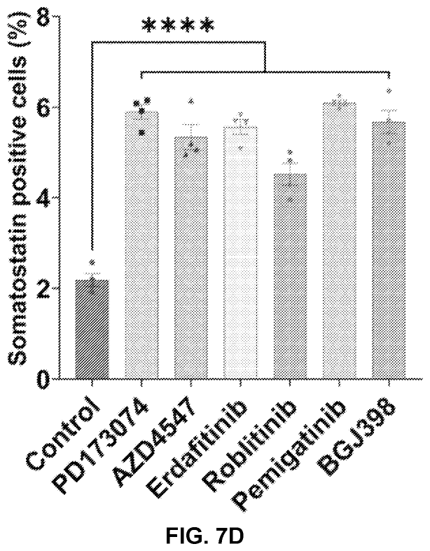

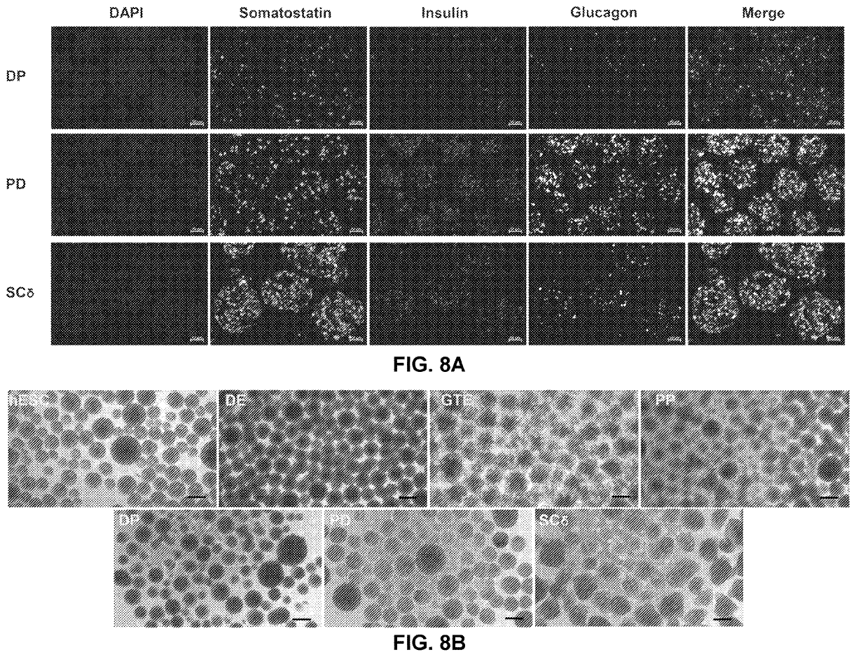

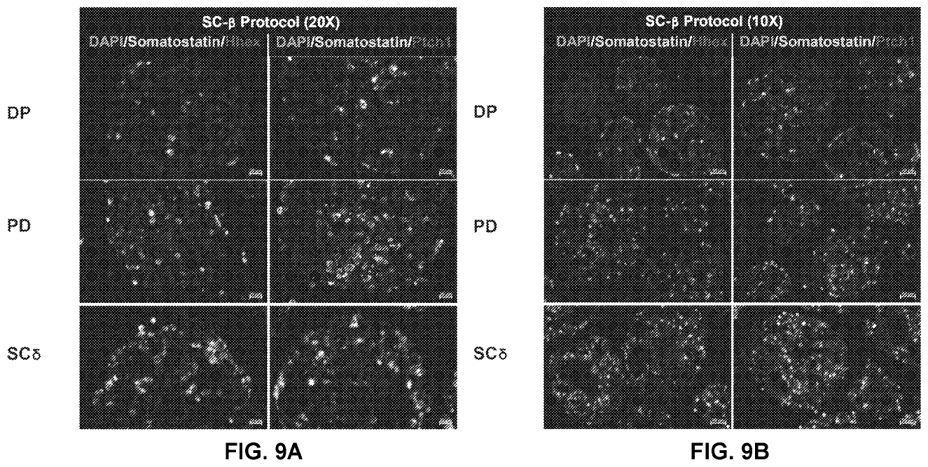

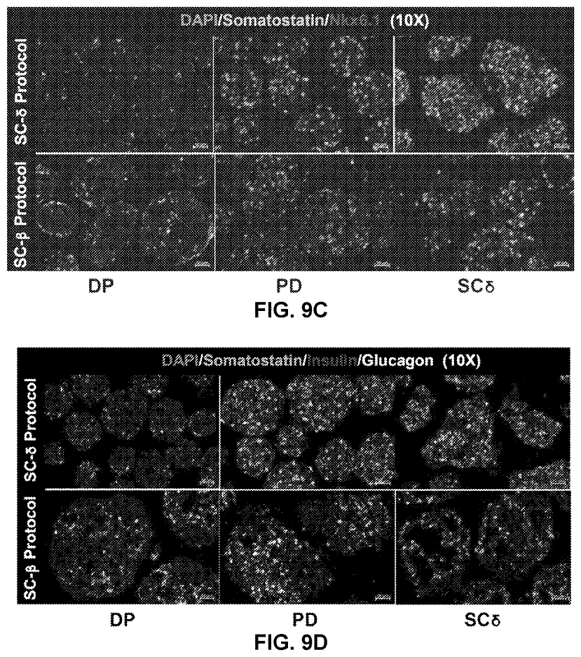

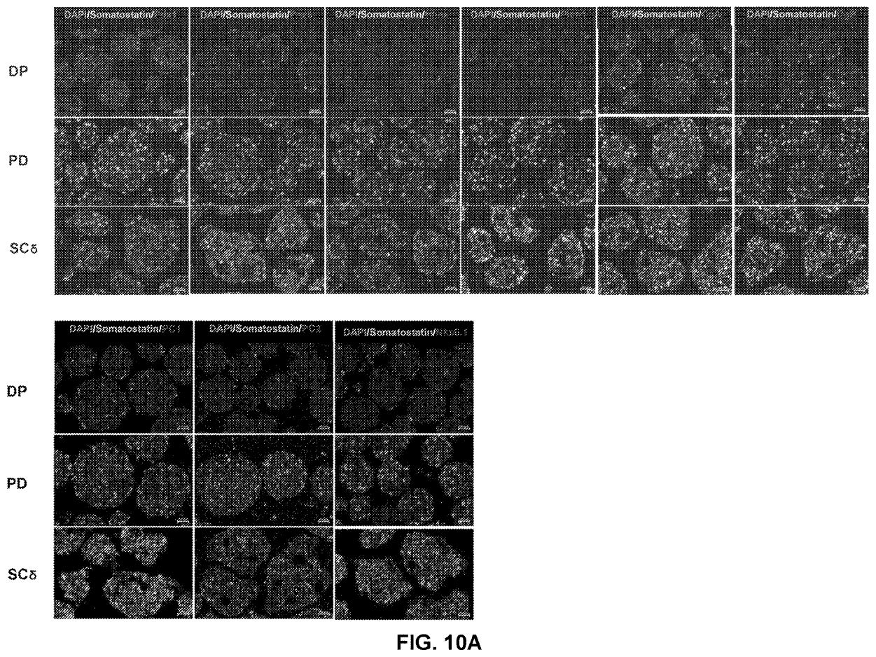

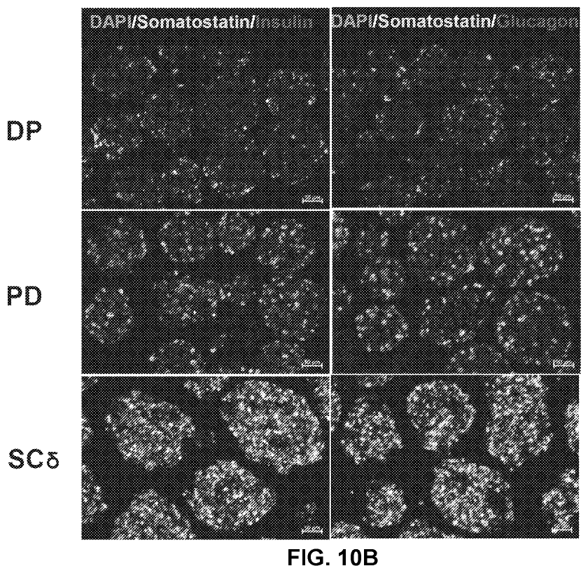

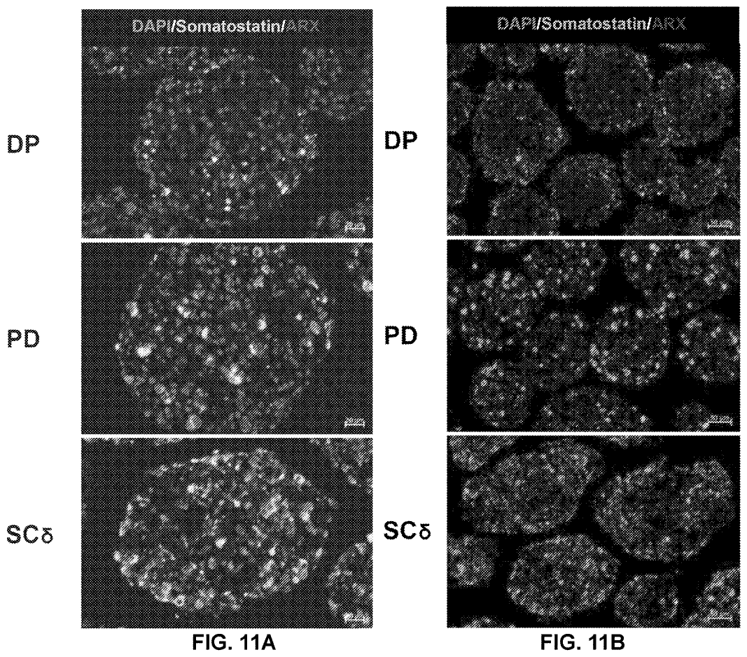

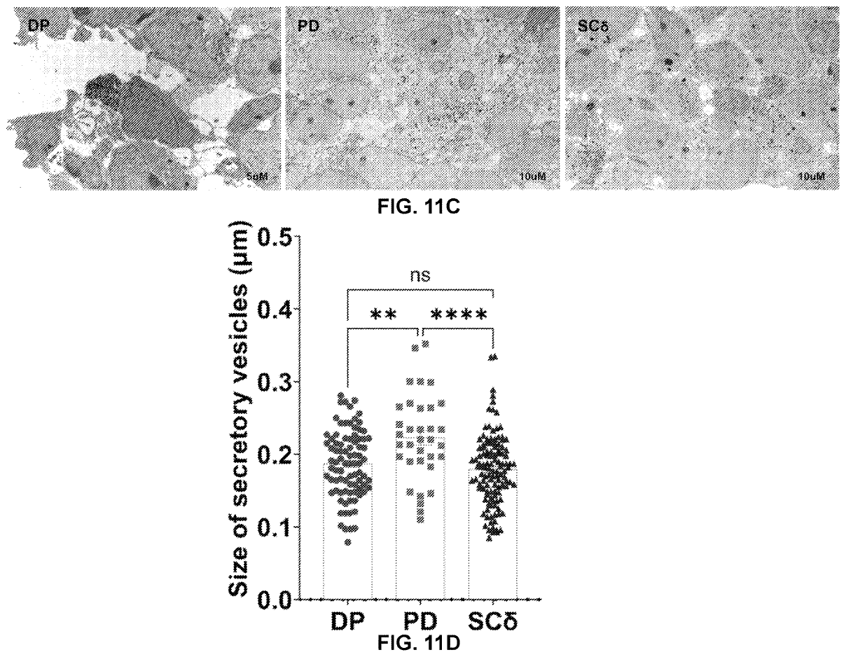

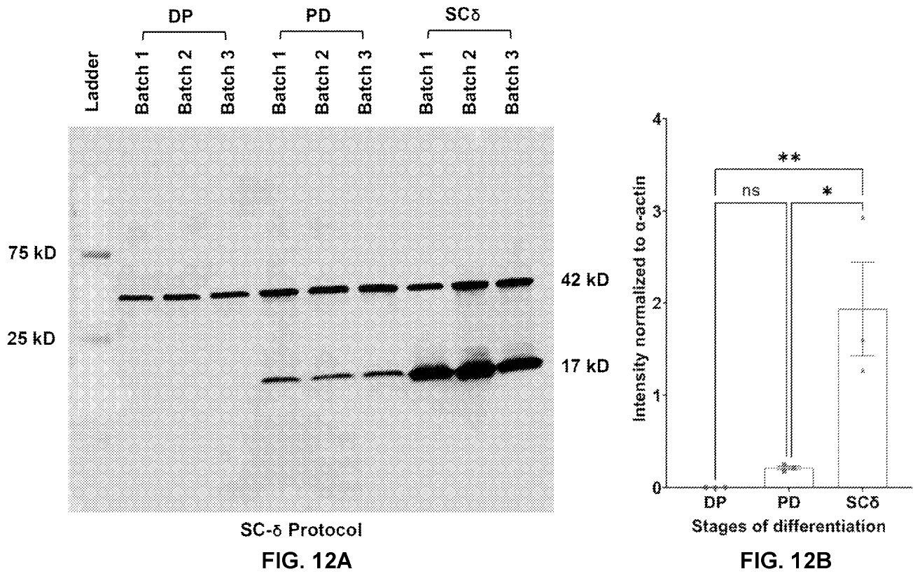

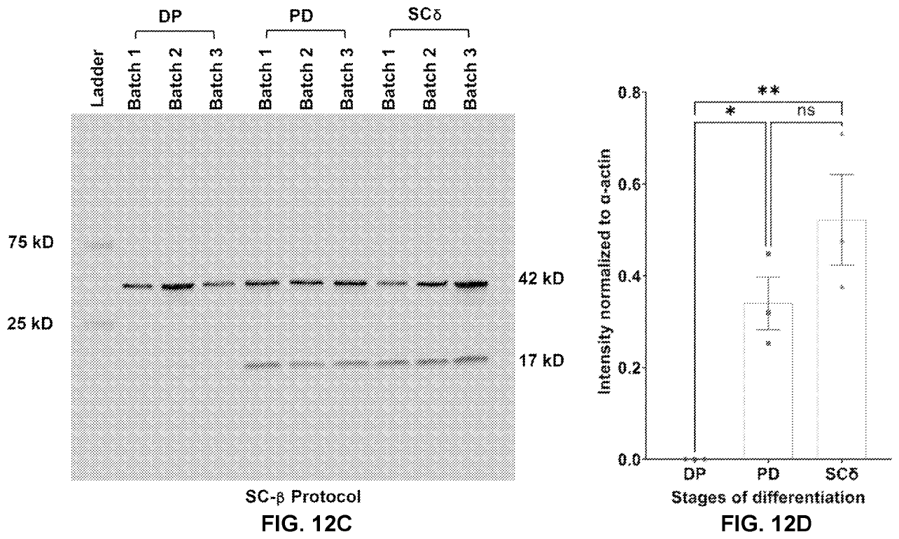

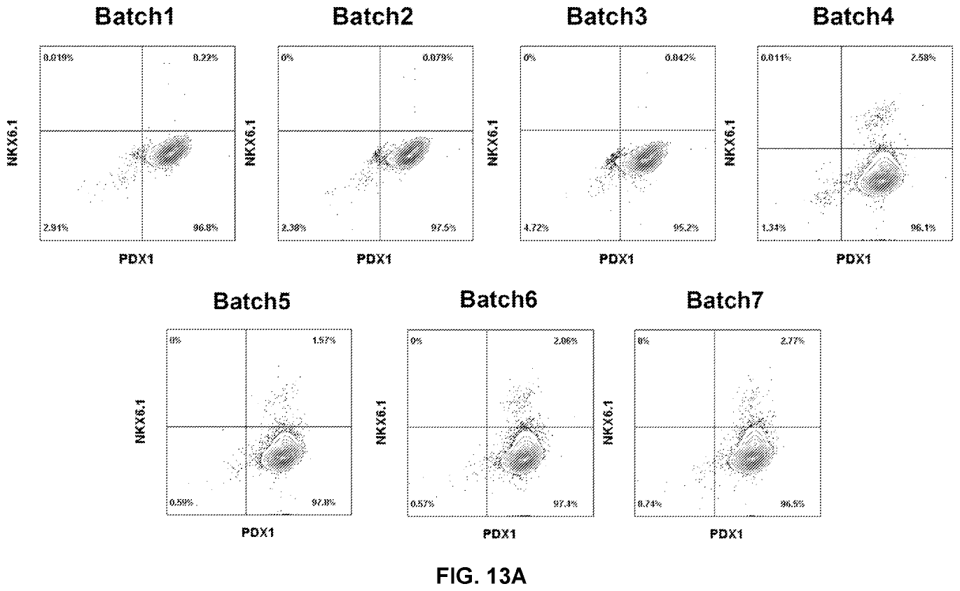

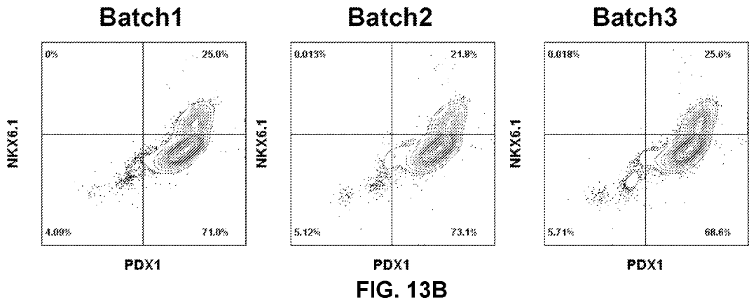

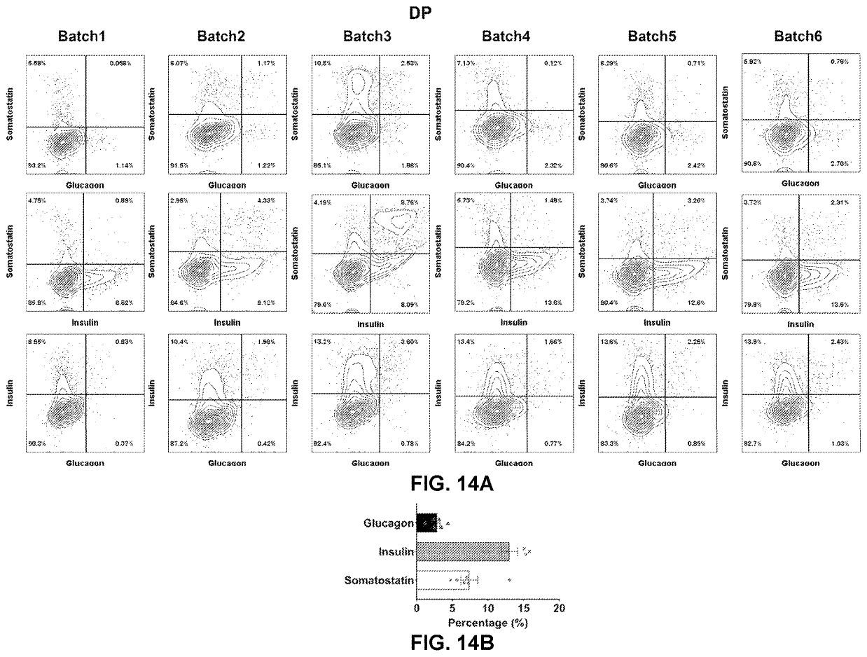

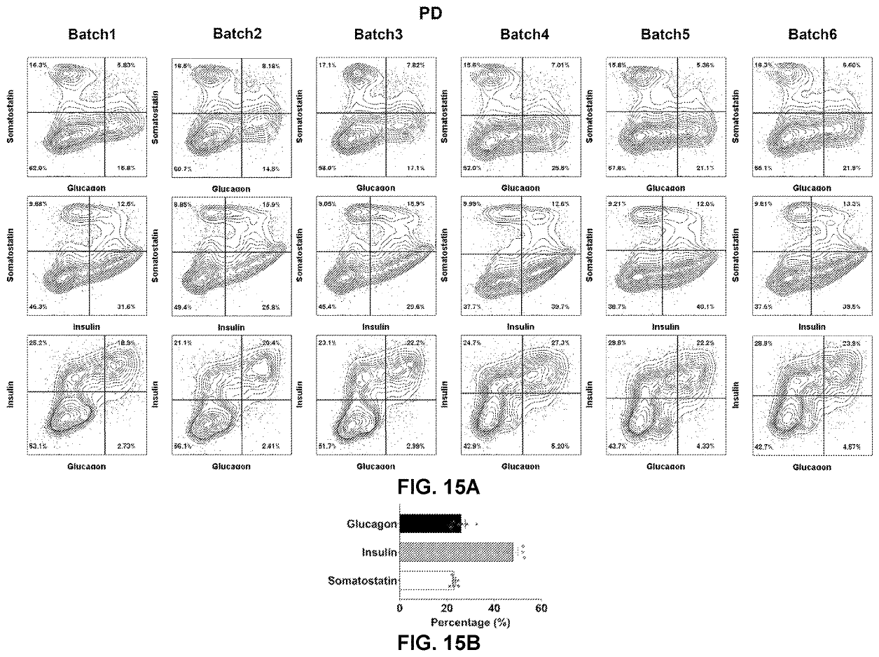

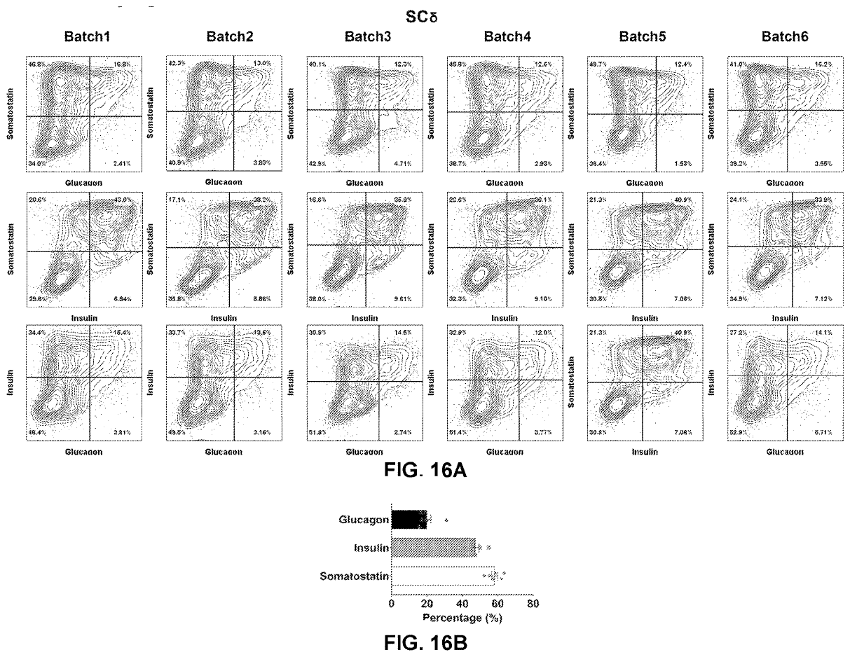

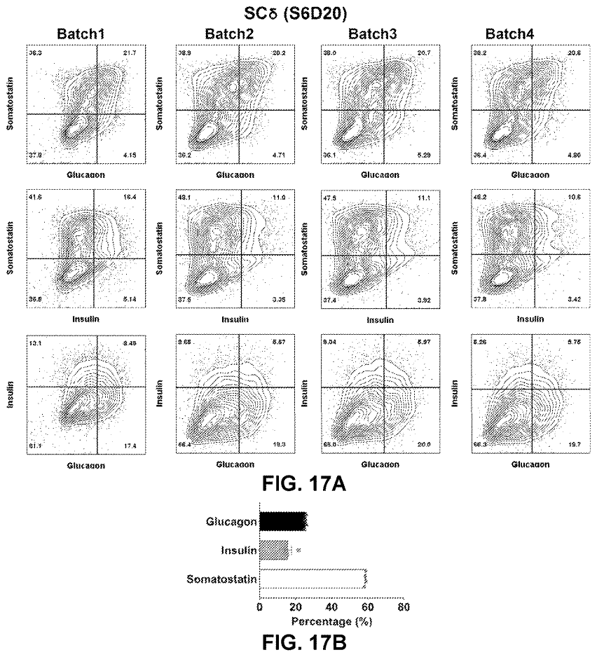

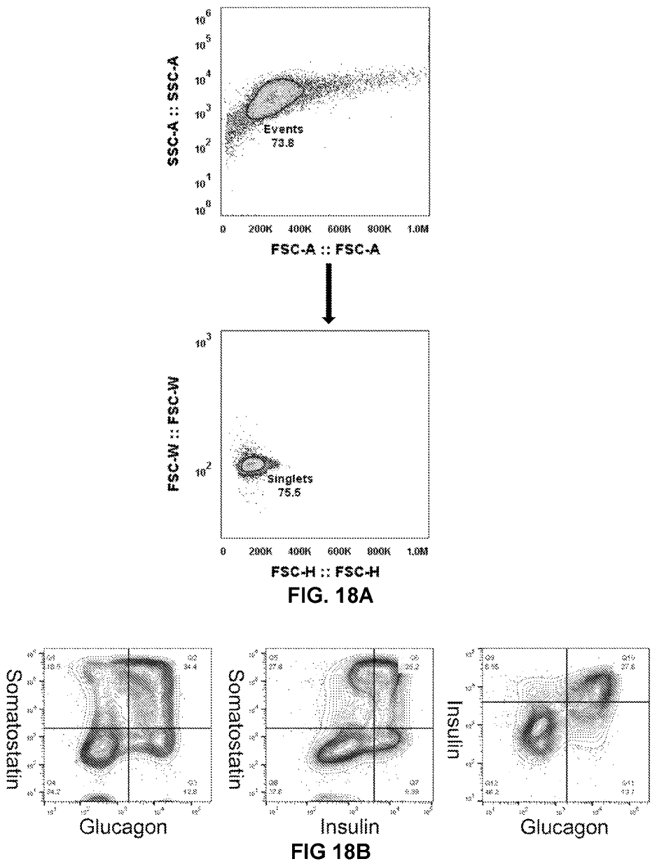

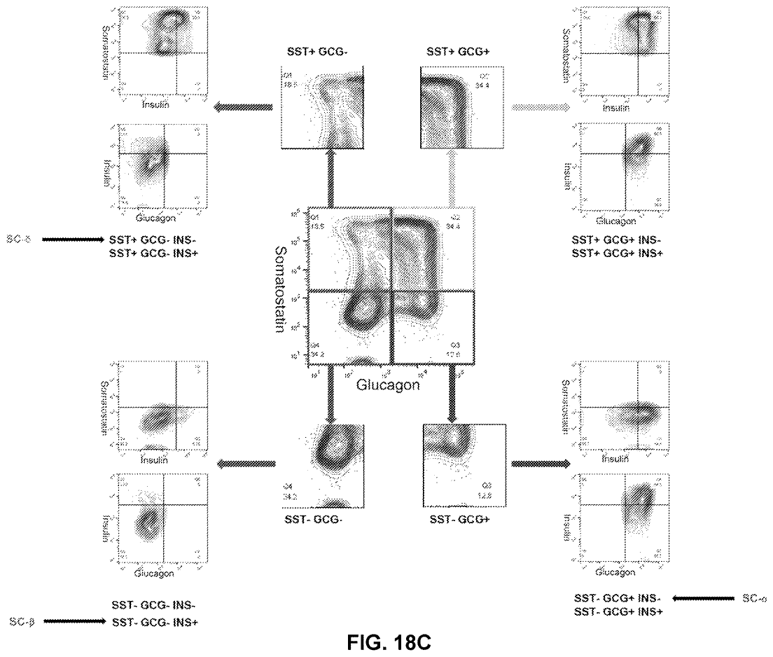

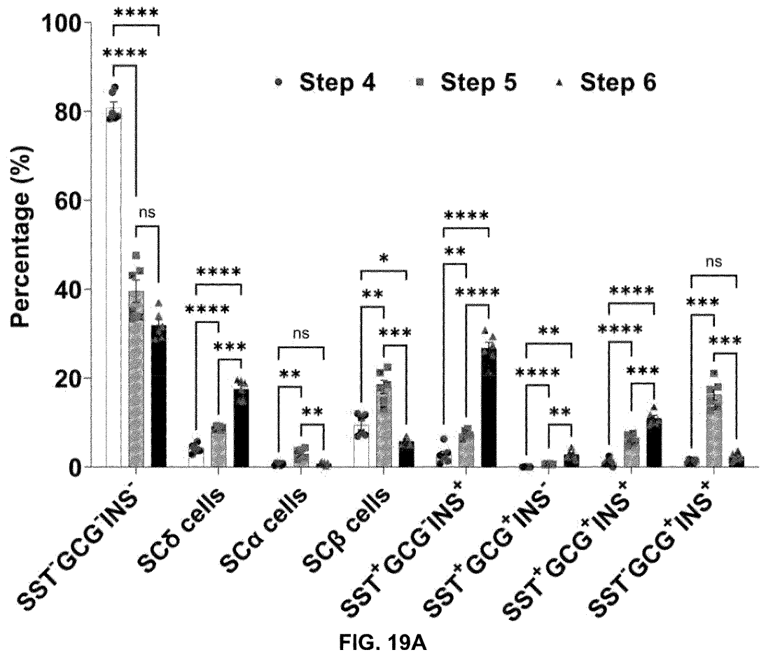

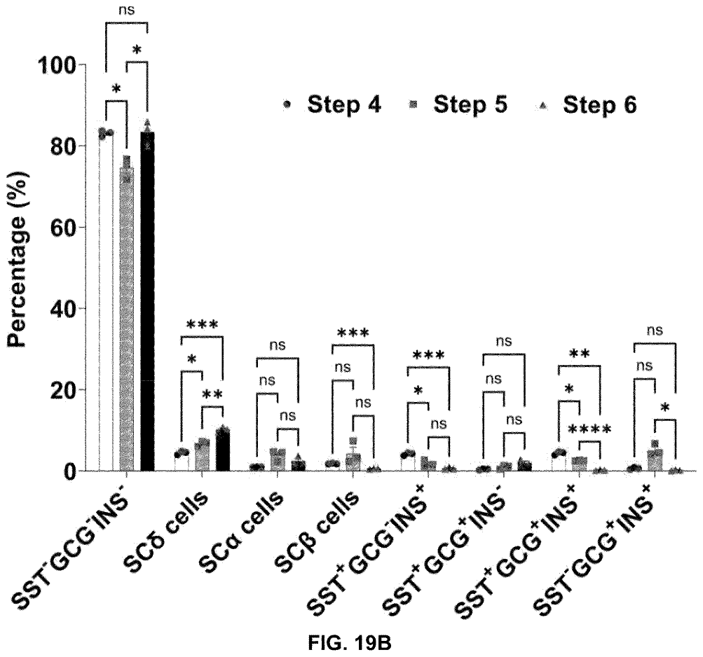

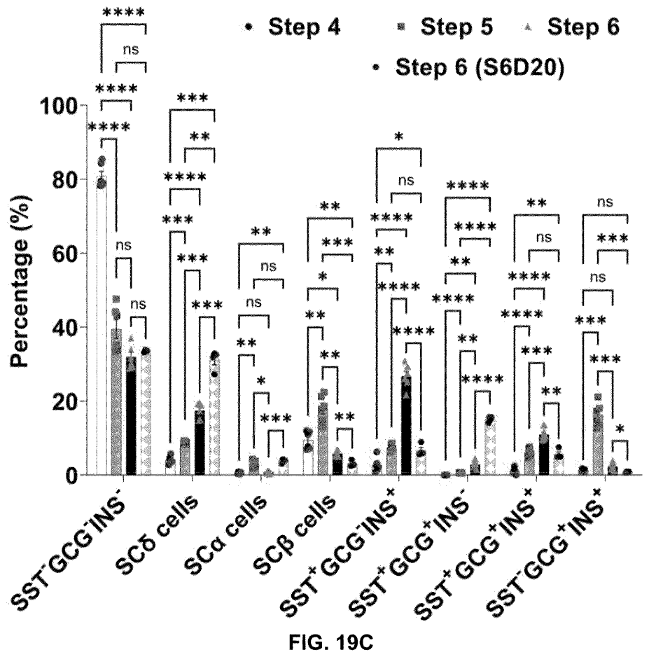

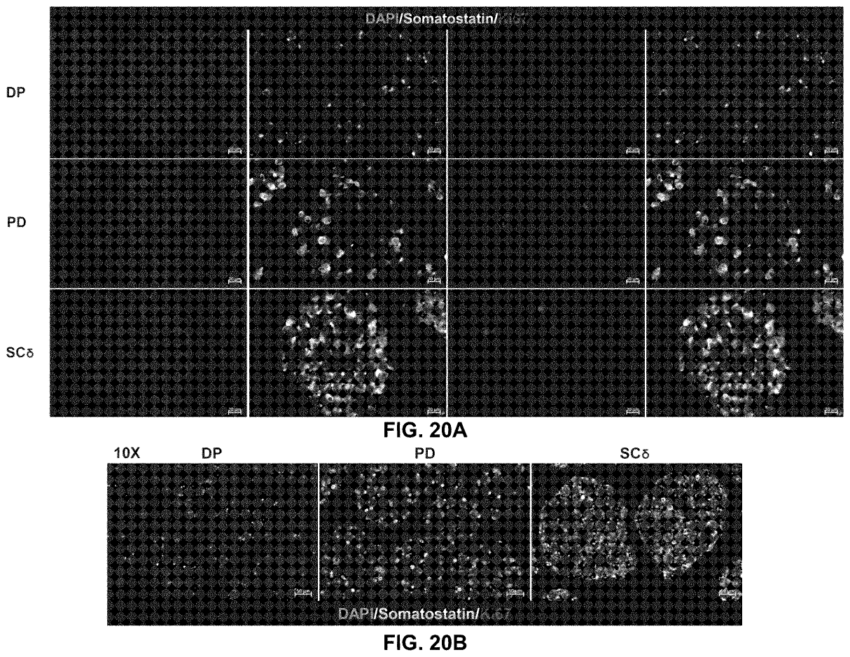

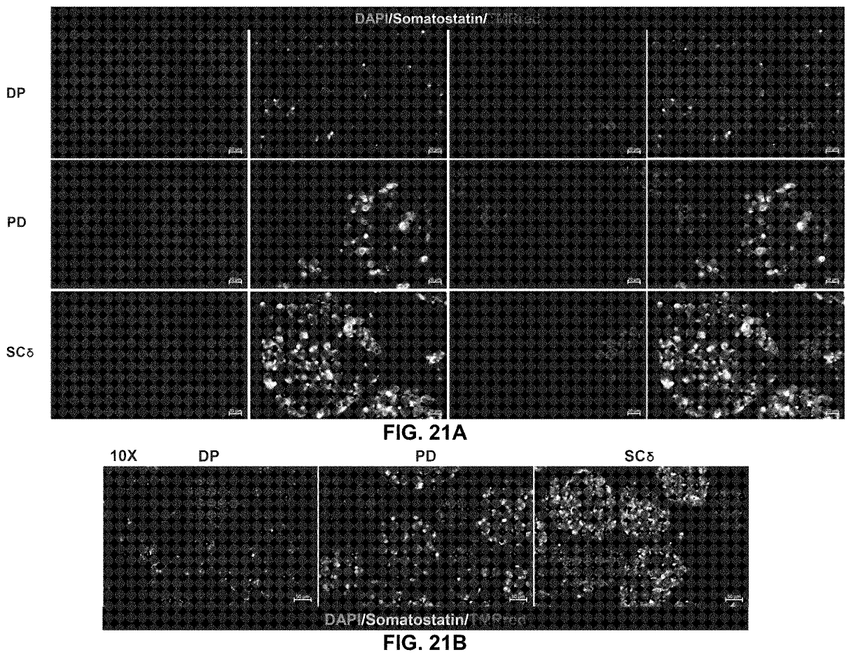

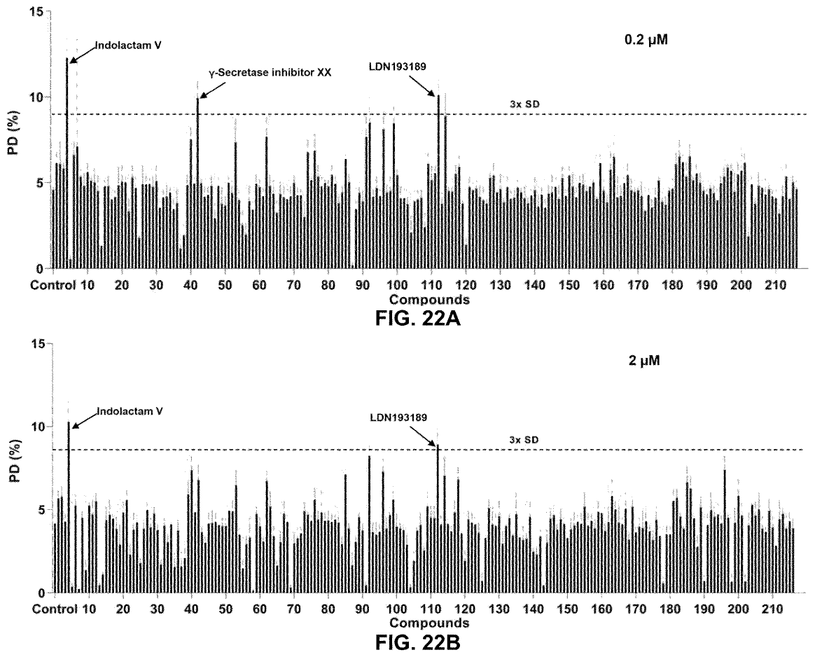

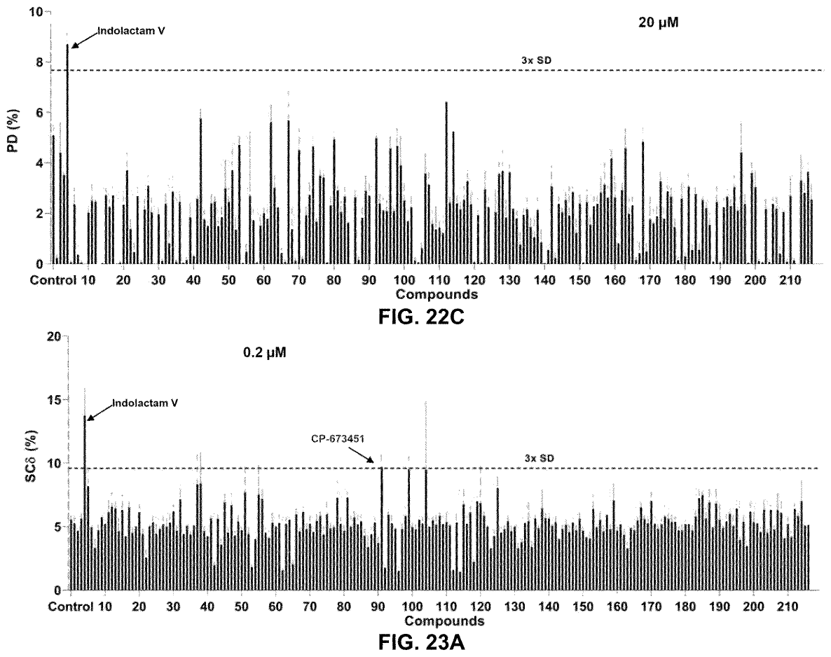

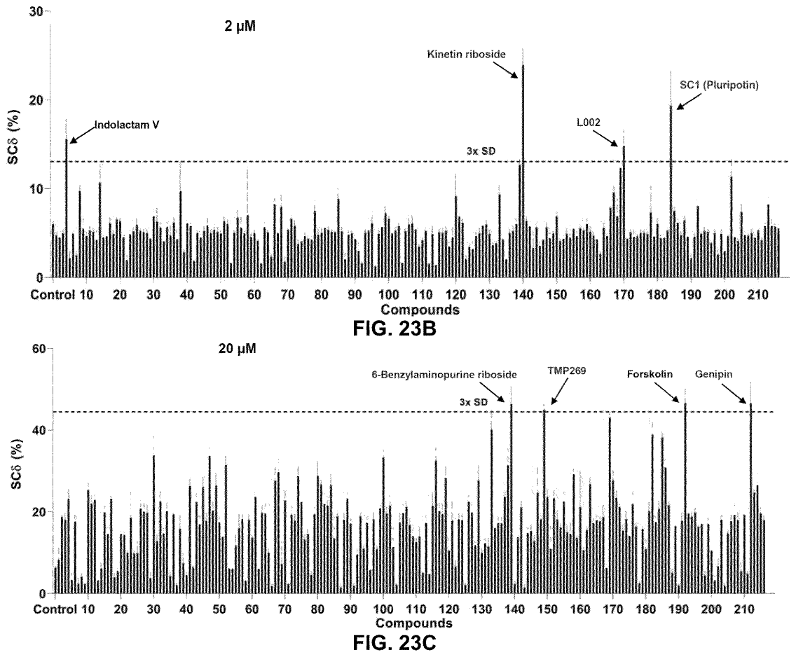

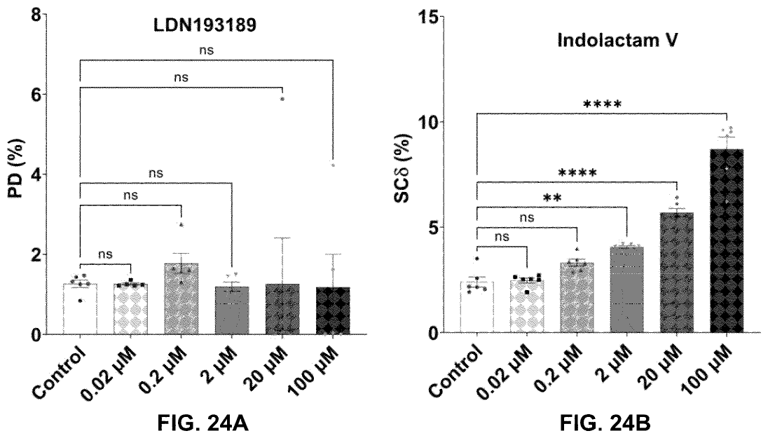

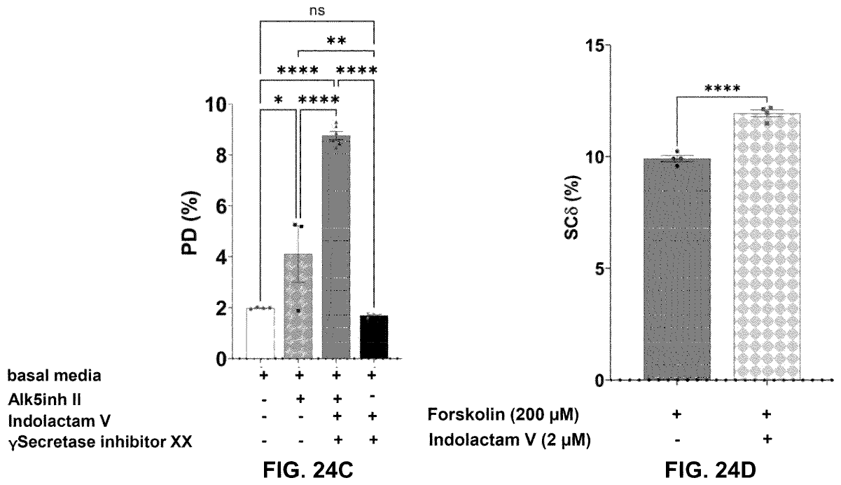

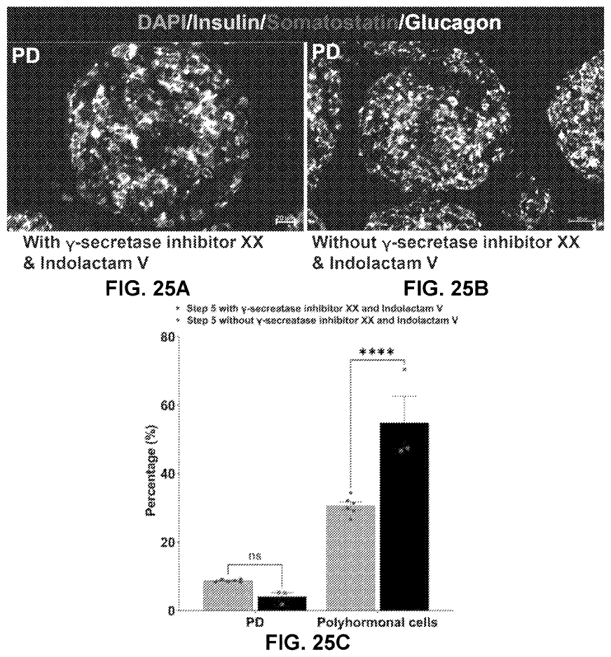

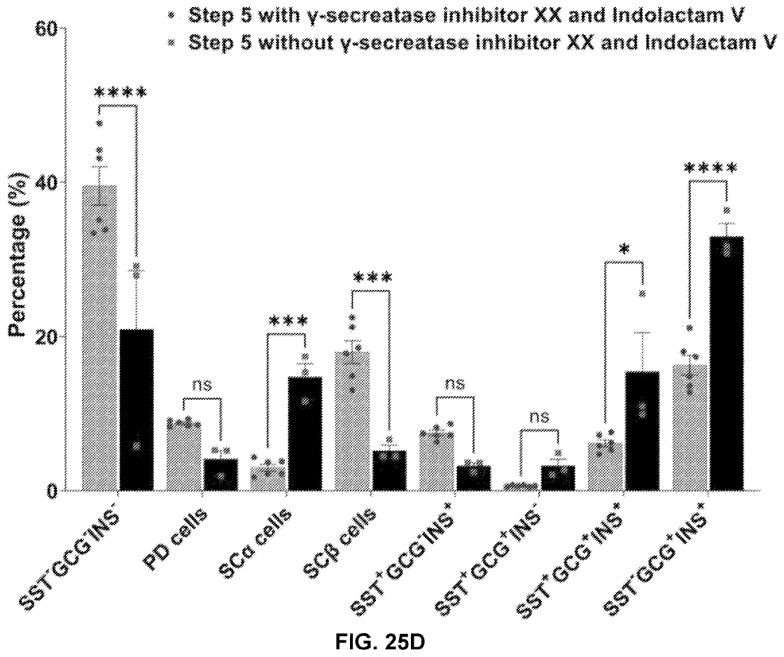

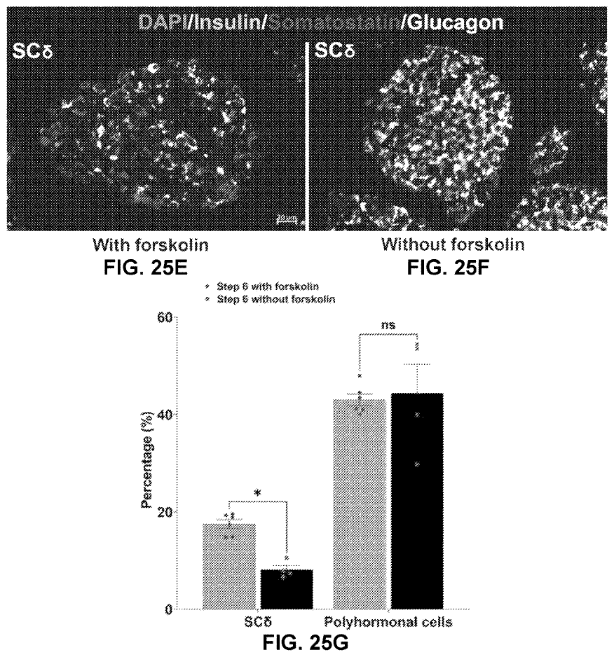

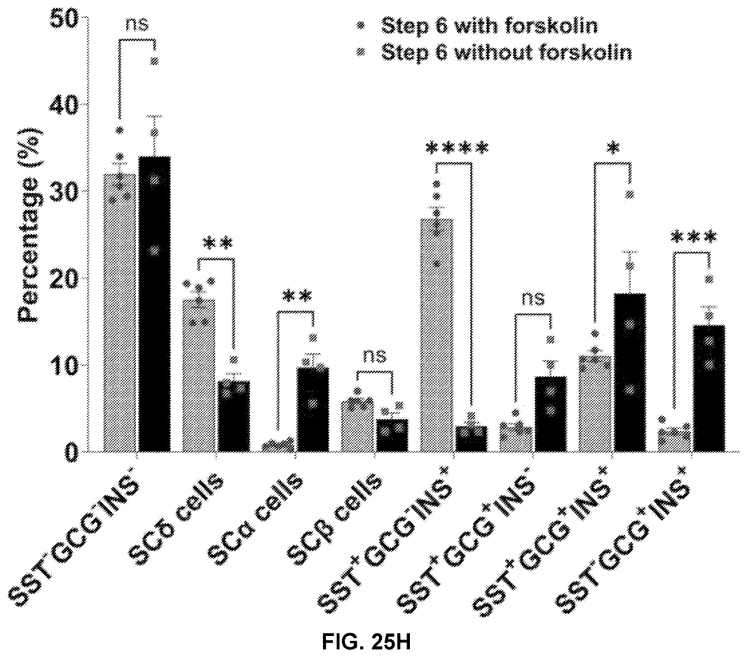

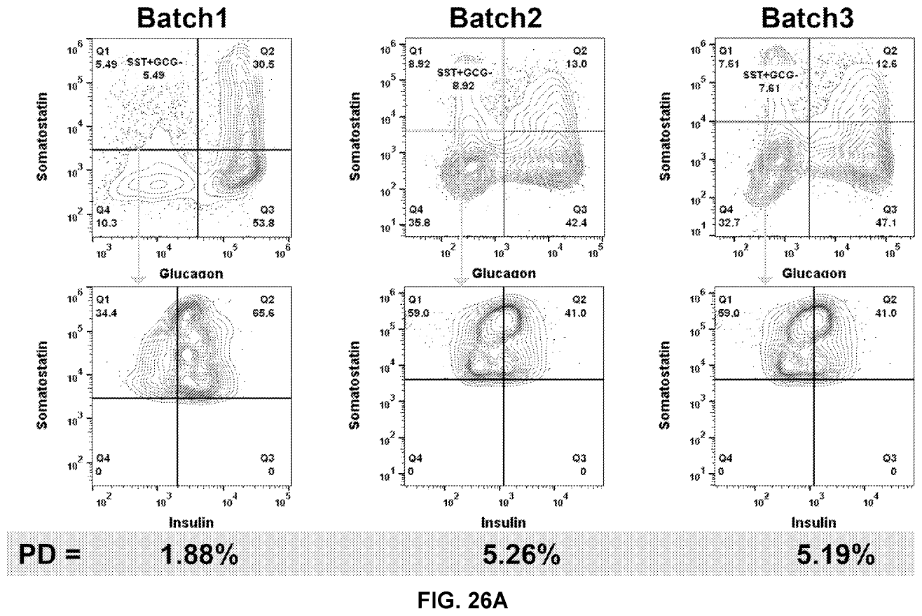

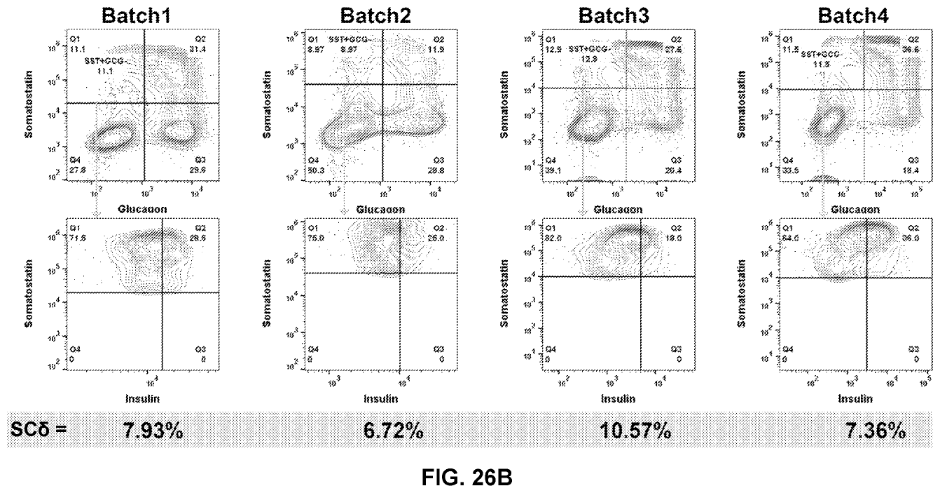

Inhibition of TGFβ signal with Alk5 inhibitor II improved endocrine differentiation. delta progenitor cells that were treated with Alk5 inhibitor II resulted in generation of 4.11±1.12 percent SC-delta cells (FIGS. 23C, 25C, and 26A). However, generation of a large number of polyhormonal cells was observed (FIG. 25B-C). Therefore, to mature the delta progenitor cells into a delta cell lineage, a small molecule screen was performed on delta progenitor cells by using an in-house library (FIG. 2A). Compounds that increased the percentage of Pre-Delta (PD) cells (positive for somatostatin (SST) while negative for both insulin (INS) and glucagon (GCG)) and reduced the percentage of polyhormonal cells were considered as hits. A library of about 216 biologically active compounds were screened by distributing in four 96-well plates as single compounds at 10 mM, 1 mM, and 0.1 mM concentrations. The library was screened at 20 μM, 2 μM, and 0.2 μM final concentrations (FIG. 22). Three compounds were identified as primary hits: indolactam V (a PKC activator), γ-secretase inhibitor XX (a Notch inhibitor), and LDN193189 (a BMP inhibitor) (FIG. 2B and FIG. 22). An optimum concentration for each compound was determined (FIG. 2C and FIG. 24A) and used along with Alk5 inhibitor II. Addition of Indolactam V and 7-secretase inhibitor XX along with Alk5 inhibitor during stage 5 of differentiation not only increased the percentage of pre-delta cells though not significant, but also significantly reduced the polyhormonal cells population (FIG. 2H-I, FIGS. 24C, and FIG. 25A,C). However, omission of Alk5inh II resulted in significant reduction in monohormonal SC-delta cells (FIG. 24C). An increase in pre-delta cells from 4.11±1.12 percent to 8.76±0.16 percent was noted, as well as a significant reduction in polyhormonal cells from 54.88±6.74 percent to 30.59±1.10 percent (FIG. 25A-C), especially, SST⁺GCG⁺INS⁺ cells from 15.46±15.07 percent to 6.15±0.45 percent, and SST⁻GCG⁺INS⁺ cells from 32.97±1.71 percent to 16.30±1.29 percent (FIG. 25D). Also, there was significant decrease in SC-α cells population from 14.78±1.72 percent to 3.01±0.44 percent, and a significant increase in SC-β cells population from 5.20±0.72 percent to 18.0±1.47 percent (FIG. 25D). A significant increase in SST⁻GCG⁻INS⁻ cells from 20.97±7.59 percent to 39.54±2.51 percent with the use of Indolactam V and γ-secretase inhibitor XX along with Alk5i might indicate that these compounds block the generation of polyhormonal cells (FIG. 25D). Additionally, stage 5 differentiation has resulted in generation of 23.08±0.6 percent of total somatostatin positive cells, 48.01±1.99 percent of total insulin positive cells, and 26.15±1.62 percent of total glucagon positive cells (FIGS. 21, 3A, and 5B; and FIGS. 8A, 10B, and 15). The pre-delta cells express Pdx1, Pax6, Hhex, and Ptch1 (FIGS. 5A and 6A; and FIG. 10A). Majority of these cell population are negative for alpha cell marker Arx (FIG. 11A-B). Also, somatostatin protein was detected in western blot at this stage (FIG. 6D and FIG. 12A-B).

### Example 6: Maturation of SC-Delta Cells

When Alk5 inhibitor II, Indolactam V, and γ-secretase inhibitor XX were withdrawn from S3 media, no further increase in pre-delta cells was observed (8.76±0.16 v/s 8.15±10.85) (FIGS. 25A,F and 26B). Nevertheless, an increase in SC-β cells along with SC-8 cells population was observed during stage 4 and 5 of differentiation (FIG. 2I). Thus, to further increase the percentage of monohormonal SC-δ cells, a small molecule screen on pre-delta cells was conducted, as described earlier with a similar set of criteria (FIG. 2D). The compounds forskolin and indolactam V were identified as primary hits (FIG. 2E and FIG. 23). Both forskolin and Indolactam V showed dose dependent increase in monohormonal SCδ cells (FIG. 2F and FIG. 24B), however, using higher concentration led to severe cell loss. Although, use of Indolactam V along with forskolin significantly increased monohormonal SCδ cells (FIG. 24D), it aggravated cell loss. Therefore, pre-delta cells were treated only with 200 μM of forskolin for 7 days, an increase in SCδ cells from 8.15±0.85 percent to 17.51±0.88 percent was observed (FIG. 25E-G). Treatment with forskolin also resulted in 58.27±1.71 percent of total somatostatin positive cells, 47.28±1.94 percent of total insulin positive cells, and 19.92±2.29 percent of total glucagon positive cells (FIGS. 21, 3B, and 5B; and FIGS. 8A, 10B, and 16). Treatment with forskolin resulted in significant reduction in SC-α cells from 9.70±1.56 percent to 0.79±0.14 percent (FIG. 25H). Though there was no significant reduction in total polyhormonal cells, there was a significant reduction in SST⁺GCG⁺INS⁺ cells from 18.21±4.78 percent to 11.02±0.60 percent and SST⁻GCG⁺INS⁺ cells from 14.59±2.10 percent to 2.36±0.36 percent; meanwhile there was significant increase in SST⁺GCG⁻INS⁺ cells from 2.97±0.44 percent to 26.76±1.33 percent (FIG. 25H). The mRNA levels of somatostatin were significantly higher, while glucagon and insulin were significantly lower in SCδ cells compared to human cadaveric islets (FIG. 6B). Increased somatostatin protein from SCδ cells was also evident from western blots (FIG. 6C; and FIG. 12A-B). To further explore the stability of differentiated S6SCδ cells, forskolin was withdrawn from S3 media and cultured for additional two weeks. Surprisingly, a further increase in the percentage of SC-δ cells from 17.51±0.88 percent to 31.15±1.30 percent was observed, demonstrating that stem cell derived delta cells are stable under in vitro culture conditions (FIGS. 17 and 19C). The majority of S6SCδ cells were negative for Pdx1 and PC1 and were positive for Pax6, Hhex, Ptch1, CgA, CgB, and PC2 (FIGS. 5A and 6B; and FIG. 10A). The mRNA levels of Pdx1, Pax6, and hHEX in SCδ cells were significantly lower than that in human cadaveric islets & pre-delta cells (FIG. 6B). The mRNA levels of PTCH1 are comparable with that of human cadaveric islet (FIG. 6B). A significant higher mRNA level of alpha cell marker ARX in SCδ cells was present (FIG. 6B). As described earlier, forskolin not only reduced the expression of glucagon but also alpha cell marker Arx (FIG. 11A-B).

### Example 7: NKX6.1 Expression in Pancreatic Progenitors Reduces Differentiation to SC-δ Cells

Following a SC-β protocol (see, e.g., Pagliuca et al., Cell, 159:428-439 (2014)), Nkx6.1 expression was induced in pancreatic progenitors before differentiating them into delta progenitor cells (FIG. 4A). 24.14±1.18 percent of PP cells were co-positive for PDX1 and NKX6.1 (FIG. 4B and FIG. 13B). Induction of NKX6.1 in PP cells significantly reduced the percentage of monohormonal SC-δ cells formed during stage 6 of differentiation, though there was no significant difference noted during stage 4 and stage 5 (FIG. 4C,F and FIG. 9D). This could be due to significantly reduced endocrine differentiation at stage 5 and 6 (FIG. 4D,F and FIG. 9D) and increased number of cells expressing NKX6.1, as evident by immunocytochemical staining of S6SCδ cells (FIG. 4E and FIG. 9C). A significantly reduced mRNA levels in delta progenitor, pre-delta, and SCδ cells was noted upon differentiation of PP cells following SC-δ protocol (FIG. 6B). Furthermore, reduced SC-δ cells differentiation was supported by reduced somatostatin protein observed in western blot analysis (FIG. 6D and FIG. 12C-D). Though reduction in SC-δ cells differentiation following SC-β protocol was noted, the differentiated SC-δ cells expressed pancreatic delta cells specific markers Hhex and Ptch1 (FIG. 9A-B).

### Example 8: Characterization of Subpopulation During Differentiation of SC-δ Cells

Experiments were conducted to identify different population of cells that are present during differentiation by using flow cytometry. The cells were stained with antibodies for glucagon, insulin, and somatostatin. The quadrant plots were drawn for glucagon versus somatostatin, insulin versus somatostatin, and glucagon versus insulin (FIG. 18B). Using gating strategies as shown in FIG. 18C, the following population of cells were identified: three monohormonal population of cells, namely SST⁻GCG⁻INS⁻ (SC-α), SST⁻GCG⁻INS⁺ (SC-β), and SST⁺GCG⁻INS⁻ (SC-δ); four polyhormonal population of cells, namely SST⁺GCG⁻INS⁺, SST⁺GCG⁺INS⁻, SST⁻GCG⁺INS⁺, SST⁺GCG⁺INS*; and one non-hormonal population of cells namely SST⁻GCG⁻INS⁻. A gradual increase in SC-δ cells population was observed with progress in differentiation, the population of SC-α and SC-β was higher during stage 5 of differentiation (FIG. 19A), also a similar trend was observed in these populations following SC-β protocol though with lesser number (FIG. 19B). The use of Indolactam V and γ-secretase inhibitor XX during stage 5 significantly reduced SC-α cells population from 14.78±1.72 percent to 3.01±0.44 percent, and significantly increased SC-β cells population from 5.20±0.72 percent to 18.01±1.47 percent (FIG. 25D). For efficient SC-δ differentiation, the SC-β protocol was modified, as there was no change in the number of non-hormonal cells during stage 4, 5, and 6 of differentiation (FIG. 19B). There was a gradual increase in polyhormonal cells population (namely SST⁺GCG⁻INS⁺, SST⁺GCG⁺INS⁻, and SST⁺GCG⁺INS⁺) with progress in differentiation. However, the polyhormonal SST⁻GCG⁺INS⁺ population was higher during stage 5 of differentiation (FIG. 19A). With prolonged culture without any factors, results surprisingly showed further increase in the population of SC-δ cells and a decrease in SC-β cells (FIG. 19C). However, there was an increase in SC-α cell population indicating forskolin might have prevented SC-α cells differentiation during stage 6 since treatment of forskolin significantly reduced the number of SC-α cells from 9.70±1.56 percent to 0.79±0.14 percent (FIGS. 19C and 25H).

### Example 9: Functional Characterization of SC-8 Cells

Differentiating SC-δ cell clusters were sequentially challenged with 3.3 mM and 16.5 mM of glucose. There was noted secretion of somatostatin in response to glucose challenge. Higher somatostatin secretion was observed under low glucose condition (FIGS. 1F and 3C-D). Delta progenitor cells secreted significantly less somatostatin compare to human cadaveric islets, however, both pre-delta and SCδ cells were capable of secreting significantly higher amount of somatostatin than human islets (FIGS. 1F and 3C-D). Since generation of SC-β, SC-α, and polyhormonal cells in addition to SC-δ cells was noted during differentiation, experiments were conducted to measure the levels of insulin and glucagon secreted upon glucose challenge. Measured levels of insulin and glucagon in SC-δ cells were significantly lower than that observed in SC-β and SC-α cells. The secretion pattern of insulin resembled immature and/or dysfunctional beta cells, and more glucagon secretion was observed under low glucose condition except at stage 5 of differentiation (FIGS. 1G-H and 4E-H).

Pancreatic delta cells can inhibit the activity of both alpha and beta cells through paracrine signaling by secreting somatostatin. Experiments were conducted to investigate the effect of conditioned medium (CM), collected on 6^{th} day of stage 6 from differentiating S6SCδ cells, on glucose responsiveness of SC-β and SC-α cells. Both SC-β and SC-α cells cultured overnight with CM were subjected to glucose challenge, and the level of secreted insulin and glucagon was assessed. SC-δ cells CM inhibited secretion of glucagon from SC-α cells but not insulin from SC-β cells (FIG. 3I-J).

The formation of packaged somatostatin granules was confirmed by transmission electron microscopy (FIG. 5C and FIG. 11C). The average size of secretory granules in delta progenitor cells is 187±0.05 nm, in S5SCδ cells is 223±0.01 nm, and in S6SCδ cells is 179±0.004 nm (FIG. 11D). These results are in accordance with an average size of human somatostatin granule (e.g., about 250 nm). Also, these granules are spherical and non-lozenge shaped. The delta progenitor cells granules are moderately opaque, which gradually becomes denser during stage 5 and stage 6 of differentiation.

### Example 10: Further Applications

The precise control of glycaemia can include proper functioning pancreatic endocrine cells in the islet of Langerhans, and the function of these cells can rely on intra-islet autocrine and paracrine signaling. Disturbance to this complex signaling network can lead to loss of control of glycaemia. Destruction of beta cells by patient's own immune system can lead to Type 1 diabetes. Whereas, either dysfunction in endocrine cells or resistance to their secreted hormones by themselves or peripheral tissues can result in Type 2 diabetes. While islet transplantation has proven to be successful for treatment of Type 1 diabetes, due to lack of cadaveric islets availability, recently, cell therapy has gained much attention. With the application of directed differentiation, it may now be possible to convert pluripotent stem cells (e.g., ESCs and/or iPSCs) into pancreatic endocrine cells (see, e.g., as described herein and as described in Pagliuca et al., Cell, 159:428-439 (2014); Peterson et al., Nat. Commun., 11:Article No. 2241 (14 pages) (2020); Rezania et al., Nat. Biotechnol., 32:1121-1133 (2014); and Russ et al., EMBO J., 34:1759-1772 (2015); and Rezania et al., Diabetes, 60:239-247 (2011)). Such cells can then be ectopically transplanted, unlike most other stem cell derived cells that may involve their integration into diseased tissues for proper functioning (see, e.g., Merani et al., British J. Surgery, 95:1449-1461 (2008)).

Although prior work reported successful generation of SC-α and SC-β cells using pluripotent stem cells, differentiation of other endocrine cell types was not effective possibly partly due to the lack of understanding complex developmental signals, especially the temporal and spatial cues that govern pancreatic tissue development. Described herein is a small molecule PD173074, which along with retinoic acid (RA), was capable of differentiating human ESCs derived pancreatic progenitors (PP) into delta progenitor (DP) cells, which can to a degree further be differentiated into SC-δ cells spontaneously. Given the complexity of endocrine differentiation and the paucity of information known about specification of delta cells, described herein is a generalized approach (e.g., a combinatorial screen) to screen for combinations of signals involved in the differentiation of PP cells to somatostatin expressing delta progenitor cells. In some cases, a combination of signals can be used to designate cell fate during differentiation rather than a single signal. Indeed, directed differentiation protocols can use sequential induction of different intermediate cells with different combinations of cocktails of growth factors and small molecules to get final cell products. Therefore, experiments herein can be used to implement a Pathway and Interaction Elucidation through Combinatorial Chemical Screening (PIECCS) approach capable of evaluating the interactions of several small molecules in concert to identify signaling combinations.

In conventional small molecule screening, false positives and false negatives can complicate the interpretation of results and lead to failed compounds. The PIECCS screening approach herein can be used to mitigate these concerns due to the nature of the combinatorial library. Each individual compound is included in the screening library hundreds of times. Data analysis incorporates all these replicates into a single statistical model significantly reducing the influence of false positive/negatives, positional effects, and thereby increasing statistical confidence in the results. The PIECCS approach used herein successfully identified pathways that were previously unknown to be involve in delta cell differentiation. This approach can easily be adapted to identify pathways that are involved in the differentiation of other cell types.

Using this combinatorial screening approach, the involvement of FGF and RA signaling in the directed differentiation of delta cells was revealed. Neither of these two pathways had previously been implicated in the differentiation of delta cells. The opposing effect of FGF and RA signaling has been demonstrated in other tissues including, e.g., limb development and somitogenesis. Herein, retinoic acid induced somatostatin expression in PP cells under the absence of FGF signaling. RA alone was unable to induce sufficient somatostatin expression in these PP cells (FIG. 1I), suggesting SC-δ cell specification unlike SC-u and SC-β cells may include blockage of FGF signaling under continuous active RA signal at this stage. The level of induction of somatostatin in PP cells was lower following stage 3 of the SC-α and SC-β protocol; and methods herein can include modifications to SC-α and SC-β protocols to enhance induction of somatostatin, thereby providing PP cells for further SC-δ cells derivation. Further, induction of NKX6.1 expression in PP cells was reduced, and its inhibition enhanced the endocrine differentiation. Although recently reported SC-α generation may use PDX1 positive and NKX6.1 negative PP cells, induction of alpha-lineage was efficient with timely activation of RA signaling under the absence of BMP but not FGF signal. In protocols for generating SC-δ cells, it is possible that the combination of PD173074 and RA may not only drive differentiation towards delta cells, but instead may block differentiation to other endocrine cell types. Given that the resulting SC-δ cells continue to express ARX, methods herein can be adapted to include one or more factors or conditions that can reduce or suppress the expression of this gene. Once delta progenitor cells are formed, further differentiation to a delta cell fate may include simultaneous activation of protein kinase C (PKC) and inhibition of TGF-β and Notch signaling, which in turn can increase both SC-δ and polyhormonal cell populations. TGF-β and Notch signaling have been implicated in many developmental processes, including pancreatic development. Blocking TGF-β signaling in early embryonic pancreas can cause differentiation of pancreatic epithelial cells into endocrine cells, while in adult mice it can increase beta cell mass through replication. Notch signaling can control pancreatic fate decision through lateral inhibition; and its activation in pancreatic progenitors can prevent their differentiation into endocrine or exocrine cell lineages, while its blockage can cause differentiation of progenitors into endocrine cells. Thus, Notch signaling may be used to maintain a pool of progenitor cells as well as prevent them from undergoing precocious endocrine differentiation. Without wishing to be limited by any particular mechanism, simultaneous inhibition of both TGF-β and Notch signaling in delta progenitor cells might have blocked glucagon expression, while activation of PKC signaling maintained PDX1 expression in these cells. However, inhibition of TGF-β alone may not be sufficient to block the expression of glucagon resulting in the generation of more polyhormonal cells (FIG. 25B-D).

Further, treatment of stage 5 SC-δ cells with forskolin increased monohormonal SC-5 cells while reducing glucagon expression. This was further evident from long term culture, in which withdrawal of forskolin from media resulted in an increase in SC-α cells population (FIG. 19C). Forskolin can be an activator of adenylyl cyclase and can increase the concentration of cyclic AMP (cAMP), thereby eliciting cAMP-dependent physiological responses. Rat somatostatin gene harbors cAMP response element (CRE) upstream of its transcriptional initiation site and is involved in tissue-specific somatostatin gene expression, mutation of which results in a significant loss of transcriptional activity. Without wishing to be limited by any particular mechanism, forskolin might be involved in cAMP-dependent increase in somatostatin gene expression in stage 5 completed SC-δ cells. Further studies may include, e.g., exploring the reason for reduction in SC-α and SC-β cells after forskolin treatment. Furthermore, in some instances, SC-δ cells can be terminally differentiated and can be neither proliferative nor undergoing apoptosis during stage 4, 5, and 6 of differentiation (FIGS. 20A-B and 21A-B).

When delta progenitor cells formed following stage 3 of either SC-α or SC-β protocol are further differentiated, such protocols were unable to generate sufficient SC-δ cells. These observations highlight the use of specific signals for different endocrine subtype cells specification.

1. A method for producing a cell population comprising delta progenitor (DP) cells, wherein said method comprises: culturing a first population of pancreatic progenitor (PP) cells in the presence of a fibroblast growth factor receptor inhibitor and a retinoic acid signaling pathway activator, wherein said cell population comprises delta progenitor cells produced from said PP cells of said first population.

2. The method of claim 1, wherein said PP cells are PDX1⁺/NKX6.1⁻ cells.

3. The method of claim 2, wherein said first population of PP cells comprises more than about 80%, 85%, 90%, or 95% Pdx1 positive cells.

4. The method of claim 2, wherein said first population of PP cells comprises less than about 10% Nkx6.1 positive cells.

5-9. (canceled)

10. The method of claim 1, wherein said cell population of delta progenitor cells comprises a second population comprising a plurality of somatostatin-positive cells or precursors thereof.

11. The method of claim 10, wherein said cell population of delta progenitor cells comprises a third population of a plurality of glucagon-positive cells, and where said second population of said cell population is greater than said third population of said cell population.

12. A cell population comprising:
(a) delta progenitor (DP) cells, wherein said delta progenitor cells of said cell population are derived from a first population comprising pancreatic progenitor (PP) cells cultured in the presence of a fibroblast growth factor receptor inhibitor and a retinoic acid signaling pathway activator, and wherein said delta progenitor cells of said cell population express somatostatin, glucagon, insulin, or a combination thereof;
(b) delta progenitor (DP) cells, wherein said delta progenitor cells of said cell population are produced using a method comprising culturing a first population of pancreatic progenitor (PP) cells in the presence of a fibroblast growth factor receptor inhibitor and a retinoic acid signaling pathway activator, and wherein said delta progenitor cells of said cell population express somatostatin, glucagon, insulin, or a combination thereof;
(c) pre-delta (PD) cells, wherein said pre-delta cells of said cell population are produced using a method comprising (i) culturing a first population of pancreatic progenitor (PP) cells in the presence of a fibroblast growth factor receptor inhibitor and a retinoic acid signaling pathway activator to form a second population of delta progenitor (DP) cells, and (ii) culturing said second population of delta progenitor cells in the presence of a transforming growth factor-β (TGF-β) signaling inhibitor to form said cell population of pre-delta cells, and wherein said pre-delta cells of said cell population express somatostatin, glucagon, insulin, or a combination thereof;
(d) stem cell-derived delta (SC-delta) cells, wherein said SC-delta cells of said cell population are derived from a first population comprising pre-delta (PD) cells cultured in the presence of an adenylyl cyclase activator, and wherein said SC-delta cells of said cell population express somatostatin, glucagon, insulin, or a combination thereof;
(e) stem cell-derived delta (SC-delta) cells, wherein said SC-delta cells of said cell population are produced using a method comprising culturing a first population of pre-delta (PD) cells in the presence of an adenylyl cyclase activator, and wherein said SC-delta cells of said cell population express somatostatin, glucagon, insulin, or a combination thereof; or
(f) stem cell-derived delta (SC-delta) cells, wherein said SC-delta cells of said cell population are produced using a method comprising (i) culturing a first population of stem cells in the presence of a growth factor from a transforming growth factor-β (TGF-β) superfamily and a glycogen synthase kinase 3 (GSK3) inhibitor to form a second population of definitive endoderm (DE) cells, (ii) culturing said second population of DE cells in the presence of a first growth factor from a fibroblast growth factor (FGF) family to form a third population of gut tube endoderm (GTE) cells, (iii) culturing said third population of GTE cells in the presence of a second growth factor from a fibroblast growth factor (FGF) family, a first protein kinase C (PKC) activator, a bone morphogenic protein (BMP) signaling pathway inhibitor, a sonic hedgehog (SHH) pathway antagonist, and a first retinoic acid (RA) signaling pathway activator to form a fourth population of pancreatic progenitor (PP) cells, (iv) culturing said fourth population of PP cells in the presence of a fibroblast growth factor receptor (FGFR) inhibitor and a second RA signaling pathway activator to form a fifth population of delta progenitor (DP) cells, (v) culturing said fifth population of delta progenitor cells in the presence of a TGF-β signaling inhibitor, or a combination of a second PKC activator and a Notch signaling inhibitor, or a combination of a TGF-β signaling inhibitor, a second PKC activator, and a Notch signaling inhibitor to form a sixth population of pre-delta (PD) cells, and (vi) culturing said sixth population of pre-delta cells in the presence of an adenylyl cyclase activator to form said cell population of SC-delta cells, and wherein said SC-delta cells of said cell population express somatostatin, glucagon, insulin, or a combination thereof.

13. The cell population of claim 12, wherein at least three percent of said cell population of (a) or (b) are delta progenitor cells.

14. The cell population of claim 12, wherein at least five percent or 15 percent of said cell population of (a) or (b) are delta progenitor cells that express somatostatin.

15. The cell population of claim 12, wherein five percent or less of said cell population of (a) or (b) are delta progenitor cells that express glucagon.

16. The cell population of claim 12, wherein 15 percent or less of said cell population of (a) or (b) are delta progenitor cells that express insulin.

17-28. (canceled)

29. A method for producing a cell population comprising pre-delta (PD) cells, wherein said method comprises:
(a) culturing a first population of pancreatic progenitor (PP) cells in the presence of a fibroblast growth factor receptor inhibitor and a retinoic acid signaling pathway activator to form a second population of delta progenitor (DP) cells, and
(b) culturing said second population of delta progenitor cells in the presence of a transforming growth factor-β (TGF-β) signaling inhibitor to form said cell population of pre-delta cells.

30. The method of claim 29, wherein said PP cells are PDX1⁺/NKX6.1⁻ cells.

31. The method of claim 30, wherein said first population of PP cells comprises more than about 80% Pdx1 positive cells.

32. The method of claim 30, wherein said first population of PP cells comprises less than about 10% of Nkx6.1 positive cells.

33-37. (canceled)

38. The method of claim 29, wherein said second population of delta progenitor cells comprises a third population comprising a plurality of somatostatin-positive cells or precursors thereof.

39. The method of claim 38, wherein said second population of delta progenitor cells comprises a fourth population of a plurality of glucagon-positive cells or a fifth population of a plurality of insulin-positive cells or a combination of said fourth population and said fifth population, and optionally wherein said third population is greater than said fourth population.

40-42. (canceled)

43. The method of claim 29, wherein 5 percent or less of said cell population of pre-delta cells are somatostatin-positive cells.

44. The method of claim 29, wherein said cell population of pre-delta cells comprises a population SC-delta cells, SC-alpha cells, SC-beta cells, non-hormonal cells, polyhormonal cells, or a combination thereof.

45-52. (canceled)

53. A method for producing a cell population comprising stem cell-derived delta (SC-delta) cells, wherein:
(a) said method comprises culturing a first population of pre-delta (PD) cells in the presence of an adenylyl cyclase activator, wherein said cell population comprises SC-delta cells produced from said pre-delta cells of said first population;
(b) said method comprises:
(i) culturing a first population of delta progenitor (DP) cells in the presence of a protein kinase C activator and a Notch signaling inhibitor to form a second population of pre-delta (PD) cells, and
(ii) culturing said second population of pre-delta cells in the presence of an adenylyl cyclase activator to form said cell population of SC-delta cells:
(c) said method comprises:
(i) culturing a first population of stem cells in the presence of a growth factor from a transforming growth factor-β (TGF-β) superfamily and a glycogen synthase kinase 3 (GSK3) inhibitor to form a second population of definitive endoderm (DE) cells,
(ii) culturing said second population of DE cells in the presence of a first growth factor from a fibroblast growth factor (FGF) family to form a third population of gut tube endoderm (GTE) cells,
(iii) culturing said third population of GTE cells in the presence of a second growth factor from a fibroblast growth factor (FGF) family, a first protein kinase C (PKC) activator, a bone morphogenic protein (BMP) signaling pathway inhibitor, a sonic hedgehog (SHH) pathway antagonist, and a first retinoic acid (RA) signaling pathway activator to form a fourth population of pancreatic progenitor (PP) cells,
(iv) culturing said fourth population of PP cells in the presence of a fibroblast growth factor receptor (FGFR) inhibitor and a second RA signaling pathway activator to form a fifth population of delta progenitor (DP) cells,
(v) culturing said fifth population of delta progenitor cells in the presence of a TGF-β signaling inhibitor, or a combination of a second PKC activator and a Notch signaling inhibitor, or a combination of a TGF-β signaling inhibitor, a second PKC activator, and a Notch signaling inhibitor to form a sixth population of pre-delta (PD) cells, and
(vi) culturing said sixth population of pre-delta cells in the presence of an adenylyl cyclase activator to form said cell population of SC-delta cells: or
(d) said method comprises:
(i) culturing a first population of pancreatic progenitor (PP) cells in the presence of a fibroblast growth factor receptor (FGFR) inhibitor and a retinoic acid (RA) signaling pathway activator to form a second population of delta progenitor (DP) cells,
(ii) culturing said second population of delta progenitor cells in the presence of a transforming growth factor-β (TGF-β) signaling inhibitor, or a combination of a protein kinase C (PKC) activator and a Notch signaling inhibitor, or a combination of a TGF-β signaling inhibitor, a PKC activator, and a Notch signaling inhibitor to form a third population of pre-delta (PD) cells, and
(iii) culturing said third population of pre-delta cells in the presence of an adenylyl cyclase activator to form said cell population of SC-delta cells.

54-131. (canceled)


SEQUENCE LISTING (26 sequences)

SEQ ID NO: 1 - DNA, 20 bp, synthetic construct
  source 1..20: mol_type = other DNA
cgtccagctgcctttcccat

SEQ ID NO: 2 - DNA, 25 bp, synthetic construct
  source 1..25: mol_type = other DNA
ccgtgagatgtacttgttgaaagga

SEQ ID NO: 3 - DNA, 20 bp, synthetic construct
  source 1..20: mol_type = other DNA
ccgagtcctgcttcttcttg

SEQ ID NO: 4 - DNA, 20 bp, synthetic construct
  source 1..20: mol_type = other DNA
attcgttggggatgacagag

SEQ ID NO: 5 - DNA, 24 bp, synthetic construct
  source 1..24: mol_type = other DNA
aaccaattccacaacccaccacac

SEQ ID NO: 6 - DNA, 24 bp, synthetic construct
  source 1..24: mol_type = other DNA
ttatttgccatggtgaagctgggc

SEQ ID NO: 7 - DNA, 32 bp, synthetic construct
  source 1..32: mol_type = other DNA
catgttcagaaaactggatttaggaataatgt

SEQ ID NO: 8 - DNA, 33 bp, synthetic construct
  source 1..33: mol_type = other DNA
cctaagagcagtacataaactatttgttaagtc

SEQ ID NO: 9 - DNA, 20 bp, synthetic construct
  source 1..20: mol_type = other DNA
gttgtgggcctcctcatatt

SEQ ID NO: 10 - DNA, 21 bp, synthetic construct
  source 1..21: mol_type = other DNA
gacttactcgtcctccaattc

SEQ ID NO: 11 - DNA, 22 bp, synthetic construct
  source 1..22: mol_type = other DNA
ctgctgaaacgcaaacagaggc

SEQ ID NO: 12 - DNA, 22 bp, synthetic construct
  source 1..22: mol_type = other DNA
ctcggtcaagtccagcctcatg

SEQ ID NO: 13 - DNA, 20 bp, synthetic construct
  source 1..20: mol_type = other DNA
ggcatcacctacatgctctt

SEQ ID NO: 14 - DNA, 20 bp, synthetic construct
  source 1..20: mol_type = other DNA
aagaaaccctggaacgactg

SEQ ID NO: 15 - DNA, 20 bp, synthetic construct
  source 1..20: mol_type = other DNA
gcgatggcagatcagaaaga

SEQ ID NO: 16 - DNA, 21 bp, synthetic construct
  source 1..21: mol_type = other DNA
agagctgaagactgagagcta

SEQ ID NO: 17 - DNA, 20 bp, synthetic construct
  source 1..20: mol_type = other DNA
ccaaccagacggagaatgat

SEQ ID NO: 18 - DNA, 20 bp, synthetic construct
  source 1..20: mol_type = other DNA
ccatagccgggtttgagtta

SEQ ID NO: 19 - DNA, 20 bp, synthetic construct
  source 1..20: mol_type = other DNA
cagcctttgtgaaccaacac

SEQ ID NO: 20 - DNA, 22 bp, synthetic construct
  source 1..22: mol_type = other DNA
ggtcttgggtgtgtagaagaag

SEQ ID NO: 21 - DNA, 19 bp, synthetic construct
  source 1..19: mol_type = other DNA
tgccaaacgtcacgatgaa

SEQ ID NO: 22 - DNA, 19 bp, synthetic construct
  source 1..19: mol_type = other DNA
tcaccagccaagcaatgaa

SEQ ID NO: 23 - DNA, 21 bp, synthetic construct
  source 1..21: mol_type = other DNA
atcaccatcttccaggagcga

SEQ ID NO: 24 - DNA, 21 bp, synthetic construct
  source 1..21: mol_type = other DNA
ttctccatggtggtgaagacg

SEQ ID NO: 25 - protein, 27 aa, unidentified
  MOD_RES 27: note = modification at Leu-27 to include a C-terminal amide
HSDGIFTDSYSRYRKQMAVKKYLAAVL

SEQ ID NO: 26 - protein, 38 aa, synthetic construct
  MOD_RES 38: note = modification at Lys-38 to have a C-terminal amide
HSDGIFTDSYSRYRKQMAVKKYLAAVLGKRYKQRVKNK